(19) United States
(12) Patent Application Publication
(10) Pub. No.: US 2026/0152736 A1
(43) Pub. Date: Jun. 4, 2026

(54) COMPOSITIONS AND METHODS FOR PURIFYING POLYRIBONUCLEOTIDES

(71) Applicant: Flagship Pioneering Innovations VI. LLC, Cambridge, MA (US)

(72) Inventors: Alexandra Sophie DE BOER, Somerville, MA (US); Ki Young PAEK, Brighton, MA (US)

(21) Appl. No.: 19/125,395

(22) PCT Filed: Oct. 30, 2023
(86) PCT No.: PCT/US2023/078207

§ 371 Date: Apr. 29, 2025

Related U.S. Application Data
(60) Provisional application No. 63/420,984, filed on Oct. 31, 2022.

(51) Int. Cl. C12N 15/10; A61K 31/7088; C12N 15/115

(52) U.S. Cl. CPC C12N 15/10; A61K 31/7088; C12N 15/115; C12N 2310/16; C12N 2310/532; C12N 2330/00; C12N 2840/203

(57) ABSTRACT

The present disclosure relates to compositions and methods for separating and/or purifying polyribonucleotides. The polyribonucleotide may be separated from a mixture of polyribonucleotides with a reagent that binds an aptamer on the polyribonucleotide.

### SEQUENCE LISTING

This application contains a Sequence Listing which has been filed electronically in Extensible Markup Language (XML) format and is hereby incorporated by reference in its entirety. Said XML copy, created on Oct. 30, 2023, is named 51509-067WO2_Sequence_Listing_10_27_23.XML and is 143,826 bytes in size.

### BACKGROUND

Polyribonucleotides are useful for a variety of therapeutic and engineering applications. Thus, new compositions and methods for separating and purifying polyribonucleotides are needed.

### SUMMARY OF THE INVENTION

In one aspect, the disclosure features a method of separating a linear polyribonucleotide having an aptamer from a plurality of polyribonucleotides that includes a mixture of linear polyribonucleotides and circular polyribonucleotides. The method includes the steps of (a) providing a sample that includes the plurality of polyribonucleotides, wherein a subset of the plurality of polyribonucleotides include the linear polyribonucleotide having the aptamer; (b) contacting the sample with a reagent that binds to the aptamer; and (c) separating the linear polyribonucleotide having the aptamer that is bound to the reagent from the plurality of polyribonucleotides.

In some embodiments, the linear polyribonucleotide with the aptamer is transcribed from a deoxyribonucleotide encoding the linear polyribonucleotide including the aptamer.

In some embodiments, the method further includes producing the linear polyribonucleotide with the aptamer by attaching the aptamer to the linear polyribonucleotide.

In another aspect, the disclosure features a method of separating a linear polyribonucleotide from a plurality of polyribonucleotides that includes linear polyribonucleotides and circular polyribonucleotides.

The method includes the steps of (a) providing a sample that includes the plurality of polyribonucleotides, wherein a subset of the plurality of polyribonucleotides include the linear polyribonucleotide; (b) attaching an aptamer to the linear polyribonucleotide; and (c) contacting the sample with a reagent that binds to the aptamer; and (d) separating the linear polyribonucleotide having the aptamer that is bound to the reagent from the plurality of polyribonucleotides.

In some embodiments, the step of attaching the aptamer to the linear polyribonucleotide includes covalently attaching the aptamer to a 3′ or 5′ terminus of the linear polyribonucleotide.

In some embodiments, the step of attaching the aptamer to the linear polyribonucleotide includes hybridizing the aptamer to a region of the linear polyribonucleotide.

In some embodiments of either of the foregoing aspects, the circular polyribonucleotides lack the aptamer.

In some embodiments, the step of separating includes collecting a portion of the sample that is not bound by the reagent. For example, the portion of the sample that is not bound by the reagent may include the circular polyribonucleotide.

The reagent may be, for example, a polypeptide, a small molecule, a lipid, a carbohydrate, an RNA, or a metal.

In some embodiments, the reagent is a polypeptide. The polypeptide may be, for example, Protein A, streptavidin, lambda peptide, or MS2 bacteriophage coat protein. The polypeptide may be selected from Table 1.

In some embodiments, the reagent is a small molecule. The small molecule may be, for example, biotin or tetracycline. In some embodiments, the small molecule is a metabolite or an amino acid. In some embodiments, the small molecule is selected from Table 2.

In some embodiments, the reagent is a carbohydrate.

In some embodiments, the aptamer includes a nucleic acid sequence selected from any one of SEQ ID NOs: 1-124.

In some embodiments, the aptamer may include a nucleic acid sequence having at least 85% (e.g., at least 90%, 95%, 97%, 99%, or 100%) sequence identity to an aptamer as shown in Table 1. The reagent may be a corresponding reagent as shown in Table 1 (e.g., any one of SEQ ID NOs: 1-66).

In some embodiments, the aptamer may include a nucleic acid sequence having at least 85% (e.g., at least 90%, 95%, 97%, 99%, or 100%) sequence identity to an aptamer as shown in Table 2 (e.g., any one of SEQ ID NOs: 67-119). The reagent may be a corresponding reagent as shown in Table 2.

In some embodiments, the aptamer may include a nucleic acid sequence having at least 85% (e.g., at least 90%, 95%, 97%, 99%, or 100%) sequence identity to an aptamer as shown in Table 3. The reagent may be a corresponding reagent as shown in Table 3 (e.g., SEQ ID NOs: 120 or 121).

In some embodiments, the aptamer may include a nucleic acid sequence having at least 85% (e.g., at least 90%, 95%, 97%, 99%, or 100%) sequence identity to an aptamer as shown in Table 4. The reagent may be a corresponding reagent as shown in Table 4 (e.g., any one of SEQ ID NOs: 122-124).

In some embodiments, the step of separating includes immobilizing the reagent.

In some embodiments, the reagent is conjugated to a particle. The particle may include, for example, a magnetic bead. In some embodiments, the reagent is conjugated to a resin that includes a plurality of the particles. The resin may include, for example, cross-linked poly[styrene-divinylbenzene], agarose, or SEPHAROSE®.

In some embodiments, a column includes the resin. The method may include contacting the sample with the column and collecting an eluate that includes a portion of the sample that is not bound to the reagent from the plurality of polyribonucleotides in the sample.

In some embodiments, the method further includes, prior to step (a), providing a linear precursor polyribonucleotide and circularizing the linear precursor polyribonucleotide to produce the circular polyribonucleotide. The linear precursor may include a 5′ self-splicing intron fragment and a 3′ self-splicing intron fragment. The circular polyribonucleotide may be produced by self-splicing of the linear precursor. The 5′ self-splicing intron fragment and the 3′ self-splicing intron fragment may each be a Group I or Group II self-splicing intron fragment. In some embodiments, circularizing the circular polyribonucleotide is produced by splint-ligation of the linear precursor.

In some embodiments, the circular polyribonucleotide includes an open reading frame (ORF). The ORF may encode a polypeptide. In some embodiments, a level of expression from the ORF of the circular polyribonucleotide after purification is increased at least 10% (e.g., at least 20%, 30%, 40%, 50%, 60%, 70%, 80%, 90%, 100%, or more) relative to a level of expression from the ORF prior to separating.

In some embodiments, the circular polyribonucleotide includes at least one internal ribosome entry site (IRES) (e.g., an IRES). The ORF may be operably linked to the IRES.

In some embodiments, the step of separating further includes washing the polyribonucleotide having the aptamer that is bound to the reagent one or more times.

In some embodiments, the step of separating further includes eluting the polyribonucleotide having the aptamer from the reagent.

In some embodiments, the method includes providing a plurality of reagents, wherein each reagent binds to a distinct aptamer region.

In some embodiments, the method includes providing the reagent at a molar ratio of 10:1 to 1:10 (e.g., 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10) to the polyribonucleotide including the aptamer region.

In some embodiments, the method separates at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, or more) of the circular polyribonucleotide.

In some embodiments, the method separates from 500 μg to 1000 mg (e.g., 500 μg to about 1 mg, e.g., about 600 μg, 700 μg, 800 μg, 900 μg, or 1 mg, e.g., from about 1 mg to about 10 mg, e.g., about 2 mg, 3 mg, 4 mg, 5 mg, 6 mg, 7 mg, 8 mg, 9 mg, or 10 mg, e.g., from about 10 mg to about 100 mg, e.g., about 20 mg, 30 mg, 40 mg, 50 mg, 60 mg, 70 mg, 80 mg, 90 mg, or 100 mg, e.g., from about 100 mg to about 1,000 mg, e.g., 200 mg, 300 mg, 400 mg, 500 mg, 600 mg, 700 mg, 800 mg, 900 mg, or 1000 mg) of the circular polyribonucleotide.

In another aspect, the disclosure features a population of polyribonucleotides produced by a method as described herein. The population may include a circular polyribonucleotide lacking the aptamer, and the circular polyribonucleotide includes at least 40% (e.g., at least 50%, 60%, 70%, 80%, 905, 95%, 97%, 99%, or more) (mol/mol) of the total polyribonucleotides in the composition. In some embodiments, the population includes less than 40% (e.g., less than 30%, 20%, 10%, 5%, or less) (mol/mol) linear polyribonucleotides of the total polyribonucleotides in the composition.

In some embodiments, a total weight of polyribonucleotides in the population of polyribonucleotides at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1000 mg, or more).

In some embodiments, the total weight of polyribonucleotides in the population of polyribonucleotides is from 500 μg to 1000 mg (e.g., 500 μg to about 1 mg, e.g., about 600 μg, 700 μg, 800 μg, 900 μg, or 1 mg, e.g., from about 1 mg to about 10 mg, e.g., about 2 mg, 3 mg, 4 mg, 5 mg, 6 mg, 7 mg, 8 mg, 9 mg, or 10 mg, e.g., from about 10 mg to about 100 mg, e.g., about 20 mg, 30 mg, 40 mg, 50 mg, 60 mg, 70 mg, 80 mg, 90 mg, or 100 mg, e.g., from about 100 mg to about 1,000 mg, e.g., 200 mg, 300 mg, 400 mg, 500 mg, 600 mg, 700 mg, 800 mg, 900 mg, or 1000 mg).

In another aspect, the disclosure features a composition that includes a population of polyribonucleotides as described herein (e.g., produced by a method as described herein) and a diluent, carrier, or excipient.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity but include the general class of which a specific example may be used for illustration. The term “or” is used to mean “and/or” unless explicitly indicated to refer to alternatives only or the alternative are mutually exclusive, although the disclosure supports a definition that refers to only alternatives and “and/or.” The terminology herein is used to describe specific embodiments, but their usage is not to be taken as limiting, except as outlined in the claims.

As used herein, any values provided in a range of values include both the upper and lower bounds, and any values contained within the upper and lower bounds.

As used herein, the term “about” refers to a value that is within ±10% of a recited value.

As used herein, the term “aptamer” is a polynucleotide that specifically binds to a molecule (e.g., a reagent). An aptamer may be a portion of a polyribonucleotide molecule. Typically, an aptamer is from 5 to 500 nucleotides (e.g., between 5 and 200, 5 and 150, 5 and 100, 5 and 50, 10 and 200, 10 and 150, 10 and 100, 10 and 50, 20 and 200, 20 and 150, 20 and 100, or 20 and 50 nucleotides). An aptamer binds to its target through secondary structure rather than sequence homology.

As used herein, the term “carrier” is a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the terms “circular polyribonucleotide,” “circular RNA,” and “circRNA” are used interchangeably and mean a polyribonucleotide molecule that has a structure having no free ends (i.e., no free 3′ or 5′ ends), for example a polyribonucleotide molecule that forms a circular or end-less structure through covalent or non-covalent bonds. The circular polyribonucleotide may be, e.g., a covalently closed polyribonucleotide.

As used herein, the terms “disease,” “disorder,” and “condition” each refer to a state of sub-optimal health, for example, a state that is or would typically be diagnosed or treated by a medical professional.

As used herein, the term “expression sequence” is a nucleic acid sequence that encodes a product, e.g., a peptide or polypeptide. An exemplary expression sequence that codes for a peptide or polypeptide can include a plurality of nucleotide triads, each of which can code for an amino acid and is termed as a “codon.”

By “heterologous” is meant to occur in a context other than in the naturally occurring (native) context. A “heterologous” polynucleotide sequence indicates that the polynucleotide sequence is being used in a way other than what is found in that sequence's native genome. For example, a “heterologous promoter” is used to drive transcription of a sequence that is not one that is natively transcribed by that promoter; thus, a “heterologous promoter” sequence is often included in an expression construct by means of recombinant nucleic acid techniques. The term “heterologous” is also used to refer to a given sequence that is placed in a non-naturally occurring relationship to another sequence; for example, a heterologous coding or non-coding nucleotide sequence is commonly inserted into a genome by genomic transformation techniques, resulting in a genetically modified or recombinant genome.

As used herein “increasing fitness” or “promoting fitness” of a subject refers to any favorable alteration in physiology, or of any activity carried out by a subject organism, as a consequence of administration of a peptide or polypeptide described herein, including, but not limited to, any one or more of the following desired effects: (1) increased tolerance of biotic or abiotic stress; (2) increased yield or biomass; (3) modified flowering time; (4) increased resistance to pests or pathogens; (4) increased resistance to herbicides; (5) increasing a population of a subject organism (e.g., an agriculturally important insect); (6) increasing the reproductive rate of a subject organism (e.g., insect, e.g., bee or silkworm); (7) increasing the mobility of a subject organism (e.g., insect, e.g., bee or silkworm); (8) increasing the body weight of a subject organism (e.g., insect, e.g., bee or silkworm); (9) increasing the metabolic rate or activity of a subject organism (e.g., insect, e.g., bee or silkworm); (10) increasing pollination (e.g., number of plants pollinated); (11) increasing production of subject organism (e.g., insect, e.g., bee or silkworm) byproducts (e.g., honey from honeybee or silk from a silkworm); (12) increasing nutrient content of the subject organism (e.g., insect) (e.g., protein, fatty acids, or amino acids); (13) increasing a subject organism's resistance to pesticides (e.g., a neonicotinoid (e.g., imidacloprid) or an organophosphorus insecticide (e.g., a phosphorothioate, e.g., fenitrothion); or (14) increasing health or reducing disease of a subject organism such as a human or non-human animal. An increase in host fitness can be determined in comparison to a subject organism to which the modulating agent has not been administered. Conversely, “decreasing fitness” of a subject refers to any unfavorable alteration in physiology, or of any activity carried out by a subject organism, as a consequence of administration of a peptide or polypeptide described herein, including, but not limited to, any one or more of the following intended effects: (1) decreased tolerance of biotic or abiotic stress; (2) decreased yield or biomass; (3) modified flowering time; (4) decreased resistance to pests or pathogens, (4) decreased resistance to herbicides; (5) decreasing a population of a subject organism (e.g., an agriculturally important insect); (6) decreasing the reproductive rate of a subject organism (e.g., insect, e.g., bee or silkworm); (7) decreasing the mobility of a subject organism (e.g., insect, e.g., bee or silkworm); (8) decreasing the body weight of a subject organism (e.g., insect, e.g., bee or silkworm); (9) decreasing the metabolic rate or activity of a subject organism (e.g., insect, e.g., bee or silkworm); (10) decreasing pollination (e.g., number of plants pollinated in a given amount of time) by a subject organism (e.g., insect, e.g., bee or silkworm); (11) decreasing production of subject organism (e.g., insect, e.g., bee or silkworm) byproducts (e.g., honey from a honeybee or silk from a silkworm); (12) decreasing nutrient content of the subject organism (e.g., insect) (e.g., protein, fatty acids, or amino acids); (13) decreasing a subject organism's resistance to pesticides (e.g., a neonicotinoid (e.g., imidacloprid) or an organophosphorus insecticide (e.g., a phosphorothioate, e.g., fenitrothion)); or (14) decreasing health or reducing disease of a subject organism such as a human or non-human animal. A decrease in host fitness can be determined in comparison to a subject organism to which the modulating agent has not been administered. It will be apparent to one of skill in the art that certain changes in the physiology, phenotype, or activity of a subject, e.g., modification of flowering time in a plant, can be considered to increase fitness of the subject or to decrease fitness of the subject, depending on the context (e.g., to adapt to a change in climate or other environmental conditions). For example, a delay in flowering time (e.g., about 10%, 20%, 30%, 40%, 50%, 60%, 70%, 80%, 90%, 95%, 99%, 100% fewer plants in a population flowering at a given calendar date) can be a beneficial adaptation to later or cooler Spring times and thus be considered to increase a plant's fitness; conversely, the same delay in flowering time in the context of earlier or warmer Spring times can be considered to decrease a plant's fitness.

As used herein, the term “intron fragment” refers to a portion of an intron, where a first intron fragment and a second intron fragment together form an intron, such as a catalytic intron. An intron fragment may be a 5′ portion of an intron (e.g., a 5′ portion of a catalytic intron) or a 3′ portion of an intron (e.g., a 3′ portion of a catalytic intron), such that the 5′ intron fragment and the 3′ intron fragment, together, form a functional intron, such as a functional intron capable of catalytic self-splicing. The term intron fragment is meant to refer to an intron split into two portions. The term intron fragment is not meant to state, imply, or suggest that the two portion or halves are equal in length. The term intron fragment is used synonymously with the term split-intron.

As used herein, the term “impurity” is an undesired substance present in a composition, e.g., a pharmaceutical composition as described herein. In some embodiments, an impurity is a process-related impurity. In some embodiments, an impurity is a product-related substance other than the desired product in the final composition, e.g., other than the active drug ingredient, e.g., circular polyribonucleotide, as described herein. As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein, e.g., fragments of 10, 9, 8, 7, 6, 5, or 4 ribonucleic acids, monoribonucleic acids, diribonucleic acids, or triribonucleic acids.

As used herein, the terms “linear polyribonucleotide,” “linear RNA,” and “linear polyribonucleotide molecule” are used interchangeably and mean a polyribonucleotide molecule having a 5′ and 3′ end. One or both of the 5′ and 3′ ends may be free ends or joined to another moiety. A linear polyribonucleotide may be a polyribonucleotide that has not undergone circularization (e.g., is pre-circularized) and can be used as a starting material for circularization through, for example, splint ligation, or chemical, enzymatic, ribozyme- or splicing-catalyzed circularization methods.

As used herein, the term “modified oligonucleotide” means an oligonucleotide containing a nucleotide with at least one modification to the sugar, nucleobase, or internucleotide linkage.

As used herein, the term “modified ribonucleotide” means a ribonucleotide containing a nucleoside with at least one modification to the sugar, nucleobase, or internucleoside linkage.

As used herein, the term “naked delivery” is a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that includes a circular polyribonucleotide without covalent modification and is free from a carrier.

As used herein, the terms “nicked RNA” or “nicked linear polyribonucleotide” or “nicked linear polyribonucleotide molecule” are used interchangeably and mean a polyribonucleotide molecule having a 5′ and 3′ end that results from nicking or degradation of a circular RNA. A “nicked circular RNA” means a circular RNA that has been nicked.

The term “optionally substituted X,” as used herein, is intended to be equivalent to “X, wherein X is optionally substituted” (e.g., “alkyl, wherein said alkyl is optionally substituted”). It is not intended to mean that the feature “X” (e.g., alkyl) per se is optional. The term “optionally substituted,” as used herein, refers to having 0, 1, or more substituents (e.g., 0-25, 0-20, 0-10, or 0-5 substituents). For example, a C₁ alkyl group, i.e., methyl, may be substituted with oxo to form a formyl group and further substituted with —OH or —NH₂ to form a carboxyl group or an amido group.

The term “pharmaceutical composition” is intended to also disclose that the circular or linear polyribonucleotide included within a pharmaceutical composition can be used for the treatment of the human or animal body by therapy.

The term “polynucleotide” as used herein means a molecule including one or more nucleic acid subunits, or nucleotides, and can be used interchangeably with “nucleic acid” or “oligonucleotide.” A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides, ribonucleic acids, or RNA, can refer to macromolecules that include multiple ribonucleotides that are polymerized by way of phosphodiester bonds. Deoxyribonucleotides are nucleotides in which the sugar is deoxyribose.

Polydeoxyribonucleotides, deoxyribonucleic acids, and DNA mean macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, which include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. Embodiments of polynucleotides include isolated and purified DNA/RNA molecules, synthetic DNA/RNA molecules, and synthetic DNA/RNA analogs.

Embodiments of polynucleotides, e.g., polyribonucleotides or polydeoxyribonucleotides, include polynucleotides that contain one or more nucleotide variants, including nonstandard nucleotide(s), non-natural nucleotide(s), nucleotide analog(s) or modified nucleotides. Examples of modified nucleotides include, but are not limited to diamino purine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), butoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). In some embodiments, nucleic acid molecules are modified at the base moiety (e.g., at one or more atoms that typically are available to form a hydrogen bond with a complementary nucleotide or at one or more atoms that are not typically capable of forming a hydrogen bond with a complementary nucleotide), sugar moiety or phosphate backbone. In some embodiments, nucleic acid molecules contain amine-modified groups, such as amino allyl 1-dUTP (aa-dUTP) and aminohexyl acrylamide-dCTP (aha-dCTP) to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo or amplification synthesis are described in Betz K, Malyshev D A, Lavergne T, Welte W, Diederichs K, Dwyer T J, Ordoukhanian P, Romesberg F E, Marx A. NAT. CHEM. BIOL. 2012; 8:612-4, which is herein incorporated by reference for all purposes.

As used herein, the term “polyribonucleotide cargo” herein includes any sequence including at least one polyribonucleotide. In embodiments, the polyribonucleotide cargo includes one or multiple expression (or coding) sequences, wherein each expression (or coding) sequence encodes a polypeptide. In embodiments, the polyribonucleotide cargo includes one or multiple noncoding sequences, such as a polyribonucleotide having regulatory or catalytic functions. In embodiments, the polyribonucleotide cargo includes a combination of expression and noncoding sequences. In embodiments, the polyribonucleotide cargo includes one or more polyribonucleotide sequence described herein, such as one or multiple regulatory elements, internal ribosomal entry site (IRES) elements, or spacer sequences.

As used interchangeably herein, the terms “polyA” and “polyA sequence” refer to an untranslated, contiguous region of a nucleic acid molecule of at least 5 nucleotides in length and consisting of adenosine residues. In some embodiments, a polyA sequence is at least 10, at least 15, at least 20, at least 30, at least 40, or at least 50 nucleotides in length. In some embodiments, a polyA sequence is located 3′ to (e.g., downstream of) an open reason frame (e.g., an open reading frame encoding a polypeptide), and the polyA sequence is 3′ to a termination element (e.g., a Stop codon) such that the polyA is not translated. In some embodiments, a polyA sequence is located 3′ to a termination element and a 3′ untranslated region.

As used herein, the elements of a nucleic acid are “operably connected” or “operably linked” if they are positioned in the vector such that they can be transcribed to form a linear polyribonucleotide that can then be circularized into a circular polyribonucleotide using the methods provided herein.

As used herein, the term “plant-modifying polypeptide” refers to a polypeptide that can alter the genetic properties (e.g., increase gene expression, decrease gene expression, or otherwise alter the nucleotide sequence of DNA or RNA), epigenetic properties, or biochemical or physiological properties of a plant in a manner that results in a change in the plant's physiology or phenotype, e.g., an increase or a decrease in plant fitness.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the terms “purify,” “purifying,” and “purification” refer to one or more steps or processes of removing impurities (e.g., a process-related impurity (e.g., an enzyme), a process-related substance (e.g., a deoxyribonucleotide fragment, a deoxyribonucleotide monomer)) or by-products (e.g., linear RNA) from a sample containing a mixture circular RNA and linear RNA, among other substances, to produce a composition containing an enriched population of circular RNA with a reduced level of an impurity (e.g., a process-related impurity (e.g., an enzyme), a process-related substance (e.g., deoxyribonucleotide fragment, deoxyribonucleotide monomer)) or by-product (e.g., linear RNA) as compared to the original mixture or in which the linear RNA or substances have been reduced by 40% or more by mass (e.g., 50%, 60%, 70%, 80%, 85%, 90%, 95%, 97%, or 99% or more) relative to a starting mixture.

As used herein, the terms “pure” and “purity” refer to the extent to which an analyte (e.g., circular RNA) has been isolated and is free of other components. In the context of nucleic acids (e.g., polyribonucleotides), purity of an isolated nucleic acid (e.g., circular RNA) can be expressed with regard to the population of nucleic acids that is free of any contaminants, impurities, or by-products (e.g, linear RNA, and other substances). For example, purity of a population of circular RNA indicates how much of the population is circular RNA by total mass of the isolated material, which may be determined using, e.g., pure circular RNA as a reference. A level of purity found in the disclosure can be 5%, 10%, 15%, 20%, 25%, 30%, 35%, 40%, 45%, 50%, 55%, 60%, 65%, 70%, 75%, 80%, 85%, 90%, 95%, greater than 95%, or greater than 99% (w/w). In some embodiments, the level of contaminants or impurities or by-products is no more than about 20%, 15%, 12%, 10%, 9%, 8%, 7%, 6%, 5%, 4%, 3%, 2%, or 1% (w/w). Purity can be determined by detecting a level of a specific analyte (e.g., circular RNA) or a specific impurity or by-product (e.g., linear RNA) using gel electrophoresis, spectrophotometry (e.g., NanoDrop by ThermoFisher Scientific), or other technique suitable for measuring purity of a population of nucleic acids and calculating a percentage of the analyte (w/w) relative to the total nucleic acid content (e.g., as determined by an assay known in the art).

As used herein, the phrase “substantially free of one or more impurities or by-products” refers to a property of a sample, such as a sample containing an enriched population of circular RNA, that is free of one or more impurities or by-products (e.g., one or more impurities or by-products disclosed herein) or contains a minimal amount of the one or more impurities or by-products. A minimal amount of the one or more impurities or by-products may be no more than 20% (w/w) (e.g., no more than 19%, 18%, 17%, 16%, 15%, 14%, 13%, 12%, 11%, 10%, 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 15% (w/w) (e.g., no more than 14%, 13%, 12%, 11%, 10%, 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 10% (w/w) (e.g., no more than 9%, 8%, 7%, 6%, 5%, 4%, 3%, 2%, 1% (w/w), or less). In another example, the sample or the enriched population of circular RNA is substantially free of one or more impurities or by-products if the one or more impurities or by-products are present in an amount that is less than 5% (w/w) (e.g., no more than 4%, 3%, 2%, 1% (w/w) or less). In yet another example, the sample or the enriched population of circular RNA is substantially free of one or more impurities or by-products if the one or more impurities or by-products are present in an amount that is less than 1% (no more than 0.9%, 0.8%, 0.7%, 0.6%, 0.5%, 0.4%, 0.3%, 0.2%, 0.1% (w/w), or less).

As used herein, a “regulatory element” is a moiety, such as a nucleic acid sequence, that modifies expression of an expression sequence within the circular or linear polyribonucleotide.

As used herein, the term “replication element” is a sequence and/or motif useful for replication or that initiates transcription of the circular polyribonucleotide.

As used herein, a “spacer” refers to any contiguous nucleotide sequence (e.g., of one or more nucleotides) that provides distance or flexibility between two adjacent polynucleotide regions.

As used herein, the term “sequence identity” is determined by alignment of two peptide or two nucleotide sequences using a global or local alignment algorithm. Sequences are referred to as “substantially identical” or “essentially similar” when they share at least a certain minimal percentage of sequence identity when optimally aligned (e.g., when aligned by programs such as GAP or BESTFIT using default parameters). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides, the default scoring matrix used is nwsgapdna, and for proteins the default scoring matrix is Blosum62 (Henikoff & Henikoff, 1992, PNAS 89, 915-919). Sequence alignments and scores for percentage sequence identity are determined, e.g., using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity is determined by searching against databases, e.g., using algorithms such as FASTA, BLAST, etc. Sequence identity refers to the sequence identity over the entire length of the sequence.

As used herein, “structured” with regard to RNA refers to an RNA sequence that is predicted by the RNAFold software or similar predictive tools to form a structure (e.g., a hairpin loop) with itself or other sequences in the same RNA molecule.

As used herein, the term “subject” refers to an organism, such as an animal, plant, or microbe. In embodiments, the subject is a vertebrate animal (e.g., mammal, bird, fish, reptile, or amphibian). In embodiments, the subject is a human. In embodiments, the subject is a non-human mammal. In embodiments, the subject is a non-human mammal such as a non-human primate (e.g., monkeys, apes), ungulate (e.g., cattle, buffalo, bison, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g, insects, arachnids, crustaceans), a nematode, an annelid, a helminth, or a mollusk. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

As used herein, a “termination element” is a moiety, such as a nucleic acid sequence, that terminates translation of the expression sequence in the circular or linear polyribonucleotide.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules.

As used herein, the terms “treat” and “treating” refer to a prophylactic or therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, or preventing the spread of the disease or disorder as compared to the state or the condition of the disease or disorder in the absence of the therapeutic treatment. Embodiments include treating plants to control a disease or adverse condition caused by or associated with an invertebrate pest or a microbial (e.g., bacterial, fungal, or viral) pathogen. Embodiments include treating a plant to increase the plant's innate defense or immune capability to tolerate pest or pathogen pressure.

As used herein, the term “translation initiation sequence” is a nucleic acid sequence that initiates translation of an expression sequence in the circular or linear polyribonucleotide.

As used herein, a “therapeutic polypeptide” refers to a polypeptide that when administered to or expressed in a subject provides some therapeutic benefit. In embodiments, a therapeutic polypeptide is used to treat or prevent a disease, disorder, or condition in a subject by administration of the therapeutic peptide to a subject or by expression in a subject of the therapeutic polypeptide. In alternative embodiments, a therapeutic polypeptide is expressed in a cell and the cell is administered to a subject to provide a therapeutic benefit.

As used herein, a “vector” means a piece of DNA, that is synthesized (e.g., using PCR), or that is taken from a virus, plasmid, or cell of a higher organism into which a foreign DNA fragment can be or has been inserted for cloning or expression purposes. In some embodiments, a vector can be stably maintained in an organism. A vector can include, for example, an origin of replication, a selectable marker or reporter gene, such as antibiotic resistance or GFP, or a multiple cloning site (MCS). The term includes linear DNA fragments (e.g., PCR products, linearized plasmid fragments), plasmid vectors, viral vectors, cosmids, bacterial artificial chromosomes (BACs), yeast artificial chromosomes (YACs), and the like. In one embodiment, the vectors provided herein include a multiple cloning site (MCS). In another embodiment, the vectors provided herein do not include an MCS.

As used herein, “translation efficiency” is a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., a cell-free translation system like rabbit reticulocyte lysate.

As used herein, the term “yield” refers to the relative amount of an analyte (e.g., a population of circular polyribonucleotides) obtained after a purification step or process as compared to the amount of analyte in the starting material (e.g., a mixed population of polyribonucleotides, such as, e.g., circular, and linear polyribonucleotides) (w/w). The yield may be expressed as a percentage. In the context of the disclosure, the amount of analyte (e.g., circular polyribonucleotides) in the starting material and analyte obtained after the purification step can be measured using an assay (e.g., gel electrophoresis or spectrophotometry). The methods of the disclosure can be used to produce a yield of an enriched population of circular polyribonucleotides of about 20% (w/w) or greater relative to the amount present in the starting material, e.g., mixed population of polyribonucleotides. For example, the methods can be used to produce a yield of purified circular polyribonucleotides of about 25%, 30%, 35%, 40%, 45%, 50%, 55%, 65%, 70%, 75%, 80%, 85%, or 90% (w/w) or greater.

### BRIEF DESCRIPTION OF THE DRAWINGS

FIG. 1 is a schematic drawing showing an exemplary method of separating a circular polyribonucleotide from a linear polyribonucleotide, where the linear polyribonucleotide includes an aptamer. On the left is a linear polyribonucleotide that contains an aptamer located near the 5′ end of the polyribonucleotide, although the disclosure specifically contemplates an alternate embodiment where the aptamer is located at the 3′ end of the linear polyribonucleotide. The linear polyribonucleotide is circularized thereby producing a circular polyribonucleotide that does not include the aptamer. A reagent conjugated to a particle is added to the mixture. The reagent binds to the aptamer on the linear polyribonucleotides while the circular polyribonucleotides are not bound by the reagent, thereby separating the linear polyribonucleotides containing the aptamer from the circular polyribonucleotides that lack the aptamer.

FIG. 2 is a schematic drawing showing an exemplary method of separating a circular polyribonucleotide from a linear polyribonucleotide where the linear polyribonucleotide includes a region that hybridizes to an aptamer. On the left is a linear polyribonucleotide that contains a region located near the 5′ end of the polyribonucleotide that hybridizes to a polyribonucleotide containing an aptamer although the disclosure specifically contemplates an alternate embodiment where the aptamer is hybridized to a location located at the 3′ end of the linear polyribonucleotide. The linear polyribonucleotide is circularized thereby producing a circular polyribonucleotide that does not include the region that hybridizes to the polyribonucleotide containing the aptamer. A reagent conjugated to a particle is added to the mixture. The reagent binds to the aptamer that is hybridized to the linear polyribonucleotides while the circular polyribonucleotides are not bound by the reagent, thereby separating the linear polyribonucleotides hybridized to the aptamer from the circular polyribonucleotides that are not hybridized to the aptamer.

FIG. 3 is a gel showing linear byproducts in an in vitro transcription (IVT) mixture in which circular RNA was generated by self-splicing. The gel shows the desired circular RNA product, unspliced linear RNA, partly spliced linear RNA, nicked circular RNA, and spliced introns.

### DETAILED DESCRIPTION

The present disclosure describes compositions and methods for processing, e.g., purifying, polyribonucleotides. Polyribonucleotides, such as linear or circular polyribonucleotides may be used for a variety of engineering or therapeutic purposes. However, when polyribonucleotides are generated via certain biological reactions, various impurities, byproducts, or incomplete products may be present. The present invention features methods useful to reduce or remove these impurities, byproducts, or incomplete products from a sample to produce compositions with a desired polyribonucleotide composition, amount, and/or purity, or a population containing a plurality of polyribonucleotides with a desired polyribonucleotide composition, amount, and/or purity.

In certain embodiments, the methods are useful for purifying a polyribonucleotide that has undergone a splicing reaction. In such an embodiment, the methods may be used to separate spliced polyribonucleotides from non-spliced polyribonucleotides or non-spliced polyribonucleotides from spliced polyribonucleotides. In some embodiments, the methods may be used to separate circular polyribonucleotides (e.g., that have been spliced) from linear polyribonucleotides or linear polyribonucleotides from circular polyribonucleotides. Such purified compositions containing a desired polyribonucleotide may be useful for various downstream applications, such as delivering a polynucleotide cargo (e.g., encoding a gene or protein) to a target cell. The compositions and methods are described in more detail below.

### Methods

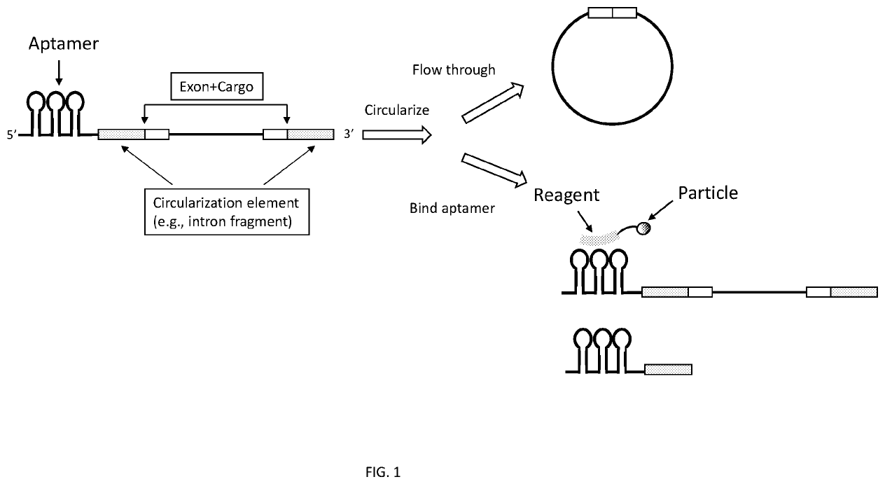

The methods described herein include separating a polyribonucleotide having an aptamer from a plurality of polyribonucleotides. The method includes providing a sample that includes the plurality of polyribonucleotides. The plurality of polyribonucleotides includes a mixture of linear polyribonucleotides and circular polyribonucleotides. A subset of the plurality of polyribonucleotides are linear polyribonucleotides. The method includes contacting the sample with a reagent that binds to the aptamer and separating the linear polyribonucleotide having the aptamer that is bound to the reagent from the plurality of polyribonucleotides in the sample (FIG. 1).

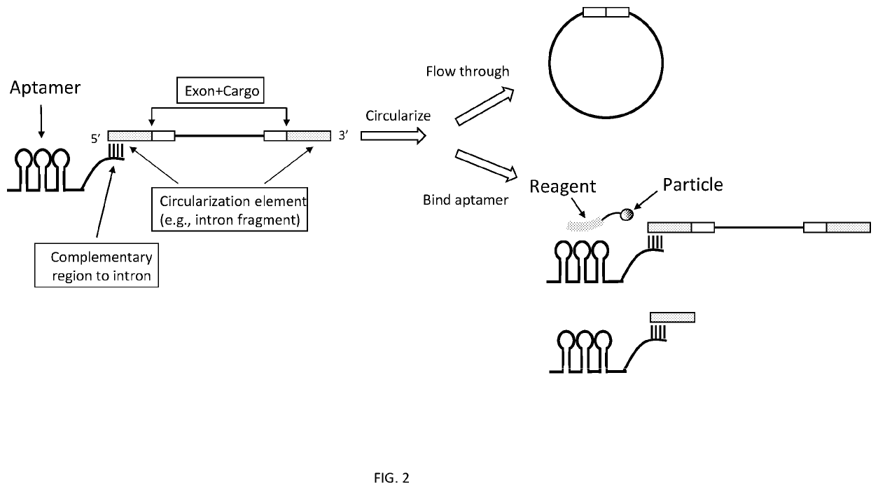

In some embodiments, the method includes producing the linear polyribonucleotide with the aptamer by attaching the aptamer to the linear polyribonucleotide (FIG. 2). The circular polyribonucleotides may include an open reading frame (ORF) encoding a polypeptide.

In some embodiments, the methods described herein include separating a linear polyribonucleotide from a plurality of polyribonucleotides. The plurality of polyribonucleotides include a mixture of linear polyribonucleotides and circular polyribonucleotides. The method includes providing a sample with the plurality of polyribonucleotides, wherein a subset of the plurality of polyribonucleotides include the linear polyribonucleotide; attaching an aptamer to the linear polyribonucleotide; and contacting the sample with a column that includes a resin with a plurality of particles conjugated to a reagent that binds to the aptamer. The method further includes collecting an eluate that includes a portion of the sample that is not bound to the reagent from the plurality of polyribonucleotides in the sample.

In some embodiments, the methods described herein include separating a linear polyribonucleotide from a plurality of polyribonucleotides. The plurality of polyribonucleotides include a mixture of linear polyribonucleotides and circular polyribonucleotides. The method includes circularizing a linear precursor to form the circular polyribonucleotide; providing a sample that includes the plurality of polyribonucleotides, wherein a subset of the plurality of polyribonucleotides include the linear polyribonucleotide; attaching an aptamer to the linear polyribonucleotide; and contacting the sample with a reagent that binds to the aptamer. The method further includes separating the linear polyribonucleotide with the aptamer that is bound to the reagent from the plurality of polyribonucleotides in the sample.

In some embodiments of any of the methods described herein, the aptamer is located at a 5′ or 3′ terminus of the polyribonucleotide (e.g., linear or circular polyribonucleotide). In some embodiments, the aptamer is located at a 3′ terminus of the polyribonucleotide. In some embodiments, the aptamer does not contain a polyA sequence.

In some embodiments, the reagent is conjugated (e.g., directly, or indirectly) to a particle. The particle may be, for example, a magnetic bead. In some embodiments, the reagent is conjugated to a resin that includes a plurality of the particles. The resin may include, for example, cross-linked poly[styrene-divinylbenzene], agarose, or SEPHAROSE®. In some embodiments, a column includes the resin.

In some embodiments of any of the methods described herein, separating includes immobilizing the reagent. The method may include, for example, immobilizing the reagent, the particle, or a combination thereof.

In some embodiments, the particle is a magnetic particle. The method may include applying a force to the magnetic particle, such as a magnetic force. The particle or bead may be, e.g., a crosslinked agarose, e.g., SEPHAROSE®, bead. The method may include applying a force to the bead or particle, such as a mechanical, optical, centrifugal, or acoustic force.

As described herein, the methods may be used to separate, e.g., spliced from non-spliced polyribonucleotides. In some embodiments, the methods described herein include separating a spliced polyribonucleotide from a non-spliced or partially spliced polyribonucleotide. In some embodiments, the spliced polyribonucleotide is a circular polyribonucleotide. In some embodiments, the spliced polyribonucleotide is a linear polyribonucleotide. In some embodiments, the spliced polyribonucleotide lacks an intron, e.g., following a splicing (e.g., self-splicing) event during generation. In some embodiments, the polyribonucleotide having the intron is a linear polyribonucleotide.

In some embodiments, the method further includes washing the bound polyribonucleotide having the aptamer one or more (e.g., two, three, four five, or more) times. The washing may occur after the contacting and/or after separating step.

In some embodiments, the method further includes performing a first elution step to release the bound polyribonucleotide with the aptamer from the polyribonucleotide having the aptamer. The first elution step may include adding a first buffer and/or heating the sample, e.g., to at least 50° C., 55° C., 60° C., 65° C., 70° C., 75° C., 80° C., or higher.

In some embodiments, the method further includes performing a second elution step. The 30 second elution step may include adding a second buffer and/or heating the sample, e.g., to at least 50° C., 55° C., 60° C., 65° C., 70° C., 75° C., 80° C., or higher. In some embodiments, the second buffer includes a denaturing agent, e.g., formamide or urea. The second buffer may include, e.g., from about 40% to about 60% formamide (e.g., about 40%, 45%, 50%, 55%, or 60% formamide).

In some embodiments, the method includes incubating the sample with the reagent for at least ten (e.g., at least 20, 30, 40, 50, 60, 70, 80, 90, 100, 110, 120, or more) minutes.

In some embodiments, the method includes collecting a portion of the sample that is not bound by the reagent.

In some embodiments, the method includes providing a plurality of reagents, wherein each reagent binds to a distinct aptamer or a distinct portion within an aptamer. Each reagent may be, e.g., conjugated to a particle, e.g., a magnetic particle or a bead.

In some embodiments, the method includes providing the reagent at a molar ratio of 10:1 to 1:10 (e.g., 10:1, 5:1, 2:1, 1:2, 1:5, or 1:10) to the polyribonucleotide, e.g., polyribonucleotide containing the aptamer.

In some embodiments, the method includes providing a sample of particles, e.g., beads, e.g., magnetic beads. The particles may be present in a vessel, e.g., a microcentrifuge tube, or packed in a column. The particles may be conjugated to the reagent. The method may include flowing the mixture of polyribonucleotides over the column containing the particles. As such, the polyribonucleotides bound by the reagent will bind the column. In some embodiments, the particles are conjugated directly to a reagent, e.g., configured to bind to the aptamer of the polyribonucleotide.

In some embodiments, e.g., when using a magnetic particle, the method may include pelleting the magnetic particles, e.g., in a vessel (e.g., microcentrifuge tube) by providing a permanent magnet.

In some embodiments, the methods described herein enrich an amount of the desired polyribonucleotide in the sample. For example, the method may enrich the amount of the desired (e.g., spliced, e.g., circular) polyribonucleotide by at least 10%, (e.g., at least 20%, 30%, 40%, 50%, 55%, 60%, 65%, 70%, 75%, 80%, 85%, 90%, 95%, 97%, 99%, or more) relative to the sample prior to purification.

In some embodiments, the methods of purification result in a circular polyribonucleotide that has less than 50% (mol/mol) (e.g., less than 40%, 30%, 20%, 10%, 5%, 4%, 3%, 2%, or 1% (mol/mol)) linear polyribonucleotides.

In some embodiments, the methods described herein separate at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more) of the linear polyribonucleotide with the aptamer. In some embodiments, the method separates from 500 μg to 1,000 mg of the linear polyribonucleotide including the aptamer.

### Methods of Attachment

The methods described herein include attaching an aptamer to a linear polynucleotide. For example, the method may include attaching an aptamer to a 3′ or 5′ end of a linear polyribonucleotide. In some embodiments, the method includes attaching an aptamer to a 3′ or 5′ end of a linear polyribonucleotide, and the aptamer is not located at the 3′ or 5′ terminus of the linear polyribonucleotide. In embodiments, a polyribonucleotide containing the aptamer is attached to the terminus and the aptamer is ligated to the terminus of the linear polyribonucleotide while a flanking region forms a new 5′ or 3′ terminus of the linear polyribonucleotide, e.g., after attachment. Attachment may be performed by ligating the aptamer to the linear polyribonucleotide. In some embodiments, the linear polyribonucleotide contains a portion of the aptamer, and the attachment step includes attaching the remaining part of the aptamer.

The aptamer or a polyribonucleotide containing an aptamer may be attached according to any available technique, including, but not limited to chemical methods and enzymatic methods.

Such enzymatic methods include, for example, providing a ligase (e.g., RNA ligase), which attaches free ends of linear RNA, e.g., a 3′ end of the linear polyribonucleotide and a 5′ end of the aptamer or a 5′ end of the linear polyribonucleotide and a 3′ end of the aptamer.

In one example, either the 5′ or 3′ end of the linear polyribonucleotide can encode a ligase ribozyme sequence such that during in vitro transcription, the resultant linear polyribonucleotide includes an active ribozyme sequence capable of ligating the 5′ end of the linear polyribonucleotide or the 3′ end of the linear polyribonucleotide to the aptamer. The ligase ribozyme may be derived from the Group I Intron, Hepatitis Delta Virus, Hairpin ribozyme or may be selected by SELEX (systematic evolution of ligands by exponential enrichment).

In another example, an aptamer may be attached to the linear polyribonucleotide using at least one non-nucleic acid moiety. For example, the at least one non-nucleic acid moiety may react with regions or features near the 5′ terminus or near the 3′ terminus of the linear polyribonucleotide in order to attach to the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety is a linkage such as a hydrophobic linkage, ionic linkage, a biodegradable linkage, or a cleavable linkage. As another non-limiting example, the non-nucleic acid moiety is a ligation moiety. As yet another non-limiting example, the non-nucleic acid moiety is an oligonucleotide or a peptide moiety, such as an aptamer or a non-nucleic acid linker as described herein.

In another example, the linear polyribonucleotide may be spliced to the aptamer. In some embodiments, the linear polyribonucleotide and the aptamer together include loop E sequence to ligate. In another embodiment, the linear polyribonucleotide and the aptamer include a circularizing intron, e.g., a 5′ and 3′ slice junction, or a circularizing catalytic intron such as a Group I, Group II, or Group Ill Introns. Nonlimiting examples of group I intron self-splicing sequences include self-splicing permuted intron-exon sequences derived from T4 bacteriophage gene td, and the intervening sequence (IVS) rRNA of Tetrahymena.

In another example, an aptamer may be attached to the linear polyribonucleotide by a non-nucleic acid moiety that causes an attraction between atoms, molecular surfaces at, near, or linked to the 5′ and 3′ ends of the linear polyribonucleotide. The linear polyribonucleotide may be attached to the aptamer by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may include a ribozyme RNA sequence near the 5′ terminus and the aptamer may include a ribozyme RNA sequence near the 3′ terminus, or vice versa. The ribozyme RNA sequence may covalently link to a peptide when the sequence is exposed to the remainder of the ribozyme. The peptides covalently linked to the ribozyme RNA sequence near the 5′ terminus and the 3 ′ terminus may associate with each other, thereby attaching the aptamer to the linear polyribonucleotide. Non-limiting examples of ribozymes for use in the linear primary constructs or linear polyribonucleotides of the present invention or a non-exhaustive listing of methods to incorporate or covalently link peptides are described in US patent application No. US20030082768, the contents of which is here in incorporated by reference in its entirety.

In yet another example, chemical methods of ligation may be used to attach the aptamer to the linear polyribonucleotide. Such methods may include, but are not limited to click chemistry (e.g., alkyne and azide-based methods, or clickable bases), olefin metathesis, phosphoramidate ligation, hemiaminal-imine crosslinking, base modification, and any combination thereof.

### Methods of Circularization

The disclosure provides methods of circularization of a polyribonucleotide, e.g., from a linear precursor. Circularization may be performed using methods including, e.g., recombinant technology or chemical synthesis. For example, a DNA molecule used to produce an RNA circle can include a DNA sequence of a naturally occurring original nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

The circular polyribonucleotides may be prepared according to any available technique, including, but not limited to chemical synthesis and enzymatic synthesis. In some embodiments, a linear primary construct or linear RNA may be cyclized or concatenated to create a circRNA described herein. The mechanism of cyclization or concatenation may occur through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, can be used for splint ligation. According to this method, a single stranded polynucleotide (splint), such as a single-stranded DNA or RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circRNA. In some embodiments, a DNA or RNA ligase may be used in the synthesis of the circular polynucleotides. As a non-limiting example, the ligase may be a circ ligase or circular ligase.

In another example, either the 5′ or 3′ end of the linear polyribonucleotide can encode a ligase ribozyme sequence such that during in vitro transcription, the resultant linear circRNA includes an active ribozyme sequence capable of ligating the 5′ end of the linear polyribonucleotide to the 3′ end of the linear polyribonucleotide. The ligase ribozyme may be derived from the Group I Intron, Hepatitis Delta Virus, Hairpin ribozyme or may be selected by SELEX (systematic evolution of ligands by exponential enrichment).

In another example, a linear polyribonucleotide may be cyclized or concatenated by using at least one non-nucleic acid moiety. For example, the at least one non-nucleic acid moiety may react with regions or features near the 5′ terminus or near the 3′ terminus of the linear polyribonucleotide in order to cyclize or concatenate the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety may be a linkage such as a hydrophobic linkage, ionic linkage, a biodegradable linkage or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, linear polyribonucleotides may be cyclized or concatenated by self-splicing. In some embodiments, the linear polyribonucleotides may include loop E sequence to self-ligate. In another embodiment, the linear polyribonucleotides may include a self-circularizing intron, e.g., a 5′ and 3′ slice junction, or a self-circularizing catalytic intron such as a Group I, Group II or Group III Introns. Nonlimiting examples of group I intron self-splicing sequences may include self-splicing permuted intron-exon sequences derived from T4 bacteriophage gene td, the intervening sequence (IVS) rRNA of Tetrahymena, or a cyanobacterium Anabaena pre-tRNA-Leu gene.

In another example, a linear polyribonucleotide may be cyclized or concatenated by a non-nucleic acid moiety that causes an attraction between atoms, molecular surfaces at, near, or linked to the 5′ and 3′ ends of the linear polyribonucleotide. The one or more linear polyribonucleotides may be cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may include a ribozyme RNA sequence near the 5′ terminus and near the 3′ terminus. The ribozyme RNA sequence may covalently link to a peptide when the sequence is exposed to the remainder of the ribozyme. The peptides covalently linked to the ribozyme RNA sequence near the 5′ terminus and the 3′ terminus may associate with each other, thereby causing a linear polyribonucleotide to cyclize or concatenate. In another example, the peptides covalently linked to the ribozyme RNA near the 5′ terminus and the 3′ terminus may cause the linear primary construct or linear mRNA to cyclize or concatenate after being subjected to ligated using various methods known in the art such as, but not limited to, protein ligation. Non-limiting examples of ribozymes for use in the linear primary constructs or linear polyribonucleotides of the present invention or a non-exhaustive listing of methods to incorporate or covalently link peptides are described in US patent application No. US20030082768, the contents of which is here in incorporated by reference in its entirety.

In yet another example, chemical methods of circularization may be used to generate the circular polyribonucleotide. Such methods may include, but are not limited to click chemistry (e.g., alkyne and azide-based methods, or clickable bases), olefin metathesis, phosphoramidate ligation, hemiaminal-imine crosslinking, base modification, and any combination thereof.

Methods of making the circular polyribonucleotides described herein are described in, for example, Khudyakov & Fields, ARTIFICIAL DNA: METHODS AND APPLICATIONS, CRC Press (2002); in Zhao, SYNTHETIC BIOLOGY: TOOLS AND APPLICATIONS, (First Edition), Academic Press (2013); and Egli & Herdewijn, CHEMISTRY AND BIOLOGY OF ARTIFICIAL NUCLEIC ACIDS, (First Edition), Wiley-VCH (2012).

Various methods of synthesizing circular polyribonucleotides are also described elsewhere (see, e.g., U.S. Pat. Nos. 6,210,931, 5,773,244, 5,766,903, U.S. Pat. Nos. 5,712,128, 5,426,180, US Publication No. US20100137407, International Publication No. W01992001813, International Publication No. W02010084371, and Petkovic et al., NUCLEIC ACIDS RES. 43:2454-65 (2015); the contents of each of which are herein incorporated by reference in their entirety).

### Reagents

The methods described herein employ a reagent that binds to an aptamer on a polyribonucleotide. The reagent may be, for example, a polypeptide, a small molecule, a lipid, a carbohydrate, an RNA, or a metal.

In some embodiments, the reagent is a polypeptide. The polypeptide may be, for example, Protein A, streptavidin, lambda peptide, or MS2 bacteriophage coat protein. In some embodiments, the polypeptide is a polypeptide selected from Table 1.

In some embodiments, the reagent is a small molecule. The small molecule may be, for example, a small molecule selected from Table 2. In some embodiments, the small molecule is biotin or tetracycline. In some embodiments, the small molecule is a metabolite or an amino acid.

In some embodiments, the reagent is a carbohydrate.

In some embodiments, the reagent is a lipid.

In some embodiments, the reagent is an RNA. In some embodiments, the RNA is selected from Table 3.

In some embodiments, the reagent is a metal. The metal may be, for example, nickel, cobalt, cadmium, zinc, or manganese. In some embodiments, the metal is selected from Table 4.

### Aptamers

The aptamer of a polyribonucleotide as described herein is configured to bind to a reagent. In some embodiments the aptamer may contain modified nucleotides (e.g., having modified phosphate, sugar, or base). In some embodiments, the aptamer contains a portion configured to bind to the reagent and a portion that does not bind to the reagent (e.g., a terminal region).

The aptamer may be, for example, at least 5 nucleotides (e.g., at least 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides) in length. In some embodiments, the aptamer is, e.g., from 5-200, 10-200, 10-150, 10-100, 10-50, 20-200, 20-150, 20-100, 20-50, 5-100, 5-95, 10-90, 10-80, 12-60, 15-50, 15-40, 15-30, 18-30, 20-25, or 20-22 nucleotides in length.

The aptamer may have, for example, a GC content of from 30-70%, e.g., 30%, 35%, 40%, 45%, 50%, 55%, 60%, 65%, or 70%. The aptamer may have a melting temperature (Tm) of, for example, from about 45° C. to about 75° C., e.g., about 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., or 75° C.

In some embodiments, the aptamer includes a nucleic acid sequence selected from any one of SEQ ID NOs: 1-124. For example, the aptamer may include a nucleic acid sequence having at least 85% (e.g., at least 90%, 95%, 97%, 99%, or 100%) sequence identity to any one of SEQ ID NOs: 1-124.

In some embodiments, the aptamer may include a nucleic acid sequence having at least 85% (e.g., at least 90%, 95%, 97%, 99%, or 100%) sequence identity to an aptamer as shown in Table 1. The reagent may be a corresponding reagent as shown in Table 1 (e.g., any one of SEQ ID NOs: 1-66).

In some embodiments, the aptamer may include a nucleic acid sequence having at least 85% (e.g., at least 90%, 95%, 97%, 99%, or 100%) sequence identity to an aptamer as shown in Table 2 (e.g., any one of SEQ ID NOs: 67-119). The reagent may be a corresponding reagent as shown in Table 2.

In some embodiments, the aptamer may include a nucleic acid sequence having at least 85% (e.g., at least 90%, 95%, 97%, 99%, or 100%) sequence identity to an aptamer as shown in Table 3. The reagent may be a corresponding reagent as shown in Table 3 (e.g., SEQ ID NOs: 120 or 121).

In some embodiments, the aptamer may include a nucleic acid sequence having at least 85% (e.g., at least 90%, 95%, 97%, 99%, or 100%) sequence identity to an aptamer as shown in Table 4. The reagent may be a corresponding reagent as shown in Table 4 (e.g., any one of SEQ ID NOs: 122-124).

TABLE 1
Polypeptide Binding Aptamers

| | | | SEQ | |
|---|---|---|---|---|
| | | | ID | |
| Aptamer | Type | Sequence (5′→3′) | NO: | Reagent |
| Bovine Thrombin | Polypeptide | GCAAUGGUACGGUACUUCCUUU | 1 | Bovine |
| (T7 05 RNA) | | GGAAGAUAGCUGGAGAACUAAC | | Thrombin |
| | | CAAAAGUGCACGCUACUUUGCUA | | |
| | | A | | |
| Hepatitis C NS3 | Polypeptide | GGGAGAGCGGAAGCGUGCUGGG | 2 | Hepatitis C NS3 |
| Protein (10G-1) | | CCAGUAGUGUAUAGGGCUCGAA | | Protein |
| | | AUGUUCAUGGCUCAGUGGACAU | | |
| S. Enterica | Polypeptide | GGGAACAGUCCGAGCCUCACUG | 3 | S. Enterica |
| Serovar Typhi | | UUAUCCGAUAGCAGCGCGGGAU | | Serovar Typhi |
| IVP Pili Protein | | GAGGGUCAAUGCGUCAUAGGAU | | IVP Pili Protein |
| (S-PS8.4) | | CCCGC | | |
| Hepatitis C Virus | Polypeptide | GGGAGAAUUCCGACCAGAAGCU | 4 | Hepatitis C |
| Non-Structural | | UCGGGAUUUGAGGGUAGAAUGG | | Virus Non- |
| Protein 3 (G9-1) | | GACUACCUUUCCUCUCUCCUUC | | Structural |
| | | CUCUUCU | | Protein 3 |
| HIV-1 Integrase | Polypeptide | GGGAGCUCAGAAUAAACGCUCAA | 5 | HIV-1 Integrase |
| (P5) | | CCAGUCUUGUGGCUUUGAAAGA | | |
| | | GAGGAGUGUUCGACAUGAGGCC | | |
| | | CGGAUCCGGC | | |
| HER3 (A30) | Polypeptide | GAAUUCCGCGUGUGCCAGCGAA | 6 | HER3 |
| | | AGUUGCGUAUGGGUCACAUCGC | | |
| | | AGGCACAUGUCAUCUGGGCGGU | | |
| | | CCGUUCGGGAUCC | | |
| Interleukin 32 | Polypeptide | GGGUUCACUGCAGACUUGACGA | 7 | Interleukin-32 |
| (IL-32) (AC3-3) | | AGCUUCCGGAGAGAAGGGUCAA | | (IL-32) |
| | | AGUUGUGCGGGAGUGUGUUGUG | | |
| | | GAAUGGAUCCACAUCUACGAAUU | | |
| | | C | | |
| SARS | Polypeptide | GAUAAUACGACUCACUAUAGGGU | 8 | SARS |
| Coronavirus | | UCACUGCAGACUUGACGAAGCU | | Coronavirus |
| NTPase/Helicase | | UGCAGAAAAGGGGGAAGAAGAG | | NTPase/Helicase |
| (ES15-1) | | GGUGAUUCAGGCGAGAGAAUGG | | |
| | | AUCCACAUCUACGAAUUC | | |
| Colicin E3 (F1-1) | Polypeptide | GGGAGAAUUCCGACCAGAAGCU | 9 | Colicin E3 |
| | | UACUGUCCUCCCUUCAGAGAGC | | |
| | | GCGGGACCCUUAACUUGGGGCC | | |
| | | CACGAACAGCUUCAGUUCCGUC | | |
| | | UCGGCGUCAUAUGUGCGUCUAC | | |
| | | AUGGAUCCUCA | | |
| ppERK2/ERK2 | Polypeptide | GGGAGAGCCAUACCUGACAAAG | 10 | ppERK2/ERK2 |
| | | ACGCUAGCGAAUUGGUUCCUCA | | |
| | | CUCAAAAGUAGGGGAAAGCGUU | | |
| | | AUUAAGAAACCAAAAUUUGACAG | | |
| | | GUUACGCAUCCUGCAUCCUCCA | | |
| | | GGUUACGCAUCC | | |
| Basic Fibroblast | Polypeptide | GGUGAAGGCAACGUAUAGGCAA | 11 | Basic Fibroblast |
| Growth Factor | | GCACACUUCACC | | Growth Factor |
| (26A-t) | | | | (bFGF) |
| HIV-1 | Polypeptide | GGAGACAUCCCUCUUGUUGGUG | 12 | HIV-1 |
| Nuceocapsid | | GUGCCGUGUGGAUGUCUC | | Nuceocapsid |
| Lysozyme | Polypeptide | GGGAAUGGAUCCACAUCUACGA | 13 | Lysozyme |
| | | AUUCAUCAGGGCUAAAGAGGCA | | |
| | | GAGUUACUUAGUUCACUGCAGA | | |
| | | CUUGACGAAGCUU | | |
| C-Reactive | Polypeptide | GCCUGUAAGGUGGUCGGUGUGG | 14 | Monomeric C- |
| Protein Monomer | | CGAGUGUGUUAGGAGAGAUUGC | | Reactive Protein |
| | | | | (CRP) |
| Beta 2-Integrin | Polypeptide | GGGCGCUAAGUCCUCGCUCAUG | 15 | Beta 2-Integrin |
| (D20) | | CGCGUCCCAUGGGGUAUAGAGG | | |
| | | GGUCGAAGUGGACGCGCGACUC | | |
| | | GGAUCCU | | |
| Hepatitis C Virus | Polypeptide | GGGAUGCUUCGGCAUCCCCGAA | 16 | Hepatitis C |
| Dependent RNA | | GCCGCUAUGGACCAGUGGCGCG | | Virus |
| Polymerase (B.2) | | GCUUCGGCCCGACGGAGUGGUA | | Dependent RNA |
| | | CCGCUUCGGCGGUACGUAAGCU | | Polymerase |
| | | UGGG | | |
| NF-kappa B | Polypeptide | GGGAUAUCCUCGAGCAUAAGAAA | 17 | NF-kappa B |
| (Atamer 3) | | CAAGAUAGAUCCUGAAACUGUUU | | |
| | | UAAGGUUGGCCGAUCUUCUGCU | | |
| | | CGAGAAUGCAUGAAGCGUUCCA | | |
| | | UAUUUUU | | |
| Activated Protein | Polypeptide | GUGAGACCAGCCGAGUGGUGUC | 18 | Human |
| C (APC 99) | | UGGCUAUUCACUGGAGCGUGGG | | Activated |
| | | UGGAACCCCUGCGCACUCGUUU | | Protein C |
| | | GGCUGUCCGGGCCUUCGGGCCG | | |
| | | GGAUUAUCUCU | | |
| Protein Tyrosine | Polypeptide | GGGAGAUACCAGCUUAUUCAAU | 19 | Protein tyrosine |
| Phosphatase | | UCUGGCAAUGGGCUAUCCCAAG | | phosphatase |
| (N71yc16) | | UGCUAGGCUUCAGGGAGCGAGG | | (PTPase) |
| | | ACCAGACGACGUACCUAACCCUA | | |
| | | AGGUGAGAUAGUAAGUGCAAUC | | |
| | | U | | |
| ERK 1/ ERK2 | Polypeptide | GGGAGAGCCAUACCUGACAAAG | 20 | Extracellular |
| (Family II) | | ACGCUAGCGAAUUGGUUCCUCA | | Regulated |
| | | CUCAAAAGUAGGGGAAAGCGUU | | Kinase 1 and 2 |
| | | AUUAAGAAACCAAAAUUUGACAG | | (ERK 1 and |
| | | GUUACGCAUCCUGCAUCCUC | | ERK2) |
| Moloney Murine | Polypeptide | UUACCACGCGCUCUUAACUGCU | 21 | Moloney Murine |
| Leukemia Virus | | AGCGCCAUGGC | | Leukemia Virus |
| | | | | (M-MLV) RT |
| Endothelial | Polypeptide | UUCAACGCUGUGAAGGGCUUAU | 22 | Alpha-V Beta-3 |
| Integrin Alpha-V | | ACGAGCGGAUUACCC | | |
| Beta-3 (Clone | | | | |
| 17.16) | | | | |
| Pepocin (9- | Polypeptide | GGGAGUCUGAAGUCGGACUUGU | 23 | Pepocin |
| 41U22) | | UAUCAAUUCACUUCAGACU | | |
| Feline | Polypeptide | GGGAGGAUAUUUUCUCAGACCG | 24 | Feline |
| Immunodeficiency | | UAAGUACCGAAUGUGCUUUUGG | | Immunodeficien |
| Virus (F1a) | | CCGAUUUUUGGCCCCUGCAGUU | | cy Virus (FIV) |
| | | GCAGCAUCGUGAACUAGGAUCC | | |
| | | GGG | | |
| Avian | Polypeptide | CGUCCCGUGCGCAAAAGUUCUU | 25 | Avian |
| Myeloblastosis | | AGCGCUAGCAGUCCUAGUUGC | | Myeloblastosis |
| Virus (a.1.1) | | | | Virus (AMV) RT |
| Hepatitis B Virus | Polypeptide | UGUUCAUGUCCUACUGUUCAAA | 26 | Hepatitis B Virus |
| (HBV) | | CAAAAAAACUGUGCACAAAAAUA | | (HBV) |
| Polymerase (P | | AAUUGGGGCAUGGACA | | Polymerase (P |
| protein) (A9) | | | | protein) |
| TLR3-ECD | Polypeptide | GGUAGAUACGAUGGAUACCCCC | 27 | Toll-like receptor |
| (Family-1) | | UGUGGCCCGUCAACACAGGGGA | | 3 Ectodomain |
| | | AGUGGCAUGACGCGCAGCCA | | |
| H5 Avian | Polypeptide | GGGUUCACUGCAGACUUGACGA | 28 | Hemagglutinin |
| Influenza Virus | | AGCUUACAAACAAGAGCAAAAAG | | of the Subtype |
| (HAS15-5) | | GGAGUUGACGUAGACUGUGCGG | | H5 Avian |
| | | AAUGGAUCCACAUCUACGAAUUC | | Influenza Virus |
| GRK2 (C13.28) | Polypeptide | GGCAGACCAUACGGGAGAGAAA | 29 | G protein- |
| | | CUUGCC | | coupled |
| | | | | receptor kinase |
| | | | | 2 |
| SP6 RNA | Polypeptide | UUGCUUGGAAUGCGUUAUAGUC | 30 | SP6 RNA |
| polymerase (S05) | | UCUUAGGUGUGUA | | Polymerase |
| HIV-1 Tat protein | Polypeptide | ACGAAGCUUGAUCCCGUUUGCC | 31 | HIV-1 Tat |
| | | GGUCGAUCGCUUCGA | | protein |
| Mouse Lipocalin- | Polypeptide | CCUCCGGCUCAUACCUUUUCGA | 32 | Mouse |
| 2 (mLcn2) (Oligo | | AGACAAGCUUCGACAGGAGG | | Lipocalin-2 |
| 569) | | | | (mLcn2) |
| Interleukin 10 | Polypeptide | GGGCUCAUGCACGUUUGCUCCU | 33 | Interleukin -10 |
| (IL-10) Receptor | | GUAAUUGGCGUAUGUAACCCAG | | receptor (IL-10) |
| (R5A1) | | GCACCAAACACCCCAGGCCGGG | | |
| | | CCAUGAUCCACAUA | | |
| Unphosphorylate | Polypeptide | GGCGUGACCUGAUGAGUCACGC | 34 | Unphosphorylat |
| d ERK2 | | UAAGGAGGAUUUCCGAAAGCGG | | ed ERK2 |
| | | CUACGGUCCGCCAGUGUUACGA | | |
| | | AACGUUCCC | | |
| Phosphorylated | Polypeptide | GGCGUGACCUGAUGAGUCACGC | 35 | Phosphorylated |
| ERK2 | | AGACGCUAGCGAAUUGGUUCCU | | ERK2 |
| | | CGAAAGGGGAAAGCGUUAUUAA | | |
| | | GAAACCAAAAUGUGUUACGAAAC | | |
| | | GUUCCC | | |
| Hen Egg White | Polypeptide | GGACCUCGGCGAAAGCUAACGU | 36 | Hen Egg White |
| Lysozyme (cyt7- | | CUCAUGGCUAAAUUGCCAUGUU | | Lysozyme |
| 2) | | GCUACAAAUGAUAUGACUAGAGA | | |
| | | GGUUAGGUGCCUCGUGAUGUCC | | |
| | | AGUCGC | | |
| Signal | Polypeptide | GGGAGACAAGAAUAAACGCUCAA | 37 | Signal |
| Recognition | | CACAGAACGCGGUCCCCACACA | | Recognition |
| Particle 19 | | GGACAGGAGCCAGCCCCGGUUC | | Particle 19 |
| (SRP19) | | GACAGGAGGCUCACAACAGGC | | (SRP19) |
| C-Reactive | Polypeptide | GCCUGUAAGGUGGUCGGUGUGG | 38 | C-Reactive |
| Protein | | CGAGUGUGUUAGGAGAGAUUGC | | Protein (CRP) |
| AMPA GluR2 | Polypeptide | GGGCGAAUUCAACUGCCAUCUA | 39 | AMPA GluR2 |
| (AN58) | | GGCAGUAACCAGGAGUUAGUAG | | Glutamate |
| | | GACAAGUUUCGUCC | | Receptor |
| | | | | Channel |
| Mouse | Polypeptide | GGGCGAAUUCCGCGUGUGCUGA | 40 | Mouse |
| Transferrin | | GGGCGGAAGAACUAAUUUGGGA | | Transferrin |
| Receptor (FB4) | | CGGAUUGCGGCCGUUGUCUGUG | | Receptor |
| | | GCGUCCGUUCGGG | | (mTfR) |
| Anti-NF-kB p65 | Polypeptide | GAAGCUUUCACACAACAAGGCCC | 41 | NF-kB p65 |
| (R2) | | GGGACUGUAUUAGGGAAAUUAG | | |
| | | AGUACAGACAGUCGCCGUGGGU | | |
| | | CGAAUUCCGCUCA | | |
| Anti-NF-kB p65 | Polypeptide | GAAGCUUACAAGAAGGACAGCAC | 42 | NF-kB p65 |
| (R1) | | GAAUAAAACCUGCGUAAAUCCGC | | |
| | | CCCAUUUGUGUAAGGGUAGUGG | | |
| | | GUCGAAUUCCGCUCA | | |
| Anti-NF-kB p65 | Polypeptide | GCAUGCAGUGUCUAUUCUCGAG | 43 | Anti-NF-kB p65 |
| (D1) | | UAGCGAUCGUUGAAGGGGUAUA | | |
| | | AGGUUGGCAGAUCGCUAGCAUG | | |
| | | CAACUGACUCGGAUAAGCA | | |
| Influenza B | Polypeptide | GGGAGCUCAGCCUUCACUGCAC | 44 | Influenza B |
| Hemagglutinin | | UCCGGCUGGUGGACGCGGUACG | | Hemagglutinin |
| (A20) | | AGCAAUUUGUACCGGAUGGAUG | | (HA) |
| | | UUCGGGCAGCGGUGUGGCAGGG | | |
| | | AUGAGCGGCACCACGGUCGGAU | | |
| | | CCAC | | |
| C-Reactive | Polypeptide | GGGCGAAUUCGGGACUUCGAUC | 45 | C-Reactive |
| Protein (CRP1-1) | | CGUAGUACCCACCAGGCAUACAC | | Protein (CRP) |
| | | CAGCACGCGGAGCCAAGGAAAAA | | |
| | | UAGUAAACUAGCACUCAGUGCUC | | |
| | | GUAUGCGGAAGCU | | |
| ERK1/ ERK2 | Polypeptide | GGAAAGACGCUAGCGAAUUGGU | 46 | Extracellular |
| (Family II - | | UCCUCGAAAGGGGAAAGCGUUA | | Regulated |
| Truncated) | | UUAAGAAACCAAAAUUUCC | | Kinase 1 and 2 |
| | | | | (ERK 1 and |
| | | | | ERK2) |
| Catenin | Polypeptide | GGACGCGUGGUACCAGGCCGAU | 47 | Arm 1-12 of B- |
| | | CUAUGGACGCUAUAGGCACACC | | catenin |
| | | GGAUACUUUAACGAUUGGCUAA | | |
| | | GCUUCCGCGGGGAUC | | |
| Interleukin-6 | Polypeptide | GGAAGAAAGAGGUCUGAGACAU | 48 | Soluble |
| receptor (AIR-3) | | UCUCUUAUAGGGGAGGCUGUGG | | interleukin-6 |
| | | UGAGGGAAUAUUAAGAGAAUUAA | | receptor (sIL- |
| | | CGGUCUAGUUCACCUCGACUUC | | 6R) |
| | | UGGAGUUGACGUUGCUU | | |
| Salmonella | Polypeptide | UAGUGUGAGAGCCGUGAGUGAA | 49 | S. Typhimurium |
| Typhimurium | | AGGCCGCGACAAAGAUCGGA | | OmpC Protein |
| OmpC Protein | | | | |
| (T-2) | | | | |
| TAFapt1-S | Polypeptide | GGGUUUUCGGGAUACGCGGAGG | 50 | AML1-MTG8 |
| | | GUGGGCAAUAACCCC | | |
| Tr-8 | Polypeptide | GGGAGGACGAUGCGGGCCGUUG | 51 | CD25 |
| | | UUGUGUGCCGCCCCAGACGACU | | |
| | | CGCCCGA | | |
| Tr-1 | Polypeptide | GGGAGGACGAUGCGGUCCUGUC | 52 | IL2-CD25 |
| | | GUCUGUUCGUCCCCAGACGACU | | Complex |
| | | CGCCCGA | | |
| Tr-7 | Polypeptide | GGGAGGACGAUGCGGUGAGUCG | 53 | IL2-CD25 |
| | | UUCCCUUCGUCCCCAGACGACU | | Complex |
| | | CGCCCGA | | |
| SQ-2 | Polypeptide | AUACCAGCUUAUUCAAUUGCCUG | 54 | ALPPL-2 |
| | | AAAAGCUAUCGCCCAAUUCGCAG | | |
| | | UGAUAUCCUUUAAGAUAGUAAGU | | |
| | | GCAAUCU | | |
| hIL-10RA Atamer | Polypeptide | CCCAUCCCUCUUCCUCUCUCCC | 55 | hIL-10RA |
| | | UCGGUACUGCUACAGCAAUGCA | | |
| | | UCUACGUCUCUGUGGAUUCGAC | | |
| | | GACUCGCUGAGAUCGAGA | | |
| RNA aptamer-1- | Polypeptide | GGGAGAGCGGAAGCGUGCUGGG | 56 | SARS-CoV |
| based sensitive | | CCUGUCGUUCGCUGUGUCUUGC | | Nucleocapsid |
| detection of | | UACGUUACGUUACACGGUUGGC | | (N) Protein |
| SARS-CoV | | AUAACCCAGAGGUCGAUGG | | |
| nucleocapsid | | | | |
| protein | | | | |
| SECURA-3 | Polypeptide | GGAGCUCAGCCUUCACUGCGAU | 57 | Pituitary tumor |
| | | CACCGUGUCGGUGGCUAGUCGA | | transforming |
| | | UGUAUCCCGAUCACCAUUGGGG | | gene 1 (PTTG1) |
| | | GCAAGUUUAGACAGGCACCACG | | |
| | | GUCGGAUCCAC | | |
| CD133-A15 | Polypeptide | CCCUCCUACAUAGGG | 58 | AC133 epitope |
| | | | | (CD133) |
| CD133-B19 | Polypeptide | CAGAACGUAUACUAUUCUG | 59 | CD133 |
| HA Clone B | Polypeptide | GGGAGAAUUCCGACCAGAAGGG | 60 | Hemagglutinin |
| | | UUAGCGGUCGUCUUAAGUAGUU | | of H3N2 |
| | | UUUGGUCCUUUCCUCUCUCCUU | | |
| | | CCUCUUCU | | |
| Anti HA Influenza | Polypeptide | GGGAGAAUUCCGACCAGAAGGG | 61 | human influenza |
| Virus Clone B | | UUAGCGGUCGUCUUAAGGUAGU | | virus |
| | | UUUUGGUCCUUUCCUCUCUCCU | | hemagglutinin |
| | | UCCUCUUCU | | |
| E. Coli Release | Polypeptide | GGACCGAGAAGUUACCCUGUAA | 62 | E. Coli Release |
| Factor 1 (Clone | | UCUUAGGAUGAAUCGCAUGCUC | | Factor 1 |
| II-1) | | UAGCGACCUUUUCGGCUUCGGC | | |
| | | GUACGCACAUCGCAGCAAC | | |
| BoxB (WT) | Polypeptide | GCCCUGAAGAAGGGC | 63 | Lambda Peptide |
| BoxB (optimized) | Polypeptide | GCCCUGAAAAAGGGC | 64 | Lambda Peptide |
| MS2 (WT) | Polypeptide | AAACAUGAGGAUAACCCAUGUCG | 65 | MS2 coat |
| | | | | protein |
| MS2 (optimized | Polypeptide | AAACAUGAGGAUCACCCAUGUCG | 66 | MS2 coat |
| | | | | protein |

TABLE 2
Small Molecule Binding Aptamers

| | | | SEQ | |
|---|---|---|---|---|
| | | | ID | |
| Aptamer | Type | Sequence (5′→3′) | NO: | Reagent |
| Cyclic Guanosine | Small | GGAUAAUAGCCGUAGGUUGCGAAA | 67 | CGMP |
| Monophosphate | Molecule | GCGACCCUGAUGAGCCCUGCGAU | | |
| (AR2) | | GCAGAAAGGUGCUGACGACACAUC | | |
| | | GAAACGGUAGCGAGAGCUC | | |
| L-tryptophan | Small | GGGAUCCUAAGCUCUAUCGGCUG | 68 | L-tryptophan |
| (Trp70-727) | Molecule | GACGACGGGGACGCCACUGGACU | | |
| | | AGGUAAGCCAGGACCGUACGUCG | | |
| | | GGAGCCGUCAGAAUAAAAGCGGCC | | |
| | | UAGCGAUCGAU | | |
| Caffeine | Small | GGAUGUCCAGUCGCUUGCAAUGC | 69 | Caffeine |
| (S2.caf.D11) | Molecule | CCUUUUAGACCCUGAUGAGGAUCA | | |
| | | UCGGACUUUGUCCUGUGGAGUAA | | |
| | | GAUCGCGAAACGGUGAAAGCCGUA | | |
| | | GGUCU | | |
| Aspartame | Small | GGAUGUCCAGUCGCUUGCAAUGC | 70 | Aspartame |
| | Molecule | CCUUUUAGACCCUGAUGAGCGGU | | |
| | | GCUAGUUAGUUGCAGUUUCGGUU | | |
| | | GUUACGCGAAACGGUGAAAGCCG | | |
| | | UAGGUCU | | |
| Theophylline + | Small | GGAUAAUAGCCGUAGGUUGCGAAA | 71 | Theophylline |
| Flavin | Molecule | GCGACCCUGAUGAGCCUUAGGAU | | and FMN |
| Mononucleotide | | AUGUCUGGAUACCAUGCAUGAUGC | | |
| (AR7) | | ACCUUGGCAGUCUUACAGAAGGAC | | |
| | | GUCGAAACGGUAGCGAGAGCUC | | |
| Kanamycin A (sla | Small | GGGAAUGGAUCCACAUCUACGAAU | 72 | Kanamycin A |
| 110) | Molecule | UCGAAUUGCCGUAAUUUCCCGUG | | |
| | | GAGCGAUGCUUCACUGCAGACUU | | |
| | | GACGAAGCUU | | |
| Malachite Green | Small | GGAUCCCGACUGGCGAGAGCCAG | 73 | Malchite Green |
| (MG-4) | Molecule | GUAACGAAUGGAUCC | | |
| ATP (ATP-40-1) | Small | GGGUUGGGAAGAAACUGUGGCAC | 74 | ATP |
| | Molecule | UUCGGUGCCAGCAACCC | | |
| Methylenedianiline | Small | GGGAGACAAGAAUAAACGCUCAAC | 75 | Methylenedianiline |
| (M1) | Molecule | UGCGAUCAGGGGUAAAUUUCCGC | | |
| | | GCAGGCUCCACGCCGCUUCGACA | | |
| | | GGAGGCUCACAACAGGC | | |
| Adenine (12E4) | Small | GGGAGAGGAUACUACACGUGAUA | 76 | Adenine |
| | Molecule | GGACGAUUAUCGAAAAUCACCAGA | | |
| | | UUGGACCCUGGUUAACGAUCCAUU | | |
| | | GCAUGUAGCAGAAGCUUCCG | | |
| L-Arginine | Small | GGAGCUCAGCCUUCACUGCAUGA | 77 | L-Arginine |
| (ag.06) | Molecule | UAAACCGAUGCUGGGCGAUUCUC | | |
| | | CUGAAGUAGGGGAAGAGUUGUCA | | |
| | | UGUAUGGGGGCACCACGGUCGGA | | |
| | | UCCUG | | |
| Dopamine | Small | GGGAAUUCCGCGUGUGCGCCGCG | 78 | Dopamine |
| (dopa2/c.1) | Molecule | GAAGACGUUGGAAGGAUAGAUACC | | |
| | | UACAACGGGGAAUAUAGAGGCCAG | | |
| | | CACAUAGUGAGGCCCUCCUCCC | | |
| RNA Tobramycin | Small | GGAAUGGAUCCACAUCUACGAAGG | 79 | Tobramycin |
| Molecular | Molecule | CUUUGAAGGUGAGACCGUGCAAAU | | |
| Beacon (BA 14- | | GAGGAUGGUGUGGAUGAUUAGGG | | |
| 2) | | UUGUCGGUUUUCACUGCAGACUU | | |
| | | GACGAAGCUU | | |
| Tobramycin (J6) | Small | GGGAGAAUUCCGACCAGAAGCUUA | 80 | Tobramycin |
| | Molecule | GUAUAGCGAGGUUUAGCUACACUC | | |
| | | GUGCUGAUCGUUUGGUACGGGAC | | |
| | | CUGCGUGUAGCCCAUAUGUGCGU | | |
| | | CUACAUGGAUCCUCA | | |
| L-Isoleucine (IL | Small | GGUCUUACGUCGUUCGCGACUAU | 81 | L-Isoleucine |
| 42-32b) | Molecule | UGGGAGACC | | |
| CAMP | Small | GGAAGAGAUGGCGACUAAAACGAC | 82 | CAMP |
| | Molecule | UUGUCGC | | |
| Xanthine (XAB) | Small | GGCACGUGUAUUACCCUAGUGGU | 83 | Xanthine |
| | Molecule | CGACGUGCC | | |
| Cyanocobalamin | Small | CCGGUGCGCAUAACCACCUCAGU | 84 | Cyanocobalamin |
| (35-mer) | Molecule | GCGAGCAAGGAA | | |
| L-Histidine (His | Small | GGCAUCGGAAAGUGGGUUGAUGU | 85 | L-Histidine |
| 945) | Molecule | AAGUAACAGGCGAUGCC | | |
| S-adenosyl | Small | GGGCGGAUGAGACGCUUGGCGUG | 86 | S-adenosyl |
| homocysteine | Molecule | UGCUGUGGAGAGUCAUCCG | | homocysteine |
| (CTH-5) | | | | |
| L-Citrulline | Small | GACGAGAAGGAGUGCUGGUUAUA | 87 | L-Citrulline |
| (44Cit11) | Molecule | CUAGCGGUUAGGUCACUCGUC | | |
| Adenosine | Small | GGGAGAUCUACGGAUCUCAGGGC | 88 | Adenosine |
| Triphosphate (1- | Molecule | UCUUACGGGAGCUACAUGGAAGG | | Triphosphate/ |
| 1 MIN) | | AGUCCAUGUGU | | ATP |
| D-Tryptophan | Small | AGUAAUACGACUCACUAUAGGGAG | 89 | D-Tryptophan |
| (MF-10) | Molecule | AAUUCCGACCAGAAGUUGGCGUU | | |
| | | GGCAUGACGCGGGGAAUCGGGUG | | |
| | | CAUCGAUGACUACUCCUGGGCCCA | | |
| | | CGUCUGUUGUUGACGUCACAGCU | | |
| | | UGAUUUAGGAUAGCGCUUGGGCA | | |
| | | GUCGUGCAGUGGA | | |
| Theophylline | Small | AGUGAUACCAGCAUCGUCUUGAUG | 90 | Theophylline |
| (mTCT8-4) | Molecule | CCCUUGGCAGCACU | | |
| NAD | Small | GGAACCCAACUAGGCGUUUGAGG | 91 | NAD |
| | Molecule | GGAUUCGGCCACGGUAACAACCCC | | |
| | | UC | | |
| Chloramphenicol | Small | GGGAUCACAGUGAAAAAAGACGUG | 92 | Chloramphenicol |
| (Cm1) | Molecule | UGAAUGUCACACUGAAAAAAGAUC | | |
| | | CC | | |
| Tetracycline | Small | GGCCUAAAACAUACCAGAUUUCGA | 93 | Tetracycline |
| (cb28 minimer) | Molecule | UCUGGAGAGGUGAAGAAUUCGAC | | |
| | | CACCUAGGCCGGU | | |
| Kanamycin A (sla | Small | GGGAAUGGAUCCACAUCUACGAAU | 94 | Kanamycin A |
| 16) | Molecule | UCACCGCGGGGUUGCGGACCGGG | | |
| | | AGCUCCAGCUUCACUGCAGACUUG | | |
| | | ACGAAGCUU | | |
| L-Valine | Small | GGGAGCUCAGAAUAAACGCUCAAA | 95 | L-Valine |
| | Molecule | UCCGUGGACAGGGCGUAAGCGCC | | |
| | | UUCGACAUGAGACACGGAUCCUGC | | |
| | | GACGAAUUCAGC | | |
| Biotin | Small | GGAACACUAUCCGAUGGCACCGAC | 96 | Biotin |
| | Molecule | CAUAGGCUCGGGUUGCCAGAGGU | | |
| | | UCCACACUUUCAUCGAAAAGCCUA | | |
| | | UGCUAGGCAAUGACAUGGACUCCU | | |
| | | UGGUCAUUAGGAUCG | | |
| Kanamycin B | Small | GGGAGCUCGGUACCGAAUUCUCG | 97 | Kanamycin B |
| (K8 | Molecule | CCCUAUAGGGGUGUUGAGGGAAA | | |
| | | UGUGUGCGACAAGGUGCGGUGGC | | |
| | | CAGAACUUUUCGUUCUCAUCAAAA | | |
| | | GCUUUGCAGAGGAUCCUU | | |
| L-Arginine | Small | GACGAGAAGGAGCGCUGGUUCUA | 98 | L-Arginine |
| (44Arg11) | Molecule | CUAGCAGGUAGGUCACUCGUC | | |
| Thyroxine (AT4-A′) | Small | GGUGGAGGGGGACGUGCUGCAUC | 99 | Thyroxine |
| | Molecule | CGCAGUGCGUCUUGGGUUGUG | | |
| Heteroaryl | Small | GGGUAGGCCAGGCAGCCAACUAG | 100 | Heteroaryldihydr |
| dihydropyrimidine | Molecule | CGAGAGCUUAAAUCUCUGAGCCCG | | opyrimidine |
| (Atamer 21) | | AGAGGGUUCAGUGCUGCUUAUGU | | (HAP) |
| | | GGACGGCUU | | |
| Phenylalanine | Small | AUUGGAUCGGUAGUAUUUAGGGU | 101 | Phenylalanine |
| | Molecule | GAGACACUUCAUGCCUUUGUUGCA | | |
| | | GGCUGGGGUGAAGGCGCUACAUG | | |
| | | GCGUCUGAAA | | |
| Flavin | Small | GGAUAAUAGCCGUAGGUUGCGAAA | 102 | FMN |
| Mononucleotide | Molecule | GCGACCCUGAUGAGCCUUAGGAU | | |
| (AR5) | | AUGCAUGAUGCAGAAGGACGUCGA | | |
| | | AACGGUAGCGAGAGCUC | | |
| Cyclic Cytidine | Small | GGAUAAUAGCCGUAGGUUGCGAAA | 103 | cCMP |
| Monophosphate | Molecule | GCGACCCUGAUGAGCCUUUAGGG | | |
| (AR3) | | CCAAGUGUGGUGAAAGACACACUC | | |
| | | GAAACGGUAGCGAGAGCUC | | |
| Cyclic Adenosine | Small | GGAUAAUAGCCGUAGGUUGCGAAA | 104 | CAMP |
| Monophosphate | Molecule | GCGACCCUGAUGAGCCUGUGGAA | | |
| (AR4) | | ACAGACGUGGCACAUGACUACGUC | | |
| | | GAAACGGUAGCGAGAGCUC | | |
| Theophylline | Small | GGAUAAUAGCCGUAGGUUGCGAAA | 105 | Theophylline |
| (AR6) | Molecule | GCGACCCUGAUGAGUCUGGAUAC | | |
| | | CAUGCAUGAUGCACCUUGGCAGU | | |
| | | CUUACGAAACGGUAGCGAGAGCUC | | |
| NADH | Small | GGAACCCAACUAGGCGUUUGAGG | 106 | NADH |
| | Molecule | GGAUUCGGCCACGGUAACAACCCC | | |
| | | UC | | |
| FMN-induced | Small | GGGCGACCCUGAUGAGCCUUAGG | 107 | flavin |
| molecular switch | Molecule | AUAUGCUUCGGCAGAAGGACGUC | | mononucleotide |
| | | GAAACGGUGAAAGCCGUAGGUUG | | (FMN) |
| | | CCC | | |
| Theophylline- | Small | GGGCGACCCUGAUGAGCCUUAUA | 108 | theophylline |
| induced | Molecule | CCAGCCGAAAGGCCCUUGGCAGA | | |
| molecular switch | | CGUCGAAACGGUGAAAGCCGUAG | | |
| | | GUUGCCC | | |
| ATP-induced | Small | GGGCGACCCUGAUGAGCCUUGGG | 109 | ATP |
| molecular switch | Molecule | AAGAAACUGUGGCACUUCGGUGC | | |
| | | CAGCACGUCGAAACGGUGAAAGC | | |
| | | CGUAGGUUGCCC | | |
| ADP | Small | GGGCGACCCUGAUGAGCACACGA | 110 | ADP |
| | Molecule | GGGGGAAACCCCGGACAAUCAGAC | | |
| | | ACGGUGUUCGAAACGGUGAAAGC | | |
| | | CGUAGGUUGCCCUUU | | |
| Mango III A10U | Small | GGCACGUACGAAGGAAGGUUUGG | 111 | Thiazole Orange- |
| | Molecule | UAUGUGGUAUAUUCGUACGUGC | | Biotin (TO1- |
| | | | | Biotin) |
| Dopamine | Small | GUCUCUGUGUGCGCCAGAGAACA | 112 | Dopamine |
| (dopa1.30/c.30) | Molecule | CUGGGGCAGAUAUGGGCCAGCAC | | |
| | | AGAAUGAGGCCC | | |
| FMN-inhibited | Small | GGGCGACCCUGAUGAGAUGAGGA | 113 | flavin |
| molecular switch | Molecule | UAUGCUUCGGCAGAAGGCUCUCG | | mononucleotide |
| | | AAACGGUGAAAGCCGUAGGUUGC | | (FMN) |
| | | CC | | |
| Dopamine | Small | GGGAAUUCCGCGUGUGCGCCGCG | 114 | Dopamine |
| (dopa2/c.4) | Molecule | GAAGAGGGAAUAUAGAGGCCAGCA | | |
| | | CAUAGUGAGGCCCUCCUCCC | | |
| theophylline- | Small | GGGCGACCCUGAUGAGAUGAUAC | 115 | theophylline |
| inhibited | Molecule | CAGCCGAAAGGCCCUUGGCAGCU | | |
| molecular switch | | CUCGAAACGGUGAAAGCCGUAGG | | |
| | | UUGCCC | | |
| Terminal 8- | Small | GGGCGAAUUCCCGAGGACCAAAUA | 116 | DNA Lesion 7,8- |
| oxodG DNA | Molecule | GUACCACCCGGGAAAACAGCUAAU | | dihydro-8- |
| Lesion (R10-B35) | | GCCGAAACGGAGAUUUUUUCUGCA | | hydroxy-2′- |
| | | GAAGCU | | deoxyguanosine |
| | | | | (8-oxodG) |
| ATP-inhibited | Small | GGGCGACCCUGAUGAGAUGGGGA | 117 | ATP |
| molecular switch | Molecule | AGAAACUGUGGCACUUCGGUGCC | | |
| | | AGCCUCUCGAAACGGUGAAAGCCG | | |
| | | UAGGUUGCCC | | |
| Spinach | Small | GGGCUAUUGCUGGAGGGGCGCCA | 118 | 3,5-dimethoxy- |
| | Molecule | CAUGAAAGUGGUGGUUGGGUGCG | | 4- |
| | | GUCGGCGAUAGCUC | | hydroxybenzylidene |
| | | | | imidazolinone |
| | | | | (DHMBI) |
| Tetracycline | Small | GGCCUAAAACAUACCAGAUUUCGA | 119 | Tetracycline |
| | Molecule | UCUGGAGAGGUGAAGAAUUCGAC | | |
| | | CACCUAGGCCGGU | | |

TABLE 3
RNA Binding Aptamers

| | | | SEQ | |
|---|---|---|---|---|
| | | Sequence | ID | |
| Aptamer | Type | (5′→3′) | NO: | Reagent |
| Yeast | RNA | GGGAA | 120 | Yeast |
| phenylalanine | | UUCCG | | phenylalanine |
| tRNA (B2) | | CGUGU | | tRNA |
| | | GCUAC | | |
| | | GUAUC | | |
| | | UUCAG | | |
| | | GCGGU | | |
| | | AACUA | | |
| | | ACUGU | | |
| | | GCUGA | | |
| | | GUCUA | | |
| | | AUCUU | | |
| | | UGUGA | | |
| | | GGGAC | | |
| | | GGUAA | | |
| | | CAUAU | | |
| | | GGUUC | | |
| | | CCGCG | | |
| | | UGGUC | | |
| | | CGUUC | | |
| | | GGGAU | | |
| | | CCUC | | |
| E. Coli | RNA | GGUGA | 121 | E. Coli |
| 5S RNA | | UACCG | | 5S RNA |
| (Helix | | CCCAA | | |
| 89 RNA) | | GAGUU | | |
| | | CAUAU | | |
| | | CGACG | | |
| | | GCGGU | | |
| | | GU | | |

TABLE 4
Metal Binding Aptamers

| | | | SEQ ID | |
|---|---|---|---|---|
| Aptamer | Type | Sequence (5′→3′) | NO: | Reagent |
| Nickel (N1) | Metal | GGGAGAGGAUACUACACGUGGAAA | 122 | Nickel |
| | | AACCAACAAAUUGGGAAAAAUGUU | | |
| | | AAGGGUCCACUUCAUGCCAUUGCA | | |
| | | UGUAGCAGAAGCUUCCG | | |
| Co²⁺ (AR1) | Metal | GGAUAAUAGCCGUAGGUUGCGAAA | 123 | Co²⁺ |
| | | GCGACCCUGAUGAGAAAGCCAAAG | | |
| | | CCGUAGCGCAGAUGAUCUCGCCA | | |
| | | UCAGUACCGAAACGGUAGCGAGAG | | |
| | | CUC | | |
| Cd²⁺ (AR1) | Metal | GGAUAAUAGCCGUAGGUUGCGAAA | 123 | Cd²⁺ |
| | | GCGACCCUGAUGAGAAAGCCAAAG | | |
| | | CCGUAGCGCAGAUGAUCUCGCCA | | |
| | | UCAGUACCGAAACGGUAGCGAGAG | | |
| | | CUC | | |
| Ni²⁺ (AR1) | Metal | GGAUAAUAGCCGUAGGUUGCGAAA | 123 | Ni²⁺ |
| | | GCGACCCUGAUGAGAAAGCCAAAG | | |
| | | CCGUAGCGCAGAUGAUCUCGCCA | | |
| | | UCAGUACCGAAACGGUAGCGAGAG | | |
| | | CUC | | |
| Zn²⁺ (AR1) | Metal | GGAUAAUAGCCGUAGGUUGCGAAA | 123 | Zn²⁺ |
| | | GCGACCCUGAUGAGAAAGCCAAAG | | |
| | | CCGUAGCGCAGAUGAUCUCGCCA | | |
| | | UCAGUACCGAAACGGUAGCGAGAG | | |
| | | CUC | | |
| Mn²⁺ (AR1) | Metal | GGAUAAUAGCCGUAGGUUGCGAAA | 123 | Mn²⁺ |
| | | GCGACCCUGAUGAGAAAGCCAAAG | | |
| | | CCGUAGCGCAGAUGAUCUCGCCA | | |
| | | UCAGUACCGAAACGGUAGCGAGAG | | |
| | | CUC | | |
| Zinc | Metal | GGGAGAGGAUACUACUGUCAUAC | 124 | Zinc |
| | | GUUAGGCUGUAGGCGAGGUGAAA | | |
| | | UGAGCGGUAAUAGCCUCAGCGUA | | |
| | | GCAUAUGCAUGAAUUCGAAGCUUC | | |
| | | GC | | |

One of skill in the art would recognize that many aptamer sequences and their cognate reagents are well known in the art and could be accessed via any suitable database. For example, a number of aptamer sequences and their cognate reagents can be found in the Aptagen database (aptagen.com) or in the Registry of Standard Biological Parts (parts.igem.org/DNA/Aptamer). Other databases are well known to the skilled artisan. The aptamer sequences listed in each of the foregoing and other known databases as well as their cognate reagents are herein incorporated by reference in their entirety.

The methods described herein may include attaching an aptamer to a polyribonucleotide. One of skill in the art would understand that a portion of the aptamer may be present on the polyribonucleotide and the method may include attaching a second portion of the aptamer to the polyribonucleotide, thus forming a complete aptamer.

### Particles

The reagents described herein may be conjugated (e.g., directly or indirectly) to a particle, e.g., a magnetic particle or a bead. In some embodiments, the reagent is conjugated to a plurality of particles. In some embodiments, a particle is conjugated to a plurality of reagents.

Magnetic particles include at least one component that is responsive to a magnetic force. A magnetic particle may be entirely magnetic or may contain components that are non-magnetic. A magnetic particle may be a magnetic bead, e.g., a substantially spherical magnetic bead. The magnetic particle may be entirely magnetic or may contain one or more magnetic cores surrounded by one or more additional materials, such as, for example, one or more functional groups and/or modifications for binding one or more target molecules. In some examples, a magnetic particle may contain a magnetic component and a surface modified with one or more silanol groups. Magnetic particles of this type may be used for binding target nucleic acid molecules.

A particle, e.g., a magnetic particle or a bead, may be porous, non-porous, hollow, solid, semi-solid, semi-fluidic, fluidic, and/or a combination thereof. In some instances, a particle, e.g., a bead, may be dissolvable or degradable. In some cases, a particle, e.g., a bead, may not be degradable. In some embodiments, the bead is composed of crosslinked agarose, e.g., SEPHAROSE®.

A particle, e.g., a magnetic particle or a bead, may include natural and/or synthetic materials. For example, a particle, e.g., a bead, can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead.

Particles, e.g., beads or magnetic particles, may be of uniform size or heterogeneous size. In some cases, the diameter of a particle, e.g., a bead, may be at least about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, 2 mm, 3 mm, 4 mm, 5 mm, 6 mm, 7 mm, 8 mm, 9 mm, 10 mm, or greater. In some cases, a particle, e.g., a bead, may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, 2 mm, 3 mm, 4 mm, 5 mm, 6 mm, 7 mm, 8 mm, 9 mm, 10 mm or less. In some cases, a particle, e.g., a bead, may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm, 500 μm-1 mm, 1 mm-2 mm, 1-5 mm, or 1-10 mm.

Particles may be of any suitable shape. Examples of particles, e.g., magnetic particles or beads, shapes include, but are not limited to, spherical, non-spherical, oval, oblong, amorphous, circular, cylindrical, and variations thereof.

### Linkers

In some embodiments, a linker is used to conjugate two or more components used in a composition or method described herein. For example, a linker may be used to conjugate a reagent to a particle (e.g., a bead), an aptamer to a linear polyribonucleotide or any combination or variation thereof. In some embodiments, the aptamer is conjugated to the linear polyribonucleotide with a chemical linker. In some embodiments, the reagent is conjugated to the particle with a chemical linker. The particle may be, for example, a magnetic particle or a bead. The bead may be, e.g., a crosslinked agarose, e.g., a SEPHAROSE®, bead. In some embodiments, a reagent is conjugated directly to a particle (e.g., a bead, e.g., a magnetic bead or crosslinked agarose, e.g., SEPHAROSE® bead).

A chemical linker provides space, rigidity, and/or flexibility between, for example, a reagent and a particle or an aptamer and a linear polyribonucleotide. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

In some embodiments, a linker may include a synthetic group derived from, e.g., a synthetic polymer (e.g., a polyethylene glycol (PEG) polymer). The chemical linker may include, e.g., triethylene glycol (TEG). In some embodiments, a linker may include one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may include one or more optionally substituted C₁-C₂₀ alkylene, optionally substituted C₁-C₂₀ heteroalkylene (e.g., a PEG unit), optionally substituted C₂-C₂₀ alkenylene (e.g., C₂ alkenylene), optionally substituted C₂-C₂₀ heteroalkenylene, optionally substituted C₂-C₂₀ alkynylene, optionally substituted C₂-C₂₀ heteroalkynylene, optionally substituted C₃-C₂₀cycloalkylene (e.g., cyclopropylene, cyclobutylene), optionally substituted C₂-C₂₀ heterocycloalkylene, optionally substituted C₄-C₂₀ cycloalkenylene, optionally substituted C₄-C₂₀ heterocycloalkenylene, optionally substituted C₈-C₂₀ cycloalkynylene, optionally substituted C₁-C₂₀ heterocycloalkynylene, optionally substituted C₅-C₁₅ arylene (e.g., C₆ arylene), optionally substituted C₃-C₁₅ heteroarylene (e.g., imidazole, pyridine), O, S, NRi (Ri is H, optionally substituted C₁-C₂₀ alkyl, optionally substituted C₁-C₂₀ heteroalkyl, optionally substituted C₂-C₂₀ alkenyl, optionally substituted C₂-C₂₀ heteroalkenyl, optionally substituted C₂-C₂₀ alkynyl, optionally substituted C₂-C₂₀ heteroalkynyl, optionally substituted C₃-C₂₀ cycloalkyl, optionally substituted C₂-C₂₀ heterocycloalkyl, optionally substituted C₄-C₂₀ cycloalkenyl, optionally substituted C₄-C₂₀ heterocycloalkenyl, optionally substituted C₈-C₂₀ cycloalkynyl, optionally substituted C₈-C₂₀ heterocycloalkynyl, optionally substituted C₅-C₁₅ aryl, or optionally substituted C₃-C₁₅ heteroaryl), P, carbonyl, thiocarbonyl, sulfonyl, phosphate, phosphoryl, or imino.

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide may accomplished using techniques known in the art.

### Resins

In some embodiments, the methods described herein include using a resin with a plurality of particles conjugated to a reagent that binds to the aptamer. The methods may include using a column that includes the resin. The method may include collecting an eluate (e.g., not bound to the resin) that includes a portion of the sample that is not bound to the reagent from the plurality of polyribonucleotides in the sample. In some embodiments, the resin includes cross-linked poly[styrene-divinylbenzene], agarose, or SEPHAROSE®.

Compositions and methods of the invention can use a surface linked to the reagent that is configured to bind to an aptamer. The surface of the resin refers to a part of a support structure (e.g., a substrate) that is accessible to contact with one or more reagents. The shape, form, materials, and modifications of the surface of the resin can be selected from a range of options depending on the application. In one embodiment, the surface of the resin is SEPHAROSE®. In one embodiment, the surface of the resin is agarose

The surface of the resin can be substantially flat or planar. Alternatively, the surface of the resin can be rounded or contoured. Exemplary contours that can be included on a surface of the resin are wells, depressions, pillars, ridges, channels or the like.

Exemplary materials that can be used as a surface of the resin include, but are not limited to acrylics, carbon (e.g., graphite, carbon-fiber), cellulose (e.g., cellulose acetate), ceramics, controlled-pore glass, cross-linked polysaccharides (e.g., agarose or SEPHAROSE®), gels, glass (e.g., modified or functionalized glass), gold (e.g., atomically smooth Au(I 11)), graphite, inorganic glasses, inorganic polymers, latex, metal oxides (e.g., Si02, Ti02, stainless steel), metalloids, metals (e.g., atomically smooth Au(I 11)), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™. A single material or mixture of several varied materials can form a resin useful in the invention.

In some embodiments, a surface of the resin includes a polymer.

In some embodiments, a surface of the resin includes SEPHAROSE®. An example is shown below, where n is any positive integer:

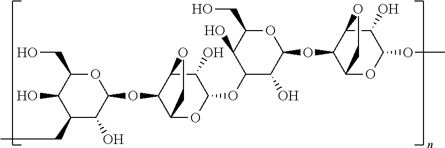

In some embodiments, a surface of the resin includes agarose. An example is shown below, where n is a positive integer:

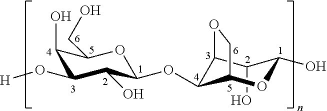

Structure of agarose: D-galactose and 3,6-anhydro-a-L-galactopyranose repeating Unit.

In some embodiments, a surface of the resin includes a polystyrene-based polymer. A polystyrene divinyl benzene copolymer synthesis schematic is shown below:

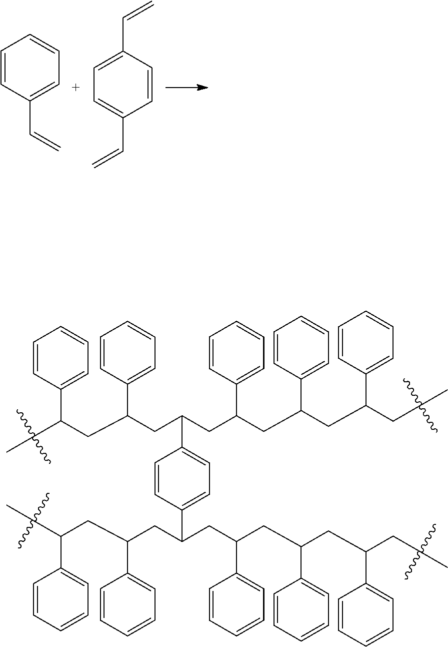

In some embodiments, a surface of the resin includes an acrylic based polymer. Poly (methyl meth acrylate) is an example shown below, wherein n is any positive integer:

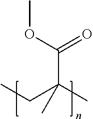

In some embodiments, a surface of the resin includes a dextran-based polymer. A Dextran example is shown below:

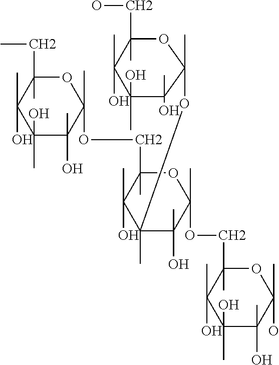

In some embodiments, a surface of the resin includes silica. An example is shown below:

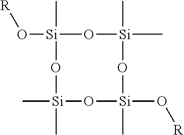

In some embodiments, a surface of the resin includes a polyacrylamide. An example cross-linked to N—N-methylenebisacrylamide is shown below:

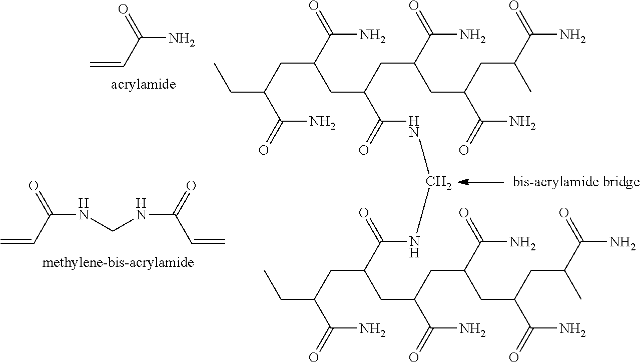

In some embodiments, a surface of the resin includes tentacle-based phases, e.g., methacrylate based.

A number of surfaces known in the art are suitable for use with the methods of the invention. Suitable surfaces may include materials including but not limited to borosilicate glass, agarose, SEPHAROSE®, magnetic beads, polystyrene, polyacrylamide, membranes, silica, semiconductor materials, silicon, organic polymers, ceramic, glass, metal, plastic polycarbonate, polycarbonate, polyethylene, polyethyleneglycol terephthalate, polymethylmethacrylate, polypropylene, polyvinylacetate, polyvinylchloride, polyvinylpyrrolidinone, and soda-lime glass.

In one embodiment, the surface of the resin is modified to contain channels, patterns, layers, or other configurations (e.g., a patterned surface). The surface can be in the form of a bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial. The surface can be a singular discrete body (e.g., a single tube, a single bead), any number of a plurality of surface bodies (e.g., a rack of 10 tubes, several beads), or combinations thereof (e.g., a tray includes a plurality of microtiter plates, a column filled with beads, a microtiter plate filed with beads).

In some embodiments, a surface can include a membrane-based resin matrix. In some embodiments, the surface of the resin includes a porous resin or a non-porous resin. Examples of porous resins can include additional agarose-based resins (e.g., cyanogen bromide activated SEPHAROSE® (GE); WorkBeads™ 40 ACT and WorkBeads 40/10000 ACT (Bioworks)), methacrylate: (Tosoh 650M derivatives etc.), polystyrene divinylbenzene (Life Tech Poros media/GE Source media), fractogel, polyacrylamide, silica, controlled pore glass, dextran derivatives, acrylamide derivatives, additional polymers, and combinations thereof.

In some embodiments, a surface can include one or more pores. In some embodiments, pore sizes can be from 300 to 8,000 Angstroms, e.g., 500 to 4,000 Angstroms in size.

A resin as described herein includes a plurality of particles. Examples of particle sizes are 5 μm-500 μm, 20 μm-300 μm, and 50 μm-200 μm. In some embodiments, particle size can be 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, or 200 μm.

A reagent can be immobilized, coated on, bound to, stuck, adhered, or attached to any of the forms of surfaces described herein (e.g., bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial).

In one embodiment, the surface is modified to contain chemically modified sites that can be used to attach (e.g., either covalently or non-covalently) the reagent to discrete sites or locations on the surface. Chemically modified sites include for example, the addition of a pattern of chemical functional groups including amino groups, carboxy groups, oxo groups and thiol groups, which can be used to covalently attach the reagent, which generally also contain corresponding reactive functional groups. Examples of surface functionalization are amino derivatives, thiol derivatives, aldehyde derivatives, formyl derivatives, azide derivatives (click chemistry), biotin derivatives, alkyne derivatives, hydroxyl derivatives, activated hydroxyls or derivatives, carboxylate derivatives, activated carboxylate derivates, activated carbonates, activated esters, NHS ester (succinimidyl), NHS carbonate (succinimidyl), Imidoester or derivated, cyanogen bromide derivatives, maleimide derivatives, haloacteyl derivatives, iodoacetamide/iodoacetyl derivatives, epoxide derivatives, streptavidin derivatives, tresyl derivatives, diene/conjugated diene derivatives (Diels-Alder type reaction), alkene derivatives, substituted phosphate derivatives, bromohydrin/halohydrin, substituted disulfides, pyridyl-disulfide derivatives, aryl azides, acyl azides, azlactone, hydrazide derivatives, halobenzene derivatives, nucleoside derivatives, branching/multi-functional linkers, dendrimeric functionalities, nucleoside derivatives, or any combination thereof.

In some embodiments, the binding capacity of the linked surface can be at least 1 mg/mL, 5 mg/mL, 10 mg/mL, 20 mg/mL, 30 mg/mL, 40 mg/mL, 50 mg/mL, or more.

In some embodiments, a column that includes the resin is configured bind at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more) of polyribonucleotides, e.g., with the aptamer. In some embodiments, the column is configured to bind from 500 μg to 1,000 mg of polyribonucleotides, e.g., with the aptamer

### Compositions

As described herein, the invention features a composition that includes a population of polyribonucleotides produced by a method as described herein. The population may include, e.g., a circular polyribonucleotide lacking an aptamer, and the circular polyribonucleotide includes at least 1% (e.g., at least 5%, e.g., at least 10%, at least 20%, at least 30%, at least 40%, at least 50%, 60%, 70%, 80%, 90%, 95%, 97%, 99%, or more) (mol/mol) of the total polyribonucleotides in the composition. In some embodiments, the population has less than 50% (e.g., less than 40%, 30%, 20%, 10%, 5%, 4%, 3%, 2%, or 1%) (mol/mol) linear polyribonucleotides in the composition.

In other embodiments, the population may include, e.g., a polyribonucleotide in a first conformation having the aptamer and the polyribonucleotide in a second conformation having the aptamer, and the polyribonucleotide in the first conformation includes at least 1%, e.g., at least 5%, e.g., at least 10%, at least 20%, at least 30%, or at least 40% (e.g., at least 50%, 60%, 70%, 80%, 90%, 95%, 97%, 99%, or more) (mol/mol) of the total polyribonucleotides in the composition.

In some embodiments as described herein, the invention features a composition that includes a mixture of polyribonucleotides. A first subset of the mixture includes a circular polyribonucleotide lacking an aptamer, and a second subset of the plurality of the polyribonucleotides includes a linear polyribonucleotide having the aptamer. The first subset includes at least 1%, e.g., at least 5%, e.g., at least 10%, at least 20%, at least 30%, or at least 40% (e.g., at least 50%, 60%, 70%, 80%, 90%, 95%, 97%, 99%, or more) (mol/mol) of the total polyribonucleotides in the composition. In some embodiments, the linear polyribonucleotides include a variety of distinct linear polyribonucleotide species, e.g., each containing the aptamer.

In some embodiments as described herein, the invention features a composition that includes a polyribonucleotide having an aptamer and a reagent configured to bind to the aptamer, wherein the reagent is conjugated to a particle, e.g., via a linker.

In some embodiments of any of the compositions as described herein, the linear polyribonucleotide includes an intron or portion thereof. The aptamer may be located 5′ or 3′ to the intron or portion thereof.

In some embodiments, the polyribonucleotide may be a modified polyribonucleotide.

In an embodiment, a circular polyribonucleotide preparation (e.g., a circular polyribonucleotide pharmaceutical preparation or composition or an intermediate in the production of the circular polyribonucleotide preparation) is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), or 100% (w/w) pure on a mass basis. Purity may be measured by any one of a number of analytical techniques known to one skilled in the art, such as, but not limited to, the use of separation technologies such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of a separation technology by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection.

In some embodiments, purity can be measured by biological test methodologies (e.g., cell-based or receptor-based tests). In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w) or 100% (w/w) of the total of mass ribonucleotide in the a preparation described herein is contained in circular polyribonucleotide molecules. The percent may be measured by any one of a number of analytical techniques known to one skilled in the art such as, but not limited to, the use of a separation technology such as chromatography (using a column, using a paper, using a gel, using HPLC, using UHPLC, etc., or by IC, by SEC, by reverse phase, by anion exchange, by mixed mode, etc.) or electrophoresis (UREA PAGE, chip-based, polyacrylamide gel, RNA, capillary, c-IEF, etc.) with or without pre- or post-separation derivatization methodologies using detection techniques based on mass spectrometry, UV-visible, fluorescence, light scattering, refractive index, or that use silver or dye stains or radioactive decay for detection. Alternatively, purity may be determined without the use of separation technologies by mass spectrometry, by microscopy, by circular dichroism (CD) spectroscopy, by UV or UV-vis spectrophotometry, by fluorometry (e.g., Qubit), by RNAse H analysis, by surface plasmon resonance (SPR), or by methods that utilize silver or dye stains or radioactive decay for detection.

In an embodiment, a circular polyribonucleotide preparation (e.g., a circular polyribonucleotide pharmaceutical preparation or composition or an intermediate in the production of the circular polyribonucleotide preparation) has a circular polyribonucleotide concentration of at least 0.1 ng/mL, 0.5 ng/mL, 1 ng/mL, 5 ng/mL, 10 ng/mL, 50 ng/mL, 0.1 μg/mL, 0.5 μg/mL, 1 μg/mL, 2 μg/mL, 5 μg/mL, 10 μg/mL, 20 μg/mL, 30 μg/mL, 40 μg/mL, 50 μg/mL, 60 μg/mL, 70 μg/mL, 80 μg/mL, 100 μg/mL, 200 μg/mL, 300 μg/mL, 500 μg/mL, 1000 μg/mL, 5000 μg/mL, 10,000 μg/mL, 100,000 μg/mL, 200 mg/mL, 300 mg/mL, 400 mg/mL, 500 mg/mL, 600 mg/mL, 650 mg/mL, 700 mg/mL, or 750 mg/mL. In an embodiment, a circular polyribonucleotide preparation (e.g., a circular polyribonucleotide pharmaceutical preparation or composition or an intermediate in the production of the circular polyribonucleotide preparation) is substantially free of mononucleotide or has a mononucleotide content of no more than 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL. In an embodiment, a circular polyribonucleotide preparation (e.g., a circular polyribonucleotide pharmaceutical preparation or composition or an intermediate in the production of the circular polyribonucleotide preparation) has a mononucleotide content from the limit of detection up to 1 μg/ml, 10 μg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) has mononucleotide content no more than 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), or any percentage therebetween of total nucleotides on a mass basis, wherein total nucleotide content is the total mass of deoxyribonucleotide molecules and ribonucleotide molecules.

In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/mL, 10 mg/mL, 50 mg/mL, 100 mg/mL, 200 mg/mL, 300 mg/mL, 400 mg/mL, 500 mg/mL, 600 mg/mL, 650 mg/mL, 700 mg/mL, 700 ng/mL, 750 mg/mL, 800 ng/mL, 850 ng/mL, 900 ng/mL, 950 ng/mL, or 1 μg/mL. In an embodiment, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, from the limit of detection of up to 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 650 mg/mL, 700 mg/mL, 700 ng/mL, 750 mg/mL, 800 ng/mL, 850 ng/mL, 900 ng/mL, 950 ng/mL, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, 2 mg/ml, 5 mg/ml, 10 mg/ml, 50 mg/ml, 100 mg/ml, 200 mg/ml, 300 mg/ml, 400 mg/ml, 500 mg/ml, 600 mg/ml, 650 mg/ml, 700 mg/ml, or 750 mg/ml.

In an embodiment, a circular polyribonucleotide preparation (e.g., a circular polyribonucleotide pharmaceutical preparation or composition or an intermediate in the production of the circular polyribonucleotide preparation) has a nicked RNA content of no more than 10% (w/w), 9.9% (w/w), 9.8% (w/w), 9.7% (w/w), 9.6% (w/w), 9.5% (w/w), 9.4% (w/w), 9.3% (w/w), 9.2% (w/w), 9.1% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a nicked RNA content that as low as zero or is substantially free of nicked RNA.

In an embodiment, a circular polyribonucleotide preparation (e.g., a circular polyribonucleotide pharmaceutical preparation or composition or an intermediate in the production of the circular polyribonucleotide preparation) has a combined linear RNA and nicked RNA content of no more than 30% (w/w), 25% (w/w), 20% (w/w), 15% (w/w), 10% (w/w), 9% (w/w), 8% (w/w), 7% (w/w), 6% (w/w), 5% (w/w), 4% (w/w), 3% (w/w), 2% (w/w), 1% (w/w), 0.5% (w/w), or 0.1% (w/w), or percentage therebetween. In an embodiment, a circular polyribonucleotide preparation (e.g., a circular polyribonucleotide pharmaceutical preparation or composition or an intermediate in the production of the circular polyribonucleotide preparation) has a combined nicked RNA and linear RNA content that is as low as zero or is substantially free of nicked and linear RNA.

In some embodiments, a circular polyribonucleotide preparation (e.g., a circular polyribonucleotide pharmaceutical preparation or composition or an intermediate in the production of the circular polyribonucleotide preparation) has a linear RNA content, e.g., linear RNA counterpart or RNA fragments, of no more than the detection limit of analytical methodologies, such as methods utilizing mass spectrometry, UV spectroscopic or fluorescence detectors, light scattering techniques, surface plasmon resonance (SPR) with or without the use of methods of separation including HPLC, by HPLC, chip or gel based electrophoresis with or without using either pre or post separation derivatization methodologies, methods of detection that use silver or dye stains or radioactive decay, or microscopy, visual methods or a spectrophotometer.

In an embodiment, a circular polyribonucleotide preparation (e.g., a circular polyribonucleotide pharmaceutical preparation or composition or an intermediate in the production of the circular polyribonucleotide preparation) has no more than 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of linear RNA.

In some embodiments, the linear polyribonucleotide molecules of the circular polyribonucleotide preparation include the linear counterpart or a fragment thereof of the circular polyribonucleotide molecule. In some embodiments, the linear polyribonucleotide molecules of the circular polyribonucleotide preparation include the linear counterpart (e.g., a pre-circularized version). In some embodiments, the linear polyribonucleotide molecules of the circular polyribonucleotide preparation include a non-counterpart or fragment thereof to the circular polyribonucleotide. In some embodiments, the linear polyribonucleotide molecules of the circular polyribonucleotide preparation include a non-counterpart to the circular polyribonucleotide. In some embodiments, the linear polyribonucleotide molecules include a combination of the counterpart of the circular polyribonucleotide and a non-counterpart or fragment thereof of the circular polyribonucleotide. In some embodiments, the linear polyribonucleotide molecules include a combination of the counterpart of the circular polyribonucleotide and a non-counterpart of the circular polyribonucleotide. In some embodiments, a linear polyribonucleotide molecule fragment is a fragment that is at least 1, 2, 3, 4, 5, 6, 7, 8, 9, 10, 20, 30, 40, 50, 60, 70, 80, 90, 100, 200, 300, 400, 500, 1000, 2000, 3000, 4000, 5000, 6000, 7000, 8000, 9000, 10000, 11000, 12000, or more nucleotides in length, or any nucleotide number therebetween.

In some embodiments, a circular polyribonucleotide preparation (e.g., a circular polyribonucleotide pharmaceutical preparation or composition or an intermediate in the production of the circular polyribonucleotide preparation) has an A260/A280 absorbance ratio from about 1.6 to about 2.3, e.g., as measured by spectrophotometer. In some embodiments, the A260/A280 absorbance ratio is about 1.4, 1.5, 1.6, 1.7, 1.8, 1.9, 2.0, 2.1, 2.2, 2.3, 2.4, 2.5, or any number therebetween. In some embodiments, a circular polyribonucleotide (e.g., a circular polyribonucleotide pharmaceutical preparation or composition or an intermediate in the production of the circular polyribonucleotide) has an A260/A280 absorbance ratio greater than about 1.8, e.g., as measured by spectrophotometer. In some embodiments, the A260/A280 absorbance ratio is about 1.6, 1.7, 1.8, 1.9, 2.0, 2.1, 2.2, 2.3, 2.4, 2.5, 2.6, 2.7, 2.8, 2.9, or greater.

In some embodiments, a circular polyribonucleotide preparation (e.g., a circular polyribonucleotide pharmaceutical preparation or composition or an intermediate in the production of the circular polyribonucleotide preparation) is substantially free of an impurity or byproduct. In various embodiments, the level of at least one impurity or byproduct in a composition including the circular polyribonucleotide is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity or byproduct. In some embodiments, the level of at least one process-related impurity or byproduct is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity or byproduct. In some embodiments, the level of at least one product-related substance is reduced by at least 30% (w/w), at least 40% (w/w), at least 50% (w/w), at least 60% (w/w), at least 70% (w/w), at least 80% (w/w), at least 90% (w/w), or at least 95% (w/w) as compared to that of the composition prior to purification or treatment to remove the impurity or byproduct. In some embodiments, a circular polyribonucleotide preparation (e.g., a circular polyribonucleotide pharmaceutical preparation or composition or an intermediate in the production of the circular polyribonucleotide preparation) is further substantially free of a process-related impurity or byproduct. In some embodiments, the process-related impurity or byproduct includes a protein (e.g., a cell protein, such as a host cell protein), a deoxyribonucleic acid (e.g., a cell deoxyribonucleic acid, such as a host cell deoxyribonucleic acid), monodeoxyribonucleotide or dideoxyribonucleotide molecules, an enzyme (e.g., a nuclease, such as an endonuclease or exonuclease, or ligase), a reagent component, a gel component, or a chromatographic material. In some embodiments, the impurity or byproduct is selected from: a buffer reagent, a ligase, a nuclease, RNase inhibitor, RNase R, deoxyribonucleotide molecules, acrylamide gel debris, and monodeoxyribonucleotide molecules. In some embodiments, the pharmaceutical preparation includes protein (e.g., cell protein, such as a host cell protein) contamination, impurities, or by-products of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng of protein contamination, impurities, or by-products per milligram (mg) of the circular polyribonucleotide molecules.

In an embodiment, a circular polyribonucleotide preparation (e.g., a circular polyribonucleotide pharmaceutical preparation or composition or an intermediate in the production of the circular polyribonucleotide preparation) is substantially free of DNA content e.g., template DNA or cell DNA (e.g., host cell DNA), has a DNA content, as low as zero, or has a DNA content of no more than 1 pg/ml, 10 pg/ml, 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 1000 μg/mL, 5000 μg/mL, 10,000 μg/mL, or 100,000 μg/mL.

In an embodiment, a circular polyribonucleotide preparation (e.g., a circular polyribonucleotide pharmaceutical preparation or composition or an intermediate in the production of the circular polyribonucleotide preparation) is substantially free of DNA content, has a DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis, wherein total nucleotide molecules is the total mass of deoxyribonucleotide content and ribonucleotide molecules. In an embodiment, a circular polyribonucleotide preparation (e.g., a circular polyribonucleotide pharmaceutical preparation or composition or an intermediate in the production of the circular polyribonucleotide preparation) is substantially free of DNA content, has DNA content as low as zero, or has DNA content no more than 0.001% (w/w), 0.01% (w/w), 0.1% (w/w), 1% (w/w), 2% (w/w), 3% (w/w), 4% (w/w), 5% (w/w), 6% (w/w), 7% (w/w), 8% (w/w), 9% (w/w), 10% (w/w), 15% (w/w), 20% (w/w), 25% (w/w), 30% (w/w), 35% (w/w), 40% (w/w), 45% (w/w), 50% (w/w) of total nucleotides on a mass basis as measured after a total DNA digestion by enzymes that digest nucleosides by quantitative liquid chromatography-mass spectrometry (LC-MS), in which the content of DNA is back calculated from a standard curve of each base (i.e., A, C, G, T) as measured by LC-MS.

In an embodiment, a circular polyribonucleotide preparation (e.g., a circular polyribonucleotide pharmaceutical preparation or composition or an intermediate in the production of the circular polyribonucleotide preparation) has a protein (e.g., cell protein (CP), e.g., enzyme, a production-related protein, e.g., carrier protein) contamination, impurities, or by-products of no more than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination, impurities, or by-products from the limit of detection of up to 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, or 500 ng/ml.

In an embodiment, a circular polyribonucleotide preparation (e.g., a circular polyribonucleotide pharmaceutical preparation or composition or an intermediate in the production of the circular polyribonucleotide preparation) has a protein (e.g., production-related protein such as a cell protein (CP), e.g., enzyme) contamination, impurities, or by-products of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide. In an embodiment, a circular polyribonucleotide (e.g., a circular polyribonucleotide pharmaceutical preparation or composition or an intermediate in the production of the circular polyribonucleotide) has a protein (e.g., production-related protein such as a cell protein (CP), e.g., enzyme) contamination, impurities, or by-products from the level of detection up to 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the circular polyribonucleotide.

In an embodiment, a circular polyribonucleotide preparation (e.g., a circular polyribonucleotide pharmaceutical preparation or composition or an intermediate in the production of the circular polyribonucleotide preparation) has low levels or is absent of endotoxins, e.g., as measured by the Limulus amebocyte lysate (LAL) test. In some embodiments, the pharmaceutical preparation or compositions or an intermediate in the production of the circular polyribonucleotides includes less than 20 EU/kg (weight), 10 EU/kg, 5 EU/kg, 1 EU/kg endotoxin, or lacks endotoxin as measured by the Limulus amebocyte lysate test. In an embodiment, a circular polyribonucleotide composition has low levels or absence of a nuclease or a ligase.

In some embodiments, a circular polyribonucleotide preparation (e.g., a circular polyribonucleotide pharmaceutical preparation or composition or an intermediate in the production of the circular polyribonucleotide preparation) includes no greater than about 50% (w/w), 45% (w/w), 40% (w/w), 35% (w/w), 30% (w/w), 25% (w/w), 20% (w/w), 19% (w/w), 18% (w/w), 17% (w/w), 16% (w/w), 15% (w/w), 14% (w/w), 13% (w/w), 12% (w/w), 11% (w/w), 10% (w/w), 9% (w/w), 8% (w/w), 7% (w/w), 6% (w/w), 5% (w/w), 4% (w/w), 3% (w/w), 2% (w/w), 1% (w/w) of at least one enzyme, e.g., polymerase, e.g., RNA polymerase.

In an embodiment, a circular polyribonucleotide preparation (e.g., a circular polyribonucleotide pharmaceutical preparation or composition or an intermediate in the production of the circular polyribonucleotide preparation) is sterile or substantially free of microorganisms, e.g., the composition or preparation supports the growth of fewer than 100 viable microorganisms as tested under aseptic conditions, the composition or preparation meets the standard of USP <71>, and/or the composition or preparation meets the standard of USP <85>. In some embodiments, the pharmaceutical preparation includes a bioburden of less than 100 CFU/100 ml, 50 CFU/100 ml, 40 CFU/100 ml, 30 CFU/100 ml, 20 CFU/100 ml, 10 CFU/100 ml, or 1 CFU/100 ml before sterilization.

In some embodiments, the circular polyribonucleotide preparation can be further purified using known techniques in the art for removing impurities or byproduct, such as column chromatography or pH/vial inactivation.

In some embodiments, a total weight of polyribonucleotides in the composition includes at least 500 μg (e.g., at least 600 μg, 700 μg, 800 μg, 900 μg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, 1,000 mg, or more). In some embodiments, the total weight of polyribonucleotides in the population of polyribonucleotides is from 500 μg to 1000 mg.

### Polynucleotides

The present invention features polyribonucleotides that are used in methods of separation and/or purification and present in compositions described herein. The polyribonucleotides described herein may be linear polyribonucleotides, circular polyribonucleotides or a combination thereof. In some embodiments, a circular polyribonucleotide is produced from a linear polyribonucleotide (e.g., by splicing compatible ends of the linear polyribonucleotide). In some embodiments, a linear polyribonucleotide is transcribed from a deoxyribonucleotide template (e.g., a vector, a linearized vector, or a cDNA). Accordingly, the invention features linear deoxyribonucleotides, circular deoxyribonucleotides, linear polyribonucleotides, and circular polyribonucleotides and compositions thereof useful in the production of polyribonucleotides.

### Linear Polyribonucleotides

The present invention features linear polyribonucleotides that may include one or more of the following: a 3′ intron fragment; a 3′ splice site; a 3′ exon; a polyribonucleotide cargo; a 5′ exon; a 5′ splice site; and a 5′ intron fragment. In some embodiments, the 3′ intron fragment corresponds to a 3′ portion of a catalytic Group I intron, for example, a catalytic Group I intron from a cyanobacterium Anabaena pre-tRNA-Leu gene, a Tetrahymena pre-rRNA, a T4 phage td gene, or a variant thereof. In some embodiments, the 5′ intron fragment corresponds to a 5′ portion of a catalytic Group I intron, for example, a catalytic Group I intron from a cyanobacterium Anabaena pre-tRNA-Leu gene, a Tetrahymena pre-rRNA, a T4 phage td gene, or a variant thereof.

The linear polyribonucleotide may include additional elements, e.g., outside of or between any of elements described above. For example, any of the above elements may be separated by a spacer sequence, as described herein. An aptamer as described herein may be present in any region of the linear polyribonucleotide as described herein.

In some embodiments, a linear polyribonucleotide includes, in the following 5′-to-3′ order: an aptamer, a first circularization elements (e.g., a first intron fragment); a polyribonucleotide cargo; and a second circularization element (e.g., a second intron fragment). In some embodiments, a linear polyribonucleotides includes, in the following 5′-to-3′ order: an aptamer, a 3′ intron fragment; a 3′ splice site; a 3′ exon; a polyribonucleotide cargo; a 5′ exon; a 5′ splice site; and a 5′ intron fragment.

In some embodiments, a linear polyribonucleotide includes, in the following 5′-to-3′ order: a first circularization elements (e.g., a first intron fragment); a polyribonucleotide cargo; a second circularization element (e.g., a second intron fragment); and an aptamer. In some embodiments, a linear polyribonucleotides includes, in the following 5′-to-3′ order: a 3′ intron fragment; a 3′ splice site; a 3′ exon; a polyribonucleotide cargo; a 5′ exon; a 5′ splice site; a 5′ intron fragment; and an aptamer.

In certain embodiments, provided herein is a method of generating a linear polyribonucleotide by performing transcription (e.g., in a cell-free system, such as in vitro transcription) using a deoxyribonucleotide (e.g., a vector, linearized vector, or cDNA) provided herein as a template (e.g., a vector, linearized vector, or cDNA provided herein with an RNA polymerase promoter positioned upstream of the region that codes for the linear polyribonucleotide).

A deoxyribonucleotide template may be transcribed to a produce a linear polyribonucleotide containing the components described herein. Upon expression, the linear polyribonucleotide may produce a splicing-compatible polyribonucleotide, which may be spliced in order to produce a circular polyribonucleotide, e.g., for subsequent use.

In some embodiments, the linear polyribonucleotide is from 50 to 20,000, e.g., 300 to 20,000 (e.g., 50, 60, 70, 80, 90, 100, 150, 200, 250, 300, 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 5,000, 6,000, 7,000, 8,000, 9,000, 10,000, 11,000, 12,000, 13,000, 14,000, 15,000, 16,000, 17,000, 18,000, 19,000, or 20,000) ribonucleotides in length. The linear polyribonucleotide may be, e.g., at least 500, at least 1,000, at least 2,000, at least 3,000, at least 4,000, or at least 5,000 ribonucleotides in length.

### Circular Polyribonucleotides

In some embodiments, the invention features a circular polyribonucleotide. In embodiments, the circular polyribonucleotide includes a splice junction joining a 5′ exon and a 3′ exon. In embodiments, the circular polyribonucleotide lacks in an intron, e.g., after splicing. In embodiments, the circular polyribonucleotide lacks an aptamer, e.g., after splicing.

In embodiments, the circular polynucleotide further includes a polyribonucleotide cargo. In embodiments, the polyribonucleotide cargo includes an expression (or coding) sequence, a non-coding sequence, or a combination of an expression (or coding) sequence and a non-coding sequence. In embodiments, the polyribonucleotide cargo includes an expression (or coding) sequence encoding a polypeptide. In embodiments, the polyribonucleotide includes at least one IRES (e.g., an IRES) operably linked to an expression sequence encoding a polypeptide. In some embodiments, the circular polyribonucleotide further includes a spacer region between the at least one IRES and the 5′ exon fragment or the 3′ exon fragment. The spacer region may be, e.g., at least 5 (e.g., at least 10, at least 15, at least 20) ribonucleotides in length ribonucleotides in length. The spacer region may be, e.g., from 5 to 500 (e.g., 10, 20, 30, 40, 50, 60, 70, 80, 90, 100, 150, 200, 250, 300, 350, 400, 450, or 500) ribonucleotides. In some embodiments, the spacer region includes a polyA sequence. In some embodiments, the spacer region includes a polyA-C, polyA-G, polyA-U, or other heterogenous or random sequence.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is of a sufficient size to accommodate at least one binding site for a ribosome. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 1400 nucleotides, at least 300 nucleotides, at least 200 nucleotides, at least 100 nucleotides may be produced.

In some embodiments, the circular polyribonucleotide includes one or more elements described herein. In some embodiments, the elements are separated from one another by a spacer sequence. In some embodiments, the elements are separated from one another by 1 ribonucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides, or any amount of nucleotides therebetween. In some embodiments, one or more elements are contiguous with one another, e.g., lacking a spacer element.

In some embodiments, the circular polyribonucleotide includes one or more repetitive elements. In some embodiments, the circular polyribonucleotide includes one or more modifications described herein. In one embodiment, the circular polyribonucleotide contains at least one nucleoside modification. In one embodiment, up to 100% of the nucleosides of the circular polyribonucleotide are modified. In one embodiment, at least one nucleoside modification is a uridine modification or an adenosine modification.

As a result of its circularization, the circular polyribonucleotide may include certain characteristics that distinguish it from a linear polyribonucleotide. For example, the circular polyribonucleotide may contain an aptamer that is accessible than a linear polyribonucleotide. In some embodiments, the circular polyribonucleotide is less susceptible to degradation by exonuclease as compared to a linear polyribonucleotide. As such, the circular polyribonucleotide is more stable than a linear polyribonucleotide, especially when incubated in the presence of an exonuclease. The increased stability of the circular polyribonucleotide compared with a linear polyribonucleotide makes circular polyribonucleotide more useful as a cell transforming reagent to produce polypeptides and can be stored more easily and for longer than a linear polyribonucleotide. The stability of the circular polyribonucleotide treated with exonuclease can be tested using methods standard in art which determine whether RNA degradation has occurred (e.g., by gel electrophoresis). Moreover, unlike a linear polyribonucleotide, the circular polyribonucleotide is less susceptible to dephosphorylation when the circular polyribonucleotide is incubated with phosphatase, such as calf intestine phosphatase.

### Polyribonucleotide Cargo

A polyribonucleotide cargo described herein includes any sequence including at least one polyribonucleotide. In some embodiments, the polyribonucleotide cargo includes an expression (or coding) sequence, a non-coding sequence, or an expression (or coding) sequence and a non-coding sequence. In some embodiments, the polyribonucleotide cargo includes an expression sequence encoding a polypeptide. In some embodiments, the polyribonucleotide cargo includes an IRES operably linked to an expression sequence encoding a polypeptide. In some embodiments, the polyribonucleotide cargo includes an expression sequence that encodes a polypeptide that has a biological effect on a subject.

A polyribonucleotide cargo may, for example, include at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the polyribonucleotides cargo includes from 1-20,000 nucleotides, 1-10,000 nucleotides, 1-5,000 nucleotides, 100-20,000 nucleotide, 100-10,000 nucleotides, 100-5,000 nucleotides, 500-20,000 nucleotides, 500-10,000 nucleotides, 500-5,000 nucleotides, 1,000-20,000 nucleotides, 1,000-10,000 nucleotides, or 1,000-5,000 nucleotides.

In embodiments, the polyribonucleotide cargo includes one or multiple expression (or coding) sequences, wherein each expression sequence encodes a polypeptide. In embodiments, the polyribonucleotide cargo includes one or multiple noncoding sequences. In embodiments, the polyribonucleotide consists entirely of non-coding sequence(s). In embodiments, the polyribonucleotide cargo includes a combination of expression and noncoding sequences.

In some embodiments, polyribonucleotides made as described herein are used as effectors in therapy or agriculture. For example, a circular polyribonucleotide made by the methods described herein (e.g., the cell-free methods described herein) may be administered to a subject (e.g., in a pharmaceutical, veterinary, or agricultural composition). In another example, a circular polyribonucleotide made by the methods described herein (e.g., the cell-free methods described herein) may be delivered to a cell.

In some embodiments, the polyribonucleotide includes any feature, or any combination of features as disclosed in PCT Publication No. WO2019/118919, which is hereby incorporated by reference in its entirety.

In some embodiments, the polyribonucleotide cargo includes an open reading frame. In some embodiments, the open reading frame is operably linked to an IRES. In embodiments, the open reading frame encodes an RNA or a polypeptide. In some embodiments, the open reading frame encodes a polypeptide and the polyribonucleotide (e.g., circular polyribonucleotide) provides increased expression (e.g., by at least 5%, 10%, 15%, 20%, 25%, 30%, 40%, 50%, 60%, 70%, 80%, 90%, 100%, or more) of the polypeptide, e.g., as compared to a linear polyribonucleotide encoding the polypeptide. In some embodiments, increased purity of the polyribonucleotide, e.g., a circular polyribonucleotide, results in increased expression (e.g., by at least 5%, 10%, 15%, 20%, 25%, 30%, 40%, 50%, 60%, 70%, 80%, 90%, 100%, or more) of the polypeptide, e.g., as compared to a population of circular and linear polyribonucleotides.

### Polypeptide Expression Sequences

In some embodiments, the polyribonucleotide described herein (e.g., the polyribonucleotide cargo of a circular polyribonucleotide) includes one or more expression (or coding) sequences, wherein each expression (coding) sequence encodes a polypeptide. In some embodiments, the circular polyribonucleotide includes two, three, four, five, six, seven, eight, nine, ten or more expression sequences.

Each encoded polypeptide may be linear or branched. The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

Polypeptides included herein may include naturally occurring polypeptides or non-naturally occurring polypeptides. In some instances, the polypeptide may be a functional fragment or variant of a reference polypeptide (e.g., an enzymatically active fragment or variant of an enzyme). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

Some examples of a polypeptide include, but are not limited to, a fluorescent tag or marker, an antigen, a therapeutic polypeptide, a plant-modifying polypeptide, or a polypeptide for agricultural applications.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, and a thrombolytic.

A polypeptide for agricultural applications may be a bacteriocin, a lysin, an antimicrobial polypeptide, an antifungal polypeptide, a nodule C-rich peptide, a bacteriocyte regulatory peptide, a peptide toxin, a pesticidal polypeptide (e.g., insecticidal polypeptide or nematocidal polypeptide), an antigen binding polypeptide (e.g., antigen binding antibody or antibody-like fragments, such as single chain antibodies, nanobodies or other Ig heavy chain or light chain containing polypeptides), an enzyme (e.g., nuclease, amylase, cellulase, peptidase, lipase, chitinase), a peptide pheromone, and a transcription factor.

In some cases, the polyribonucleotide expresses a non-human protein.

In some embodiments, the polyribonucleotide expresses an antibody, e.g., an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can include more than one expression sequence, each of which expresses a portion of an antibody, and the sum of which can constitute the antibody. In some cases, the circular polyribonucleotide includes one expression sequence coding for the heavy chain of an antibody, and another expression sequence coding for the light chain of the antibody. In some cases, when the circular polyribonucleotide is expressed in a cell or a cell-free environment, the light chain and heavy chain can be subject to appropriate modification, folding, or other post-translation modification to form a functional antibody.

In embodiments, polypeptides include multiple polypeptides, e.g., multiple copies of one polypeptide sequence, or multiple different polypeptide sequences. In embodiments, multiple polypeptides are connected by linker amino acids or spacer amino acids.

In embodiments, the polynucleotide cargo includes a sequence encoding a signal peptide. Many signal peptide sequences have been described, for example, the Tat (Twin-arginine translocation) signal sequence is typically an N-terminal peptide sequence containing a consensus SRR×FLK “twin-arginine” motif, which serves to translocate a folded protein containing such a Tat signal peptide across a lipid bilayer. See also, e.g., the Signal Peptide Database publicly available at www.]signalpeptide.de. Signal peptides are also useful for directing a protein to specific organelles; see, e.g., the experimentally determined and computationally predicted signal peptides disclosed in the Spdb signal peptide database, publicly available at proline.bic.nus.edu.sg/spdb.

In embodiments, the polynucleotide cargo includes sequence encoding a cell-penetrating peptide (CPP). Hundreds of CPP sequences have been described; see, e.g., the database of cell-penetrating peptides, CPPsite, publicly available at crdd.osdd.net/raghava/cppsite/. An example of a commonly used CPP sequence is a poly-arginine sequence, e.g., octoarginine or nonoarginine, which can be fused to the C-terminus of the CGI peptide.

In embodiments, the polynucleotide cargo includes sequence encoding a self-assembling peptide; see, e.g., Miki et al. (2021) Nature Communications, 21:3412, DOI: 10.1038/s41467-021-23794-6.

In some embodiments, the expression (or coding) sequence includes a poly-A sequence (e.g., at the 3′ end of an expression sequence). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity (e.g., the level of one or more marker of an immune or inflammatory response), half-life, and/or expression efficiency.

### Therapeutic Polypeptides

In some embodiments, a polyribonucleotide described herein (e.g., the polyribonucleotide cargo of the circular polyribonucleotide) includes at least one expression sequence encoding a therapeutic polypeptide. A therapeutic polypeptide is a polypeptide that when administered to or expressed in a subject provides some therapeutic benefit. Administration to a subject or expression in a subject of a therapeutic polypeptide may be used to treat or prevent a disease, disorder, or condition or a symptom thereof. In some embodiments, the circular polyribonucleotide encodes two, three, four, five, six, seven, eight, nine, ten or more therapeutic polypeptides.

In some embodiments, the polyribonucleotide includes an expression sequence encoding a therapeutic protein. The protein may treat the disease in the subject in need thereof. In some embodiments, the therapeutic protein can compensate for a mutated, under-expressed, or absent protein in the subject in need thereof. In some embodiments, the therapeutic protein can target, interact with, or bind to a cell, tissue, or virus in the subject in need thereof.

A therapeutic polypeptide can be a polypeptide that can be secreted from a cell, or localized to the cytoplasm, nucleus, or membrane compartment of a cell.

A therapeutic polypeptide may be a hormone, a neurotransmitter, a growth factor, an enzyme (e.g., oxidoreductase, metabolic enzyme, mitochondrial enzyme, oxygenase, dehydrogenase, ATP-independent enzyme, lysosomal enzyme, desaturase), a cytokine, a transcription factor, an antigen binding polypeptide (e.g., antigen binding antibody or antibody-like fragments, such as single chain antibodies, nanobodies or other Ig heavy chain or light chain containing polypeptides), an Fc fusion protein, an anticoagulant, a blood factor, a bone morphogenetic protein, an interferon, an interleukin, a thrombolytic, an antigen (e.g., a tumor, viral, or bacterial antigen), a nuclease (e.g., an endonuclease such as a Cas protein, e.g., Cas9), a membrane protein (e.g., a chimeric antigen receptor (CAR), a transmembrane receptor, a G-protein-coupled receptor (GPCR), a receptor tyrosine kinase (RTK), an antigen receptor, an ion channel, or a membrane transporter), a secreted protein, a gene editing protein (e.g., a CRISPR-Cas, TALEN, or zinc finger), or a gene writing protein (see, e.g., International Patent Publication No. WO2020/047124, incorporated in its entirety herein by reference).

In some embodiments, the therapeutic polypeptide is an antibody, e.g., a full-length antibody, an antibody fragment, or a portion thereof. In some embodiments, the antibody expressed by the polyribonucleotide (e.g., circular polyribonucleotide) can be of any isotype, such as IgA, IgD, IgE, IgG, IgM. In some embodiments, the polyribonucleotide expresses a portion of an antibody, such as a light chain, a heavy chain, a Fc fragment, a CDR (complementary determining region), a Fv fragment, or a Fab fragment, a further portion thereof. In some embodiments, the polyribonucleotide expresses one or more portions of an antibody. For instance, the polyribonucleotide can include more than one expression sequence, each of which expresses a portion of an antibody, and the sum of which can constitute the antibody. In some cases, the polyribonucleotide includes one expression sequence coding for the heavy chain of an antibody, and another expression sequence coding for the light chain of the antibody. When the polyribonucleotide is expressed in a cell, the light chain and heavy chain can be subject to appropriate modification, folding, or other post-translation modification to form a functional antibody.

In some embodiments, polyribonucleotides made as described herein (e.g., circular polyribonucleotides) are used as effectors in therapy or agriculture. For example, a polyribonucleotide made by the methods described herein may be administered to a subject (e.g., in a pharmaceutical, veterinary, or agricultural composition). In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusca. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

### Plant-Modifying Polypeptides

In some embodiments, the polyribonucleotide described herein (e.g., the polyribonucleotide cargo of the polyribonucleotide) includes at least one expression (or coding) sequence encoding a plant-modifying polypeptide. A plant-modifying polypeptide refers to a polypeptide that can alter the genetic properties (e.g., increase gene expression, decrease gene expression, or otherwise alter the nucleotide sequence of DNA or RNA), epigenetic properties, or physiological or biochemical properties of a plant in a manner that results in a change in the plant's physiology or phenotype, e.g., an increase or decrease in plant fitness. In some embodiments, the polyribonucleotide encodes two, three, four, five, six, seven, eight, nine, ten or more different plant-modifying polypeptides, or multiple copies of one or more plant-modifying polypeptides. A plant-modifying polypeptide may change the physiology or phenotype of, or increase the fitness of, a variety of plants, or can be one that effects such change(s) in one or more specific plants (e.g., a specific species or genera of plants).

Examples of polypeptides that can be used herein can include an enzyme (e.g., a metabolic recombinase, a helicase, an integrase, a RNAse, a DNAse, or a ubiquitination protein), a pore-forming protein, a signaling ligand, a cell penetrating peptide, a transcription factor, a receptor, an antibody, a nanobody, a gene editing protein (e.g., CRISPR-Cas endonuclease, TALEN, or zinc finger), riboprotein, a protein aptamer, or a chaperone.

### Agricultural Polypeptides

In some embodiments, the polyribonucleotide described herein (e.g., the polyribonucleotide cargo of the polyribonucleotide) includes at least one expression (or coding) sequence encoding an agricultural polypeptide. An agricultural polypeptide is a polypeptide that is suitable for an agricultural use. In embodiments, an agricultural polypeptide is applied to a plant or seed (e.g., by foliar spray, dusting, injection, or seed coating) or to the plant's environment (e.g., by soil drench or granular soil application), resulting in an alteration of the plant's physiology, phenotype, or fitness. Embodiments of an agricultural polypeptide include polypeptides that alter a level, activity, or metabolism of one or more microorganisms that are resident in or on a plant or non-human animal host, the alteration resulting in an increase in the host's fitness. In some embodiments the agricultural polypeptide is a plant polypeptide. In some embodiments, the agricultural polypeptide is an insect polypeptide. In some embodiments, the agricultural polypeptide has a biological effect when contacted with a non-human vertebrate animal, invertebrate animal, microbial, or plant cell.

In some embodiments, the polyribonucleotide encodes two, three, four, five, six, seven, eight, nine, ten or more agricultural polypeptides, or multiple copies of one or more agricultural polypeptides.

Embodiments of polypeptides useful in agricultural applications include, for example, bacteriocins, lysins, antimicrobial peptides, nodule C-rich peptides, and bacteriocyte regulatory peptides. Such polypeptides can be used to alter the level, activity, or metabolism of target microorganisms for increasing the fitness of insects, such as honeybees and silkworms. Embodiments of agriculturally useful polypeptides include peptide toxins, such as those naturally produced by entomopathogenic bacteria (e.g., Bacillus thuringiensis, Photorhabdus luminescens, Serratia entomophila, or Xenorhabdus nematophila), as is known in the art. Embodiments of agriculturally useful polypeptides include polypeptides (including small peptides such as cyclodipeptides or diketopiperazines) for controlling agriculturally important pests or pathogens, e.g., antimicrobial polypeptides or antifungal polypeptides for controlling diseases in plants, or pesticidal polypeptides (e.g., insecticidal polypeptides or nematicidal polypeptides) for controlling invertebrate pests such as insects or nematodes. Embodiments of agriculturally useful polypeptides include antibodies, nanobodies, and fragments thereof, e.g., antibody or nanobody fragments that retain at least some (e.g., at least 10%) of the specific binding activity of the intact antibody or nanobody. Embodiments of agriculturally useful polypeptides include transcription factors, e.g., plant transcription factors; see, e.g, the “AtTFDB” database listing the transcription factor families identified in the model plant Arabidopsis thaliana), publicly available at agris-knowledgebase[dot] org/AtTFDB/. Embodiments of agriculturally useful polypeptides include nucleases, for example, exonucleases or endonucleases (e.g., Cas nucleases such as Cas9 or Cas12a). Embodiments of agriculturally useful polypeptides further include cell-penetrating peptides, enzymes (e.g., amylases, cellulases, peptidases, lipases, chitinases), peptide pheromones (for example, yeast mating pheromones, invertebrate reproductive and larval signaling pheromones, see, e.g., Altstein (2004) PEPTIDES, 25:1373-76).

### Internal Ribosomal Entry Sites

In some embodiments, the polyribonucleotide described herein (e.g., the polyribonucleotide cargo of the polyribonucleotide) includes one or more internal ribosome entry site (IRES) elements. In some embodiments, the IRES is operably linked to one or more expression (or coding) sequences (e.g., each IRES is operably linked to one or more expression (or coding) sequences). In embodiments, the IRES is located between a heterologous promoter and the 5′ end of a coding sequence.

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, if present, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picornavirus, Turnip crinkle virus, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus.

In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s).

In some embodiments, a polyribonucleotide cargo includes an IRES. For example, the polyribonucleotide cargo may include a circular RNA IRES, e.g., as described in Chen et al. MOL. CELL 81(20):4300-18, 2021, which is hereby incorporated by reference in its entirety.

### Regulatory Elements

In some embodiments, the polyribonucleotide described herein (e.g., the polyribonucleotide cargo of the polyribonucleotide) includes one or more regulatory elements. In some embodiments, the polyribonucleotide includes a regulatory element, e.g., a sequence that modifies expression of an expression sequence within the polyribonucleotide.

A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase the amount or number of products expressed for multiple expression sequences attached in tandem. Hence, one regulatory element can enhance the expression of one or more expression sequences. Multiple regulatory elements are well-known to persons of ordinary skill in the art.

In some embodiments, the regulatory element is a translation modulator. A translation modulator can modulate translation of the expression sequence in the polyribonucleotide. A translation modulator can be a translation enhancer or suppressor. In some embodiments, the polyribonucleotide includes at least one translation modulator adjacent to at least one expression sequence. In some embodiments, the polyribonucleotide includes a translation modulator adjacent each expression sequence. In some embodiments, the translation modulator is present on one or both sides of each expression sequence, leading to separation of the expression products, e.g., peptide(s) and or polypeptide (s).

In some embodiments, the regulatory element is a microRNA (miRNA) or a miRNA binding site.

Further examples of regulatory elements are described, e.g., in paragraphs [0154]-[0161] of International Patent Publication No. WO2019/118919, which is hereby incorporated by reference in its entirety.

### Translation Initiation Sequences

In some embodiments, the polyribonucleotide described herein (e.g., the polyribonucleotide cargo of the polyribonucleotide) includes at least one translation initiation sequence. In some embodiments, the polyribonucleotide includes a translation initiation sequence operably linked to an expression sequence.

In some embodiments, the polyribonucleotide encodes a polypeptide and may include a translation initiation sequence, e.g., a start codon. In some embodiments, the translation initiation sequence includes a Kozak or Shine-Dalgarno sequence. In some embodiments, the polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the polyribonucleotide includes at least one translation initiation sequence adjacent to an expression sequence. In some embodiments, the translation initiation sequence provides conformational flexibility to the polyribonucleotide. In some embodiments, the translation initiation sequence is within a single stranded region of the polyribonucleotide. Further examples of translation initiation sequences are described in paragraphs [0163]-[0165] of International Patent Publication No. WO2019/118919, which is hereby incorporated by reference in its entirety.

The polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the polyribonucleotide may initiate at a codon which is not the first start codon, e.g., AUG. Translation of the polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the polyribonucleotide may begin at alternative translation initiation sequence, such as ACG. As another non-limiting example, the polyribonucleotide translation may begin at alternative translation initiation sequence, CTG/CUG. As another non-limiting example, the polyribonucleotide translation may begin at alternative translation initiation sequence, GTG/GUG. As another non-limiting example, the polyribonucleotide may begin translation at a repeat-associated non-AUG (RAN) sequence, such as an alternative translation initiation sequence that includes short stretches of repetitive RNA e.g., CGG, GGGGCC, CAG, CTG.

### Termination Elements

In some embodiments, the polyribonucleotide described herein (e.g., the polyribonucleotide cargo of the polyribonucleotide) includes least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

Further examples of termination elements are described in paragraphs [0169]-[0170] of International Patent Publication No. WO2019/118919, which is hereby incorporated by reference in its entirety.

### Untranslated Regions

In some embodiments, a circular polyribonucleotide includes untranslated regions (UTRs). UTRs of a genomic region including a gene may be transcribed but not translated. In some embodiments, a UTR is included upstream of the translation initiation sequence of an expression sequence described herein. In some embodiments, a UTR is included downstream of an expression sequence described herein. In some instances, one UTR for first expression sequence is the same as or continuous with or overlapping with another UTR for a second expression sequence.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more marker of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

Any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Stagger Elements

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide includes one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (SEQ ID NO: 126) (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid (SEQ ID NO: 127). In some embodiments, this sequence includes a non-conserved sequence of amino-acids with a strong alpha-helical propensity followed by the consensus sequence -D(V/I)EXNPGP, where x=any amino acid (SEQ ID NO: 128). Some nonlimiting examples of stagger elements includes GDVESNPGP (SEQ ID NO: 129), GDIEENPGP (SEQ ID NO: 130), VEPNPGP (SEQ ID NO: 131), IETNPGP (SEQ ID NO: 132), GDIESNPGP (SEQ ID NO: 133), GDVELNPGP (SEQ ID NO: 134), GDIETNPGP (SEQ ID NO: 135), GDVENPGP (SEQ ID NO: 136), GDVEENPGP (SEQ ID NO: 137), GDVEQNPGP (SEQ ID NO: 138), IESNPGP (SEQ ID NO: 139), GDIELNPGP (SEQ ID NO: 140), HDIETNPGP (SEQ ID NO: 141), HDVETNPGP (SEQ ID NO: 142), HDVEMNPGP (SEQ ID NO: 143), GDMESNPGP (SEQ ID NO: 144), GDVETNPGP (SEQ ID NO: 145) GDIEQNPGP (SEQ ID NO: 146), and DSEFNPGP (SEQ ID NO: 147).

In some embodiments, the stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptides and or polypeptides from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Modifications can include any modification to the sugar, the nucleobase, the intemucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, the stagger element is present in the circular polyribonucleotide in other forms. For example, in some exemplary circular polyribonucleotides, a stagger element includes a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence.

In some embodiments, the first stagger element includes a termination element and separates an expression product of the first expression sequence from an expression product of its succeeding expression sequences, thereby creating discrete expression products. In some cases, the circular polyribonucleotide including the first stagger element upstream of the first translation initiation sequence of the succeeding sequence in the circular polyribonucleotide is continuously translated, while a corresponding circular polyribonucleotide including a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element includes a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences.

In some embodiments, the first stagger element separates one round expression product of the first expression sequence from the next round expression product of the first expression sequences, thereby creating discrete expression products. In some cases, the circular polyribonucleotide including the first stagger element upstream of the first translation initiation sequence of the first expression sequence in the circular polyribonucleotide is continuously translated, while a corresponding circular polyribonucleotide including a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide includes more than one expression sequence.

Examples of stagger elements are described in paragraphs [0172]-[0175] of International Patent Publication No. WO2019/118919, which is hereby incorporated by reference in its entirety.

### Non-Coding Sequences

In some embodiments, the polyribonucleotide described herein (e.g., the polyribonucleotide cargo of the polyribonucleotide) includes one or more non-coding sequence, e.g., a sequence that does not encode the expression of polypeptide. In some embodiments, the polyribonucleotide includes two, three, four, five, six, seven, eight, nine, ten or more than ten non-coding sequences. In some embodiments, the polyribonucleotide does not encode a polypeptide expression sequence.

Noncoding sequences can be natural or synthetic sequences. In some embodiments, a noncoding sequence can alter cellular behavior, such as e.g., lymphocyte behavior. In some embodiments, the noncoding sequences are antisense to cellular RNA sequences.

In some embodiments, the polyribonucleotide includes regulatory nucleic acids that are RNA or RNA-like structures typically from about 5-500 base pairs (depending on the specific RNA structure (e.g., miRNA 5-30 bps, lncRNA 200-500 bps) and may have a nucleobase sequence identical (complementary) or nearly identical (substantially complementary) to a coding sequence in an expressed target gene within the cell. In embodiments, the circular polyribonucleotide includes regulatory nucleic acids that encode an RNA precursor that can be processed to a smaller RNA, e.g., a miRNA precursor, which can be from about 50 to about 1000 bp, that can be processed to a smaller miRNA intermediate or a mature miRNA.

Long non-coding RNAs (lncRNA) are defined as non-protein coding transcripts longer than 100 nucleotides. Many lncRNAs are characterized as tissue specific. Divergent lncRNAs that are transcribed in the opposite direction to nearby protein-coding genes include a significant proportion (e.g., about 20% of total lncRNAs in mammalian genomes) and possibly regulate the transcription of the nearby gene. In one embodiment, the polyribonucleotide provided herein includes a sense strand of a lncRNA. In one embodiment, the polyribonucleotide provided herein includes an antisense strand of a lncRNA.

In embodiments, the polyribonucleotide encodes a regulatory nucleic acid that is substantially complementary, or fully complementary, to all or to at least one fragment of an endogenous gene or gene product (e.g., mRNA). In embodiments, the regulatory nucleic acids complement sequences at the boundary between introns and exons, in between exons, or adjacent to an exon, to prevent the maturation of newly generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof. In some embodiments, the regulatory nucleic acid includes a protein-binding site that can bind to a protein that participates in regulation of expression of an endogenous gene or an exogenous gene.

In embodiments, the polyribonucleotide encodes a regulatory RNA that hybridizes to a transcript of interest wherein the regulatory RNA has a length of from about 5 to 30 nucleotides, from about 10 to 30 nucleotides, or about 11, 12, 13, 14, 15, 16, 17, 18, 19, 20, 21, 22, 23, 24, 25, 26, 27, 28, 29, 30 or more than 30 nucleotides. In embodiments, the degree of sequence identity of the regulatory RNA to the targeted transcript is at least 75%, at least 80%, at least 85%, at least 90%, or at least 95%.

In embodiments, the polyribonucleotide encodes a microRNA (miRNA) molecule identical to about 5 to about 25 contiguous nucleotides of a target gene or encodes a precursor to that miRNA. In some embodiments, the miRNA has a sequence that allows the mRNA to recognize and bind to a specific target mRNA. In embodiments, miRNA sequence commences with the dinucleotide AA, includes a GC-content of about 30-70% (about 30-60%, about 40-60%, or about 45%-55%), and does not have a high percentage identity to any nucleotide sequence other than the target in the genome of the subject (e.g., a mammal) in which it is to be introduced, for example as determined by standard BLAST search.

In some embodiments, the polyribonucleotide includes at least one miRNA (or miRNA precursor), e.g., 2, 3, 4, 5, 6, or more miRNAs or miRNA precursors. In some embodiments, the polyribonucleotide includes a sequence that encodes a miRNA (or its precursor) having at least about 75%, 80%, 85%, 90% 95%, 96%, 97%, 98%, or 99% or 100% nucleotide sequence complementarity to a target sequence.

siRNAs and shRNAs resemble intermediates in the processing pathway of the endogenous microRNA (miRNA) genes. In some embodiments, siRNAs can function as miRNAs and vice versa. MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation. Known miRNA binding sites are within mRNA 3′ UTRs; miRNAs target sites with near-perfect complementarity to nucleotides 2-8 from the miRNA's 5′ end. This region is known as the seed region. Because mature siRNAs and miRNAs are interchangeable, exogenous siRNAs downregulate mRNAs with seed complementarity to the siRNA. Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs.

### Protein-Binding Sequences

In some embodiments, a circular polyribonucleotide includes one or more protein binding sites that enable a protein, e.g., a ribosome, to bind to an internal site in the RNA sequence. By engineering protein binding sites, e.g., ribosome binding sites, into the circular polyribonucleotide, the circular polyribonucleotide may evade or have reduced detection by the host's immune system, have modulated degradation, or modulated translation, by masking the circular polyribonucleotide from components of the host's immune system.

In some embodiments, a circular polyribonucleotide includes at least one immunoprotein binding site, for example to evade immune responses, e.g., CTL (cytotoxic T lymphocyte) responses. In some embodiments, the immunoprotein binding site is a nucleotide sequence that binds to an immunoprotein and aids in masking the circular polyribonucleotide as exogenous. In some embodiments, the immunoprotein binding site is a nucleotide sequence that binds to an immunoprotein and aids in hiding the circular polyribonucleotide as exogenous or foreign.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present disclosure, internal initiation (i.e., cap-independent) of translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences including a ribosome binding site, e.g., an initiation codon.

Natural 5′ UTRs bear features which play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 125), where R is a purine (adenine or guanine) three bases upstream of the start codon (AUG), which is followed by another ‘G’. 5′ UTR also have been known to form secondary structures which are involved in elongation factor binding.

In some embodiments, a circular polyribonucleotide encodes a protein binding sequence that binds to a protein. In some embodiments, the protein binding sequence targets or localizes the circular polyribonucleotide to a specific target. In some embodiments, the protein binding sequence specifically binds an arginine-rich region of a protein.

In some embodiments, the protein binding site includes, but is not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO-, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF 5, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

### Spacer Sequences

In some embodiments, a polyribonucleotide described herein includes one or more spacer sequences. A spacer refers to any contiguous nucleotide sequence (e.g., of one or more nucleotides) that provides distance or flexibility between two adjacent polynucleotide regions. Spacers may be present in between any of the nucleic acid elements described herein. Spacer may also be present within a nucleic acid element described herein.

The spacer may be, e.g., at least 5 (e.g., at least 10, at least 15, at least 20) ribonucleotides in length. In some embodiments, each spacer region is at least 5 (e.g., at least 10, at least 15, at least 20) ribonucleotides in length. Each spacer region may be, e.g., from 5 to 500 (e.g., 10, 20, 30, 40, 50, 60, 70, 80, 90, 100, 150, 200, 250, 300, 350, 400, 450, or 500) ribonucleotides in length. The first spacer region, the second spacer region, or the first spacer region and the second spacer region may include a polyA sequence. The first spacer region, the second spacer region, or the first spacer region and the second spacer region may include a polyA-C sequence. In some embodiments, the first spacer region, the second spacer region, or the first spacer region and the second spacer region includes a polyA-G sequence. In some embodiments, the first spacer region, the second spacer region, or the first spacer region and the second spacer region includes a polyA-T sequence. In some embodiments, the first spacer region, the second spacer region, or the first spacer region and the second spacer region includes a random sequence.

Spacers may also be present within a nucleic acid region described herein. For example, a polynucleotide cargo region may include one or multiple spacers. Spacers may separate regions within the polynucleotide cargo.

In some embodiments, the spacer sequence can be, for example, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is from 20 to 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The spacer sequences can be polyA sequences, polyA-C sequences, polyC sequences, or poly-U sequences.

In some embodiments, the spacer sequences can be polyA-T, polyA-C, polyA-G, or a random sequence.

A spacer sequence may be used to separate an IRES from adjacent structural elements to maintain the structure and function of the IRES or the adjacent element. A spacer can be specifically engineered depending on the IRES. In some embodiments, an RNA folding computer software, such as RNAFold, can be utilized to guide designs of the various elements of the vector, including the spacers.

In some embodiments, the polyribonucleotide includes a 5′ spacer sequence. In some embodiments, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence. In some embodiments, the 5′ spacer sequence includes a polyA-G sequence. In some embodiments, the 5′ spacer sequence includes a polyA-T sequence. In some embodiments, the 5′ spacer sequence includes a random sequence.

In some embodiments, the polyribonucleotide includes a 3′ spacer sequence. In some embodiments, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is from 20 to 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence. In some embodiments, the 5′ spacer sequence includes a polyA-G sequence. In some embodiments, the 5′ spacer sequence includes a polyA-T sequence. In some embodiments, the 5′ spacer sequence includes a random sequence.

In one embodiment, the polyribonucleotide includes a 5′ spacer sequence, but not a 3′ spacer sequence. In another embodiment, the polyribonucleotide includes a 3′ spacer sequence, but not a 5′ spacer sequence. In another embodiment, the polyribonucleotide includes neither a 5′ spacer sequence, nor a 3′ spacer sequence. In another embodiment, the polyribonucleotide does not include an IRES sequence. In a further embodiment, the polyribonucleotide does not include an IRES sequence, a 5′ spacer sequence or a 3′ spacer sequence.

In some embodiments, the spacer sequence includes at least 3 ribonucleotides, at least 4 ribonucleotides, at least 5 ribonucleotides, at least about 8 ribonucleotides, at least about 10 ribonucleotides, at least about 12 ribonucleotides, at least about 15 ribonucleotides, at least about 20 ribonucleotides, at least about 25 ribonucleotides, at least about 30 ribonucleotides, at least about 40 ribonucleotides, at least about 50 ribonucleotides, at least about 60 ribonucleotides, at least about 70 ribonucleotides, at least about 80 ribonucleotides, at least about 90 ribonucleotides, at least about 100 ribonucleotides, at least about 120 ribonucleotides, at least about 150 ribonucleotides, at least about 200 ribonucleotides, at least about 250 ribonucleotides, at least about 300 ribonucleotides, at least about 400 ribonucleotides, at least about 500 ribonucleotides, at least about 600 ribonucleotides, at least about 700 ribonucleotides, at least about 800 ribonucleotides, at least about 900 ribonucleotides, or at least about 1,000 ribonucleotides.

### Bioreactors

In some embodiments, any method of purifying a polyribonucleotide (e.g., circular polyribonucleotide) described herein may be performed in a bioreactor. A bioreactor refers to any vessel in which a chemical or biological process is carried out which involves organisms or biochemically active substances derived from such organisms. Bioreactors may be compatible with the cell-free methods for purifying or producing circular RNA described herein. A vessel for a bioreactor may include a culture flask, a dish, or a bag that may be individual use (disposable), autoclavable, or sterilizable. A bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest.

Some methods of the present disclosure are directed to large-scale production of polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 5 L to 50 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, a bioreactor may produce at least 1 g of RNA. In some embodiments, a bioreactor may produce 1-200 g of RNA (e.g., 1-10 g, 1-20 g, 1-50 g, 10-50 g, 10-100 g, 50-100 g, of 50-200 g of RNA). In some embodiments, the amount produced is measured per liter (e.g., 1-200 g per liter), per batch or reaction (e.g., 1-200 g per batch or reaction), or per unit time (e.g., 1-200 g per hour or per day).

In some embodiments, more than one bioreactor may be utilized in series to increase the production capacity (e.g., one, two, three, four, five, six, seven, eight, or nine bioreactors may be used in series).

### Methods of Use

In some embodiments, the polyribonucleotides (e.g., circular polyribonucleotides) made as described herein are used as effectors in therapy or agriculture.

For example, a polyribonucleotide purified by the methods described herein may be administered to a subject (e.g., in a pharmaceutical, veterinary, or agricultural composition). In some embodiments, the subject is a vertebrate animal (e.g., mammal, bird, fish, reptile, or amphibian). In some embodiments, the subject is a human. In some embodiments, the subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the subject is an invertebrate such as an arthropod (e.g., insects, arachnids, crustaceans), a nematode, an annelid, a helminth, or a mollusk. In embodiments, the subject is an invertebrate agricultural pest or an invertebrate that is parasitic on an invertebrate or vertebrate host. In embodiments, the subject is a plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the subject is a eukaryotic alga (unicellular or multicellular). In embodiments, the subject is a plant of agricultural or horticultural importance, such as row crop plants, fruit-producing plants and trees, vegetables, trees, and ornamental plants including ornamental flowers, shrubs, trees, groundcovers, and turf grasses.

In some embodiments, the disclosure provides a method of modifying a subject by providing to the subject a composition or formulation described herein. In some embodiments, the composition or formulation is or includes a nucleic acid molecule (e.g., a DNA molecule or an RNA molecule described herein), and the polynucleotide is provided to a eukaryotic subject. In some embodiments, the composition or formulation is or includes or a eukaryotic or prokaryotic cell including a nucleic acid described herein.

In some embodiments, the disclosure provides a method of treating a condition in a subject in need thereof by providing to the subject a composition or formulation described herein. In some embodiments, the composition or formulation is or includes a nucleic acid molecule (e.g., a DNA molecule or a polyribonucleotide described herein), and the polynucleotide is provided to a eukaryotic subject. In some embodiments, the composition or formulation is or includes a eukaryotic or prokaryotic cell including a nucleic acid described herein.

In some embodiments, the disclosure provides a method of providing a polyribonucleotide (e.g., circular polyribonucleotide) to a subject by providing a eukaryotic or prokaryotic cell include a polynucleotide described herein to the subject.

### Formulations

In some embodiments of the present disclosure a polyribonucleotide (e.g., a circular polyribonucleotide) described herein may be formulated in composition, e.g., a composition for delivery to a cell, a plant, an invertebrate animal, a non-human vertebrate animal, or a human subject, e.g., an agricultural, veterinary, or pharmaceutical composition. In some embodiments, the polyribonucleotide is formulated in a pharmaceutical composition. In some embodiments, a composition includes a polyribonucleotide and a diluent, a carrier, an adjuvant, or a combination thereof. In a particular embodiment, a composition includes a polyribonucleotide described herein and a carrier or a diluent free of any carrier. In some embodiments, a composition including a polyribonucleotide with a diluent free of any carrier is used for naked delivery of the polyribonucleotide (e.g., circular polyribonucleotide) to a subject.

Pharmaceutical compositions may optionally include one or more additional active substances, e.g., therapeutically and/or prophylactically active substances. Pharmaceutical compositions may optionally include an inactive substance that serves as a vehicle or medium for the compositions described herein (e.g., compositions including circular polyribonucleotides, such as any one of the inactive ingredients approved by the United States Food and Drug Administration (FDA) and listed in the Inactive Ingredient Database). Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference). Non-limiting examples of an inactive substance include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the reference criterion for the amount of linear polyribonucleotide molecules present in the preparation is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 650 mg/mL, 700 mg/mL, 700 ng/mL, 750 mg/mL, 800 ng/mL, 850 ng/mL, 900 ng/mL, 950 ng/mL, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

In some embodiments, the reference criterion for the amount of circular polyribonucleotide molecules present in the preparation is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the pharmaceutical preparation.

In some embodiments, the reference criterion for the amount of linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.

In some embodiments, the reference criterion for the amount of nicked polyribonucleotide molecules present in the preparation is no more than 0.5% (w/w), 1% (w/w), 2% (w/w), 5% (w/w), 10% (w/w), or 15% (w/w) nicked polyribonucleotide molecules of the total ribonucleotide molecules in the pharmaceutical preparation.

In some embodiments, the reference criterion for the amount of combined nicked and linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the pharmaceutical preparation. In some embodiments, a pharmaceutical preparation is an intermediate pharmaceutical preparation of a final circular polyribonucleotide drug product. In some embodiments, a pharmaceutical preparation is a drug substance or active pharmaceutical ingredient (API). In some embodiments, a pharmaceutical preparation is a drug product for administration to a subject.

In some embodiments, a preparation of circular polyribonucleotides is (before, during or after the reduction of linear RNA) further processed to remove DNA, protein contamination (e.g., cell protein such as a host cell protein or protein process impurities), endotoxin, mononucleotide molecules, and/or a process-related impurity.

### Salts

In some cases, a composition or pharmaceutical composition provided herein includes one or more salts. For controlling the tonicity, a physiological salt such as sodium salt can be included a composition provided herein. Other salts can include potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methanesulfonic acid, p-toluene sulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can include one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Detergents/Surfactants

A composition or pharmaceutical composition provided herein can include one or more detergents and/or surfactants, depending on the intended administration route, e.g., polyoxymethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-I,2-ethanediyl) groups, e.g., octoxynol-9 (Triton X-100, or t-octylphenoxypolyethoxyethanol); (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Diluents

In some embodiments, a composition of the disclosure includes a polyribonucleotide and a diluent. In some embodiments, a composition of the disclosure includes a linear polyribonucleotide and a diluent.

A diluent can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans to render the compositions suitable for administration to various animals is well understood, and the ordinarily skilled veterinary pharmacologist can design and/or perform such modification with merely ordinary, if any, experimentation.

In some embodiments, the polyribonucleotide (e.g., circular polyribonucleotide) is delivered as a naked delivery formulation, such as including a diluent. A naked delivery formulation delivers a polyribonucleotide, to a cell without the aid of a carrier and without modification or partial or complete encapsulation of the polyribonucleotide, capped polyribonucleotide, or complex thereof.

A naked delivery formulation is a formulation that is free from a carrier and wherein the polyribonucleotide (e.g., circular polyribonucleotide) is without a covalent modification that binds a moiety that aids in delivery to a cell or without partial or complete encapsulation of the polyribonucleotide. In some embodiments, a polyribonucleotide without a covalent modification that binds a moiety that aids in delivery to a cell is a polyribonucleotide that is not covalently bound to a protein, small molecule, a particle, a polymer, or a biopolymer. A polyribonucleotide without covalent modification that binds a moiety that aids in delivery to a cell does not contain a modified phosphate group. For example, a polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of transfection reagents, cationic carriers, carbohydrate carriers, nanoparticle carriers, or protein carriers. In some embodiments, a naked delivery formulation is free from phytoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, I,2-Dioleoyl-3-Trimethylammonium-Propane(DOTAP), N-[1-(2,3-dioleoyloxy)propyl]-N,N,N-trimethylammonium chloride (DOTMA), I-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B—[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N—(I,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carriers

In some embodiments, a composition of the disclosure includes a circular polyribonucleotide and a carrier. In some embodiments, a composition of the disclosure includes a linear polyribonucleotide and a carrier.

In certain embodiments, a composition includes a circular polyribonucleotide as described herein in a vesicle or other membrane-based carrier. In certain embodiments, a composition includes a linear polyribonucleotide as described herein in a vesicle or other membrane-based carrier.

In other embodiments, a composition includes the circular polyribonucleotide in or via a cell, vesicle or other membrane-based carrier. In other embodiments, a composition includes the linear polyribonucleotide in or via a cell, vesicle or other membrane-based carrier. In one embodiment, a composition includes the circular polyribonucleotide in liposomes or other similar vesicles. In one embodiment, a composition includes the linear polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, JOURNAL OF DRUG DELIVERY, vol. 2011, Article ID 469679, 12 pages, 2011. doi:10.1155/2011/469679 for review).

Vesicles can be made from several distinct types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, JOURNAL OF DRUG DELIVERY, vol. 2011, Article ID 469679, 12 pages, 2011. doi:10.1155/2011/469679 for review). Extruded lipids can be prepared by extruding through filters of decreasing size, as described in Templeton et al., NATURE BIOTECH, 15:647-652, 1997, the teachings of which relating to extruded lipid preparation are incorporated herein by reference.

In certain embodiments, a composition of the disclosure includes a polyribonucleotide and lipid nanoparticles, for example lipid nanoparticles described herein. In certain embodiments, a composition of the disclosure includes a linear polyribonucleotide and lipid nanoparticles. Lipid nanoparticles are another example of a carrier that provides a biocompatible and biodegradable delivery system for a polyribonucleotide molecule as described herein. Lipid nanoparticles are another example of a carrier that provides a biocompatible and biodegradable delivery system for a linear polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are a key component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, NANOMATERIALS 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the polyribonucleotide, or a protein covalently linked to the linear polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, I,2-Dioleoyl-3-Trimethylammonium-Propane (DOTAP), N-[1-(2,3-dioleoyloxy)propyl]-N,N,N-trimethylammonium chloride (DOTMA), I-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-I-propanaminium trifluoroacetate (DOSPA), 3B—[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N—(I,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a composition or preparation described herein. Exosomes can be used as drug delivery vehicles for a linear polyribonucleotide composition or preparation described herein. For a review, see Ha et al. July 2016. Acta Pharmaceutica Sinica B. Volume 6, Issue 4, Pages 287-96; doi.org/10.1016/j.apsb.2016.02.001.

Ex vivo differentiated red blood cells can also be used as a carrier for a composition or preparation described herein. Ex vivo differentiated red blood cells can also be used as a carrier for a linear polyribonucleotide composition or preparation described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. PROC NATL ACAD SCI USA. 111(28): 10131-10136; U.S. Pat. No. 9,644,180; Huang et al. 2017. NATURE COMMUNICATIONS 8: 423; Shi et al. 2014. PROC NATL ACAD SCI USA. 111(28): 10131-10136.

Fusosome compositions, e.g., as described in International Patent Publication No. WO2018/208728, can also be used as carriers to deliver a polyribonucleotide molecule described herein. Fusosome compositions, e.g., as described in WO2018/208728, can also be used as carriers to deliver a linear polyribonucleotide molecule described herein.

Virosomes and virus-like particles (VLPs) can also be used as carriers to deliver a polyribonucleotide molecule described herein to targeted cells. Virosomes and virus-like particles (VLPs) can also be used as carriers to deliver a linear polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver the composition or preparation described herein. Plant nanovesicles and plant messenger packs (PMPs) can also be used as carriers to deliver a linear polyribonucleotide composition or preparation described herein.

Microbubbles can also be used as carriers to deliver a polyribonucleotide molecule described herein. Microbubbles can also be used as carriers to deliver a linear polyribonucleotide molecule described herein. See, e.g., U.S. Pat. No. 7,115,583; Beeri, R. et al., CIRCULATION. 2002 Oct. 1; 106(14):1756-1759; Bez, M. et al., NAT PROTOC. 2019 April; 14(4): 1015-1026; Hernot, S. et al., ADV DRUG DELIV REV. 2008 Jun. 30; 60(10): 1153-1166; Rychak, J. J. et al., ADV DRUG DELIV REV. 2014 June; 72: 82-93. In some embodiments, microbubbles are albumin-coated perfluorocarbon microbubbles.

Silk fibroin can also be used as a carrier to deliver the compositions and preparations described herein. See, e.g., Boopathy, A. V. et al., PNAS. 116.33 (2019): 16473-1678; and He, H. et al., ACS BIOMATER. SCI. ENG. 4.5 (2018): 1708-1715.

The carrier including the polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the polyribonucleotide into a cell. Deposition of the polyribonucleotide into certain cell types may favor different particle sizes. For example, the particle size may be optimized for deposition of the polyribonucleotide into antigen presenting cells. The particle size may be optimized for deposition of the polyribonucleotide into dendritic cells. Additionally, the particle size may be optimized for depositions of the polyribonucleotide into draining lymph node cells.

### Lipid Nanoparticles

In some embodiments, a composition of the disclosure includes a circular polyribonucleotide and lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as I-(monomethoxy-polyethylene glycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-I-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

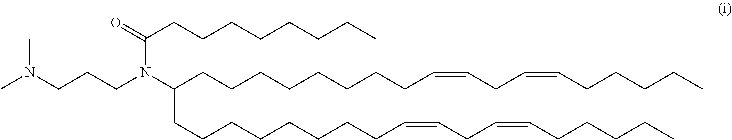 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., a circular polyribonucleotide, a linear polyribonucleotide) composition described herein to cells.

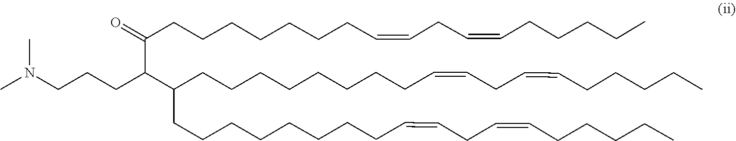 [SMILES: CCCCC/C=C\C/C=C\CCCCCCCCC(CCCCCCCC/C=C\C/C=C\CCCCC)C(CCCN(C)C)C(=O)CCCCCCC/C=C\C/C=C\CCCCC]

In some embodiments an LNP including Formula (ii) is used to deliver a polyribonucleotide (e.g., a circular polyribonucleotide, a linear polyribonucleotide) composition described herein to cells.

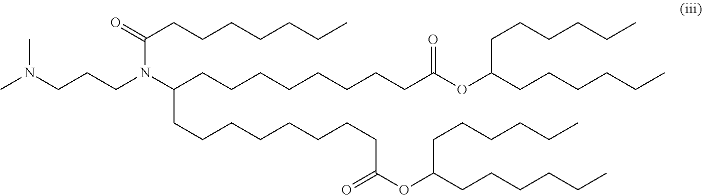 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., a circular polyribonucleotide, a linear polyribonucleotide) composition described herein to cells.

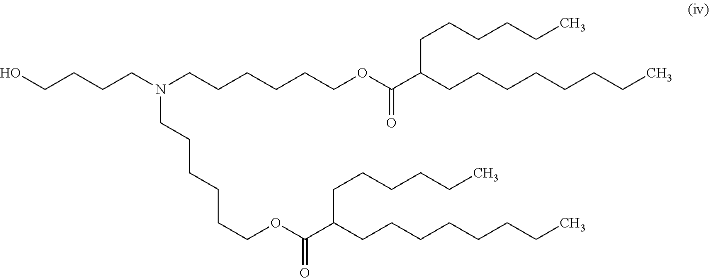 [SMILES: CCCCCCCCC(CCCCCC)C(=O)OCCCCCCN(CCCCO)CCCCCCOC(=O)C(CCCCCC)CCCCCCCC]

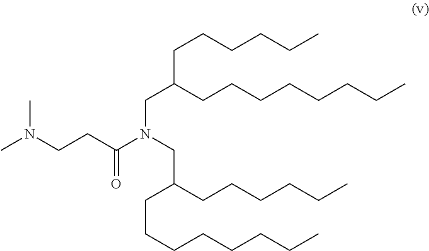 [SMILES: CCCCCCCCC(CCCCCC)CN(CC(CCCCCC)CCCCCCCC)C(=O)CCN(C)C]

In some embodiments an LNP including Formula (v) is used to deliver a polyribonucleotide (e.g., a circular polyribonucleotide, a linear polyribonucleotide) composition described herein to cells.

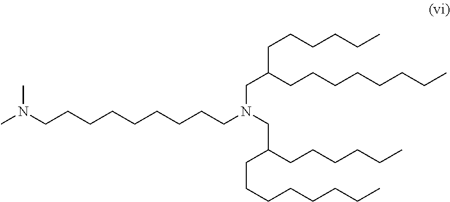 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., a circular polyribonucleotide, a linear polyribonucleotide) composition described herein to cells.

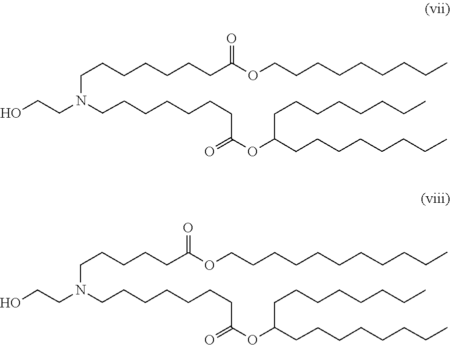 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., a circular polyribonucleotide, a linear polyribonucleotide) composition described herein to cells.

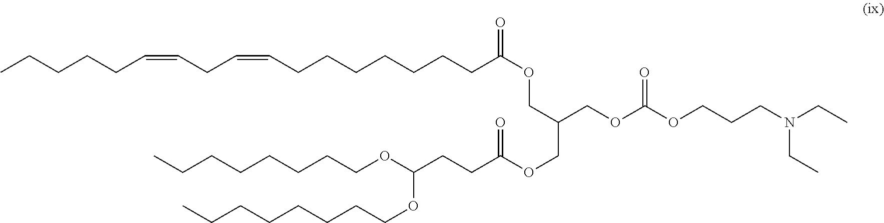 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., a circular polyribonucleotide, a linear polyribonucleotide) composition described herein to cells.

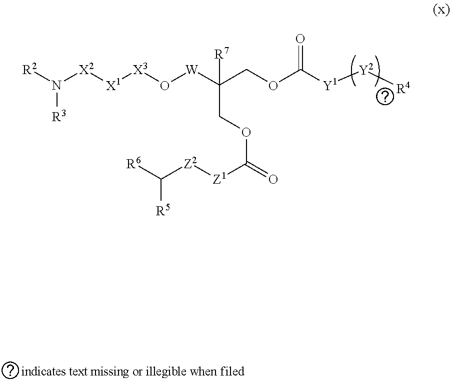

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with RWand the nitrogen atom to which they are attached form a 5-, 6-, or 7-membered ring, Y¹ is C2-12 alkylene, Y² is selected from

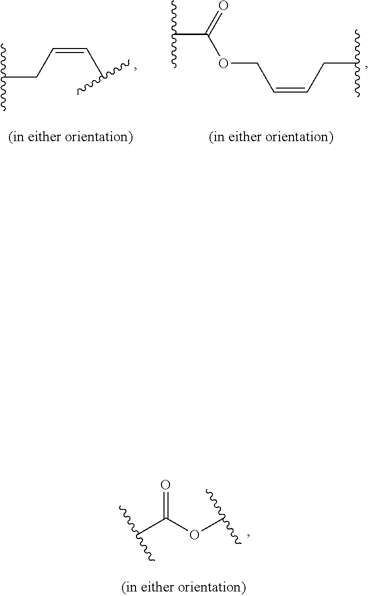 [SMILES: */C=C\C* ; *C/C=C\COC(*)=O ; *OC(*)=O]

n is 0 to 3, R⁴ is C1-15 alkyl, Z¹ is C1-6 alkylene or a direct bond,
Z² is

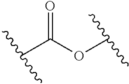 [SMILES: *OC(*)=O]

(in either orientation) or absent, provided that if Z¹ is a direct bond, Z² is absent,
R⁵ is C5-9 alkyl or C6-10 alkoxy, R⁶ is C5-9 alkyl or C6-10 alkoxy, W is methylene or a direct bond, and R⁷ is H or for a salt thereof, provided that if R³ and R² are C2 alkyls, X¹ is 0, X² is linear C3 alkylene, X³ is C(═O), Y¹ is linear Ce alkylene, (Y²)n-R⁴ is

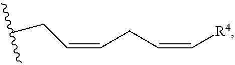

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., a circular polyribonucleotide, a linear polyribonucleotide) composition described herein to cells.

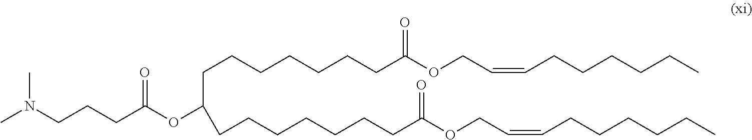 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., a circular polyribonucleotide, a linear polyribonucleotide) composition described herein to cells.

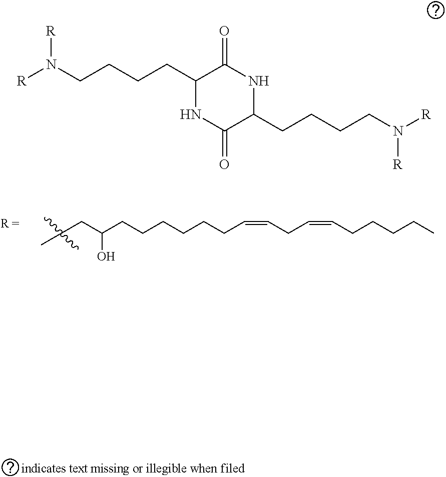

where R=

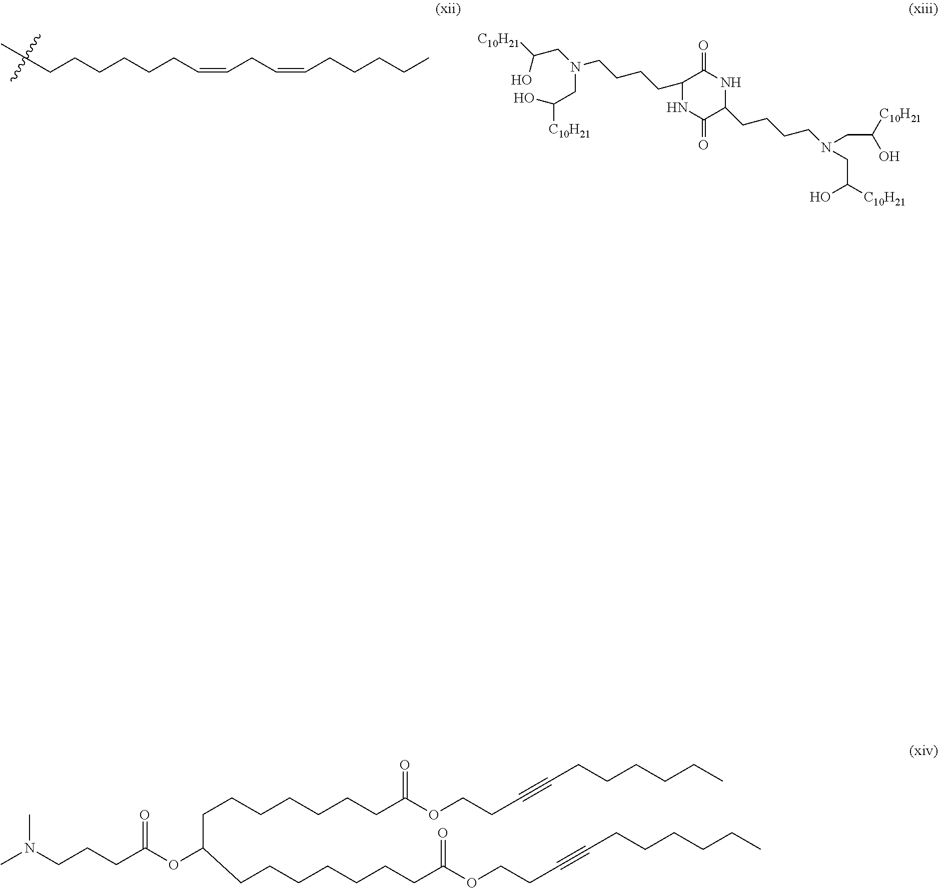

In some embodiments an LNP includes a compound of Formula (xiii) and a compound of Formula (xiv).

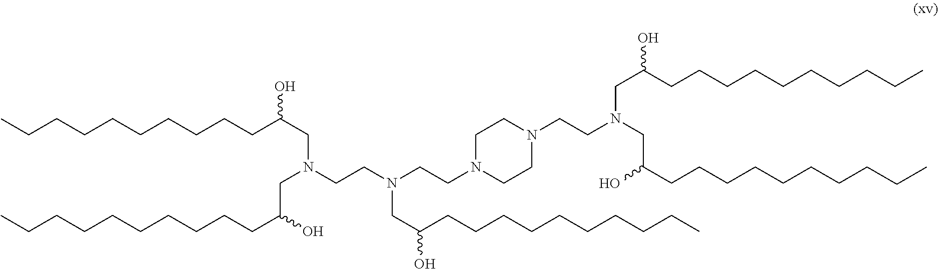 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., a circular polyribonucleotide, a linear polyribonucleotide) composition described herein to cells.

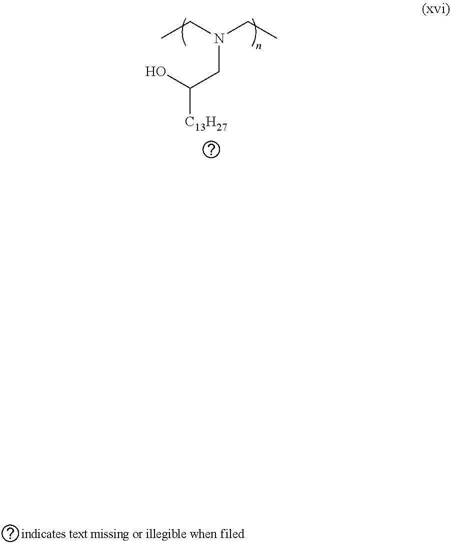

In some embodiments an LNP including a formulation of Formula (xvi) is used to deliver a polyribonucleotide (e.g., a circular polyribonucleotide, a linear polyribonucleotide) composition described herein to cells.

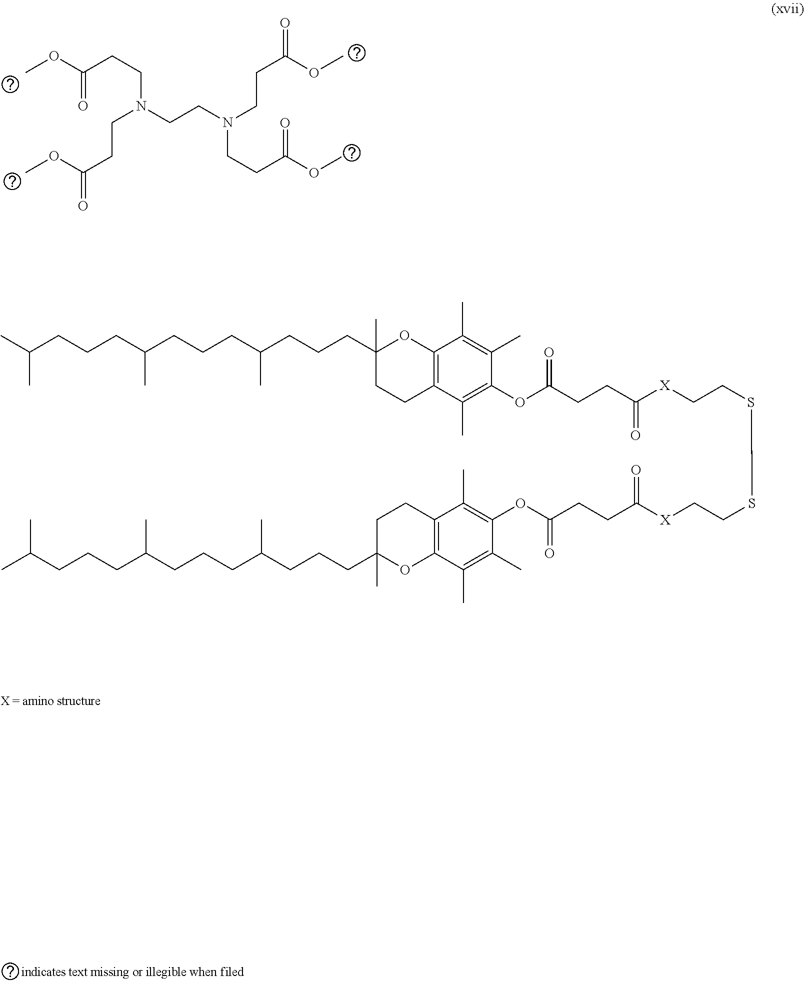

where X=

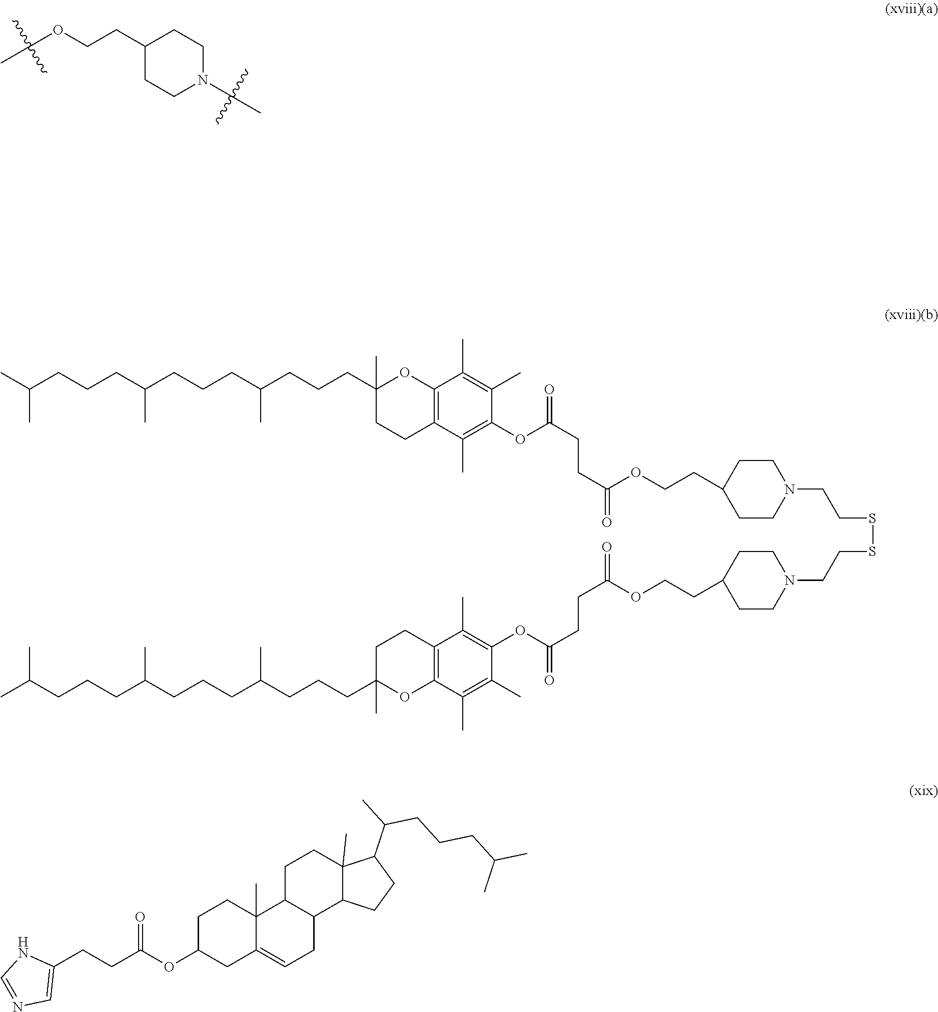 [SMILES: *OCCC1CCN(*)CC1 ; Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2 ; CC(C)CCCC(C)C1CCC2C3CC=C4CC(OC(=O)CCc5cnc[nH]5)CCC4(C)C3CCC12C]

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

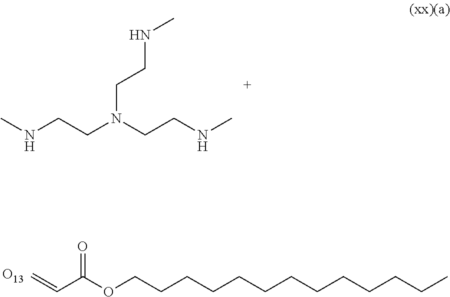

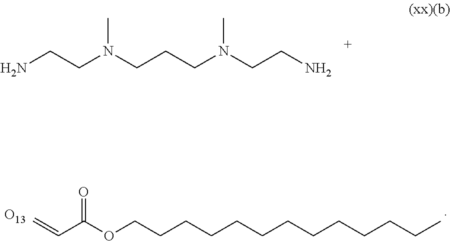 [SMILES: CN(CCN)CCCN(C)CCN ; "O13" ; C=CC(=O)OCCCCCCCCCCCCC]

In some embodiments an LNP including Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

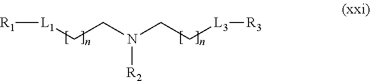

wherein
   each n is independently an integer from 2-15; L₁ and L₃ are each independently —OC(O)—* or —C(O)O—*, wherein “*” indicates the attachment point to R₁ or R₃;
   R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxy alkyl, hydroxyalkylaminoalkyl, amino alkyl, alkylaminoalkyl, dialkylamino alkyl, (heterocyclyl)(alkyl)aminoalkyl, heterocyclyl, heteroaryl, alkyl heteroaryl, alkynyl, alkoxy, amino, dialkylamino, aminoalkylcarbonylamino, aminocarbonyl alkylamino, (aminocarbonylalkyl)(alkyl)amino, alkenylcarbonylamino, hydroxycarbonyl, alkyloxy carbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenyl carbonyl, alkynyl carbonyl, alkyl sulfoxide, alkylsulfoxidealkyl, alkyl sulfonyl, and alkyl sulfone alkyl; and
   R₂ is selected from a group consisting of:

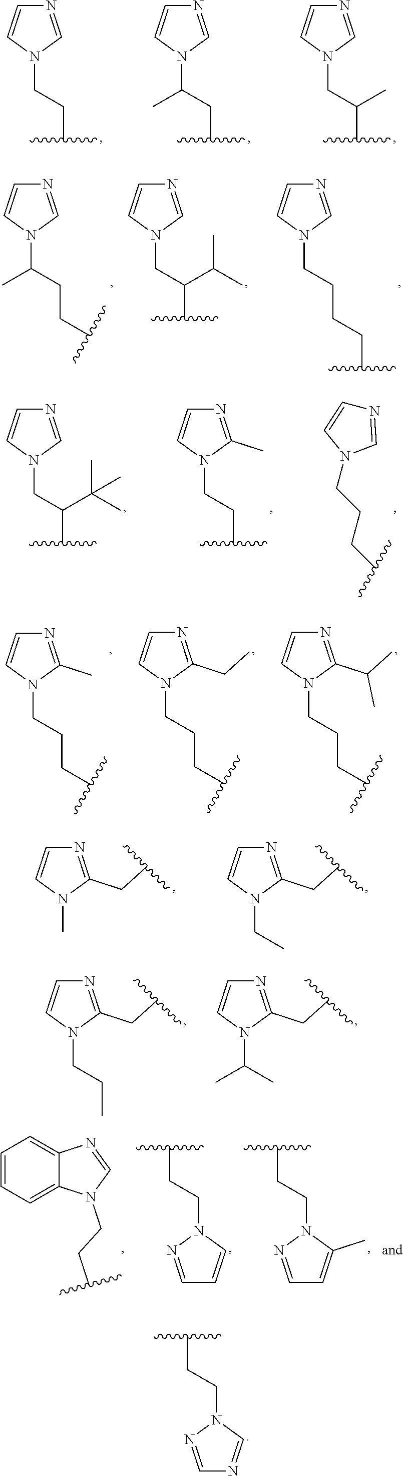

In some embodiments an LNP including Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

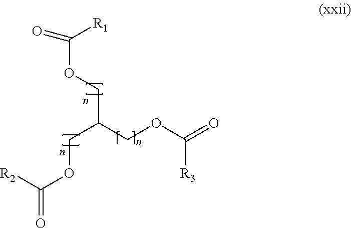

wherein
   each n is independently an integer from 1-15;
   R₁ and R₂ are each independently selected from a group consisting of:

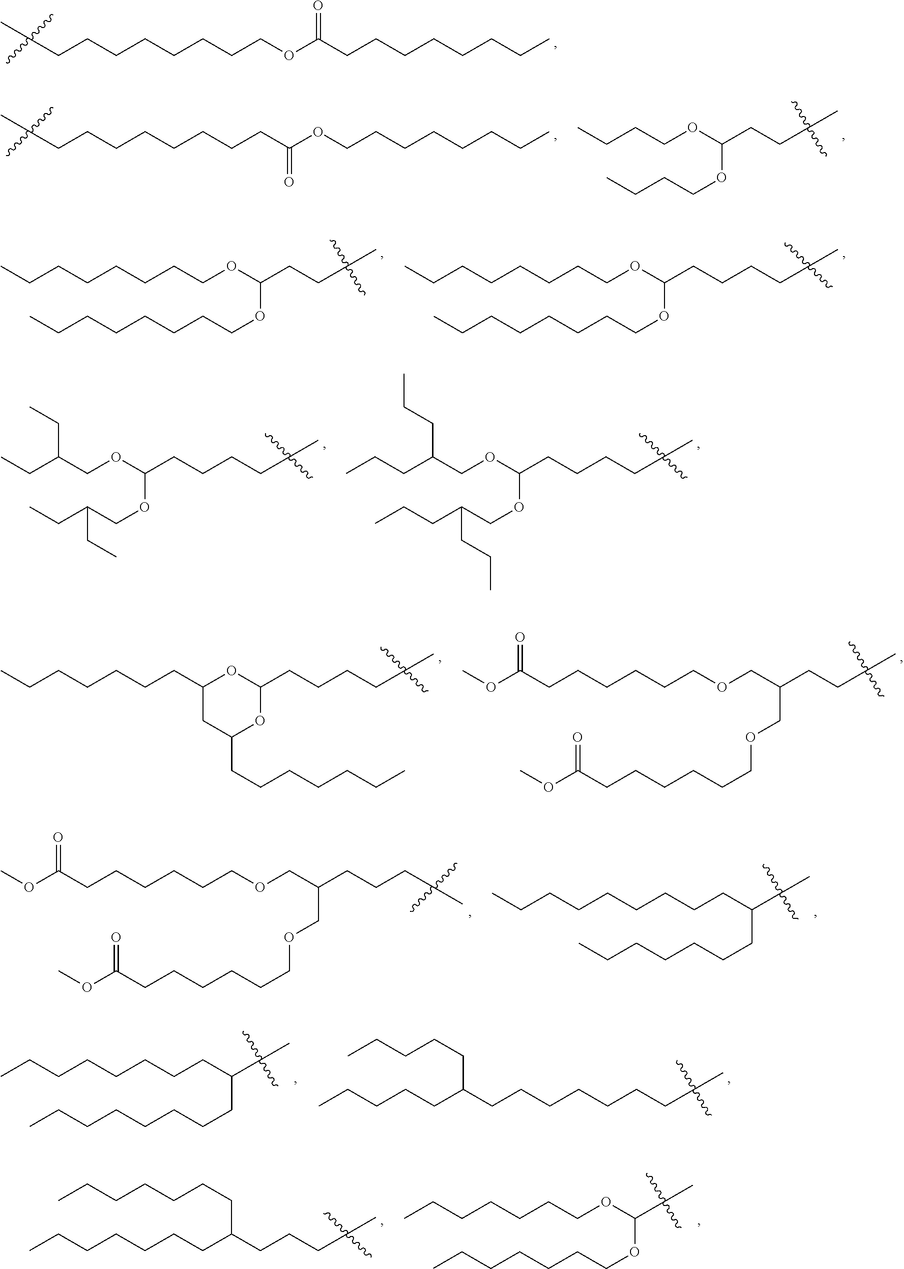

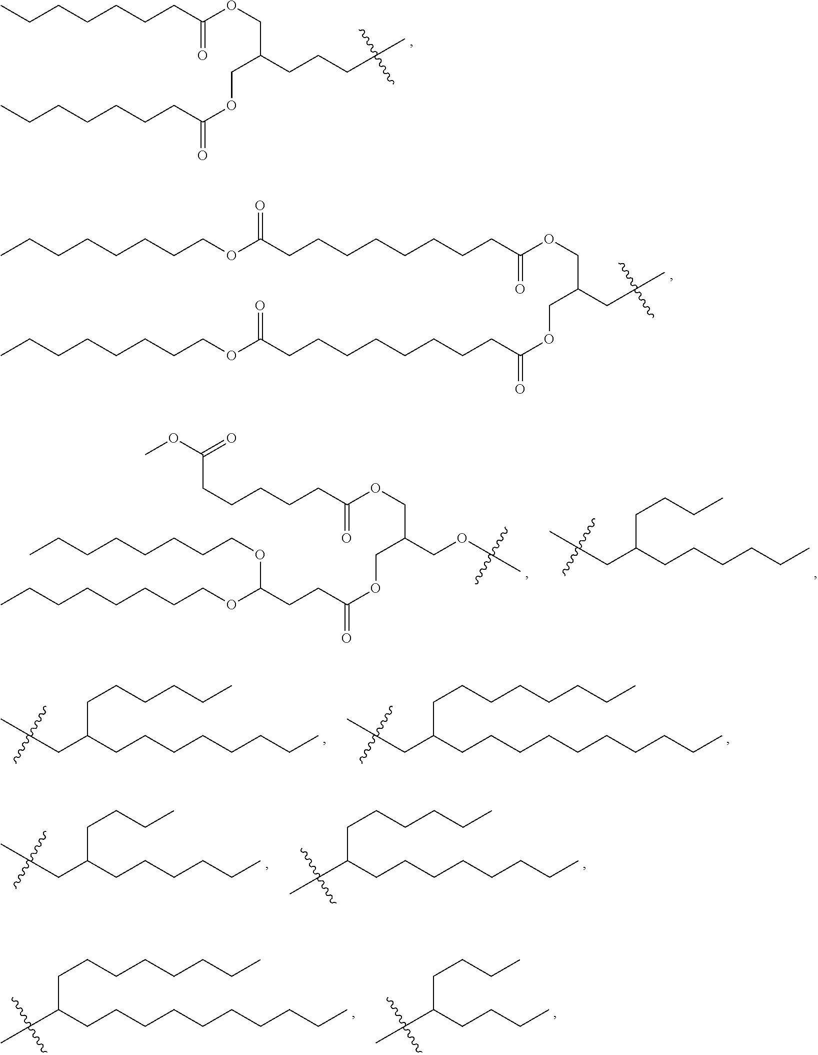

R₃ is selected from a group consisting of:

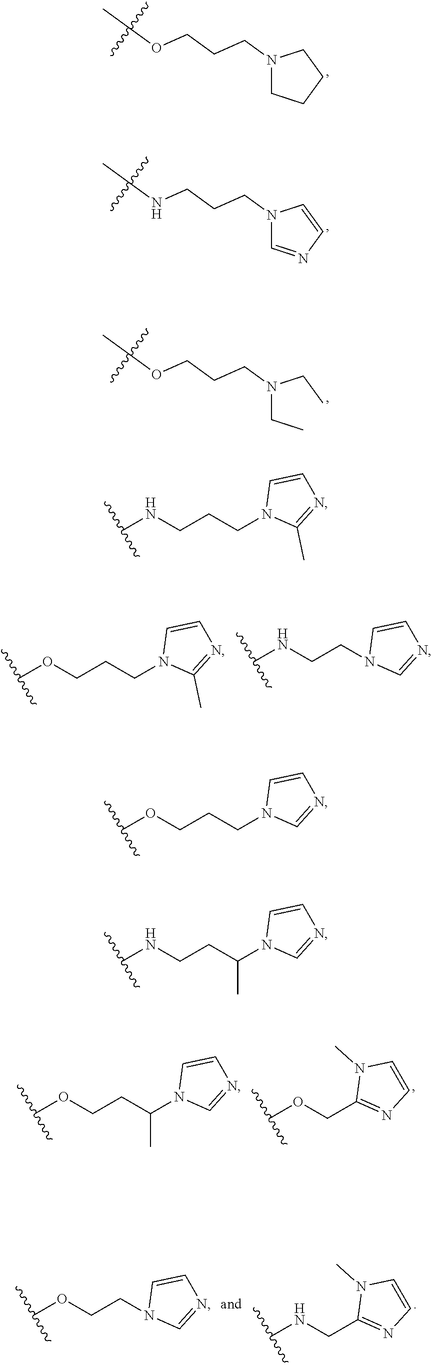

In some embodiments an LNP including Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

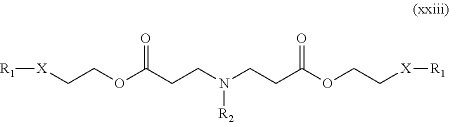

wherein
   X is selected from —O—, —S—, or —OC(O)—*, wherein * indicates the attachment point to R₁;
   R₁ is selected from a group consisting of:

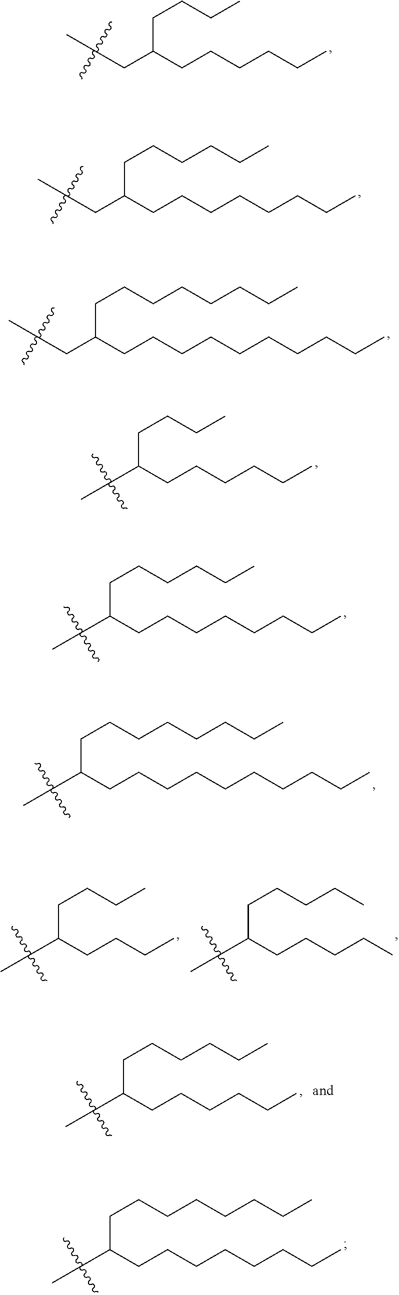

and R₂ is selected from a group consisting of:

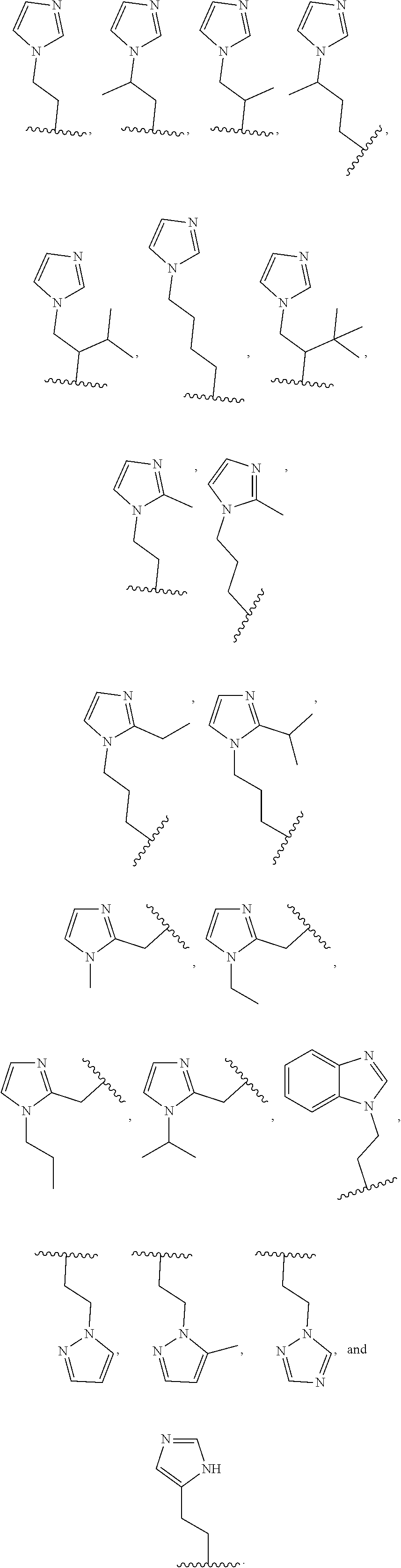

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl) (6-oxo-6-(undecyloxy) hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl) oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-IH-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within, or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; 1, 11, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-O13 or 503-O13 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 IZ)-heptatriaconta-6,9,28,3 I-tetraen-I9-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z, 16Z)-A,A-dimethyl-3-nonyldocosa-I3, I6-dien-I-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoyl phosphatidylcholine (DOPC), dipalmitoyl phosphatidylcholine (DPPC), dioleoyl phosphatidylglycerol (DOPG), dipalmitoyl phosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), I8-I-trans PE, I-stearoyl-2-oleoyl-phosphatidylethanolamine (SOPE), hydrogenated soy phosphatidylcholine (HSPC), egg phosphatidylcholine (EPC), dioleoyl phosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, Lys phosphatidylethanolamine, phosphatidylserine, phosphatidylinositol, sphingomyelin, egg sphingomyelin (ESM), cephalin, cardiolipin, phosphatidic acid, cerebrosides, dicetylphosphate, Lys phosphatidylcholine, dilinoleoylphosphatidylcholine, or mixtures thereof. It is understood that other diacyl phosphatidylcholine and diacyl phosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, non-phosphorous lipids such as, e.g., stearylamine, dodeeylamine, hexadecyl amine, acetyl palmitate, glycerol ricin oleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as I-(monomethoxy-polyethylene glycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-I-0-(w-methoxy(polyethoxy)ethyl) butanedioate (PEG-S-DMG)), PEG dialkoxypropylcarbam, N-(carbonyl-methoxypolyethylene glycol 2000)-I,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, US2018/0028664, and WO2017/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

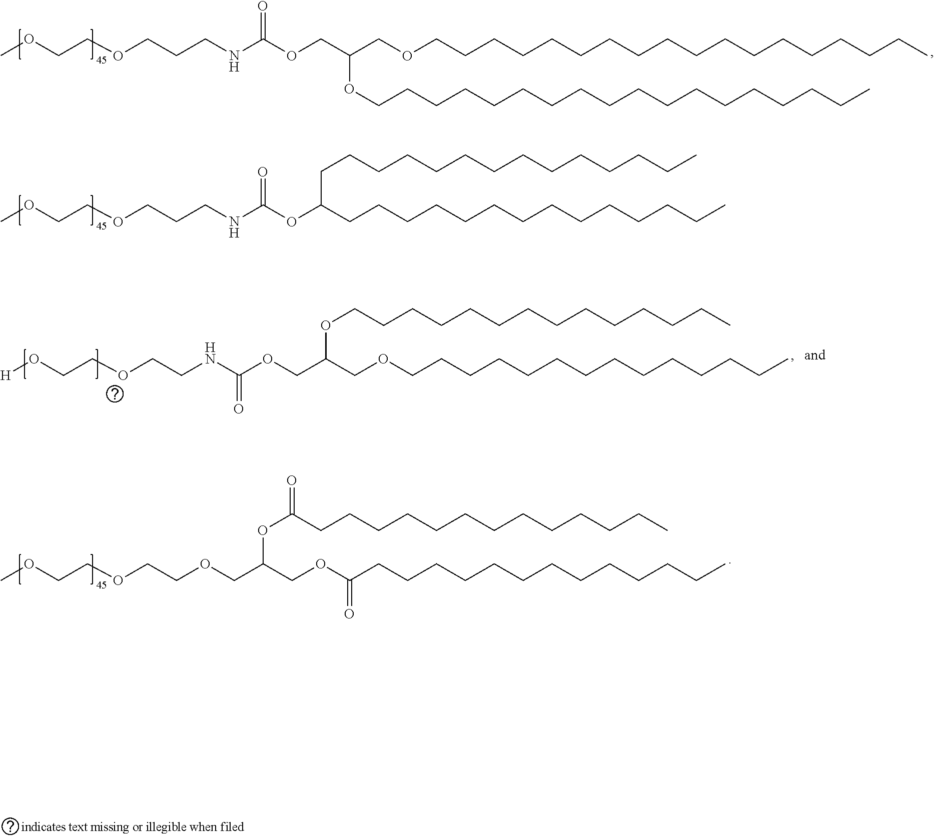

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates, and cationic polymer-lipids are described in the PCT, and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine, and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,I2Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,I2Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about I mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally include one or more coatings. In some embodiments, a LNP may be formulated in a capsule, film, or table having a coating. A capsule, film, or tablet including a composition described herein may have any useful size, tensile strength, hardness, or density.

Additional exemplary lipids, formulations, methods, and characterization of LNPs are taught by WO2020/061457 and WO2021/113777, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using LIPOFECTAMINE® MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew CHEM INT ED ENGL 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

In embodiments, a polyribonucleotide (e.g., a circular polyribonucleotide, a linear polyribonucleotide) encoding at least a portion (e.g., an antigenic portion) of a protein or polypeptide described herein is formulated in an LNP, wherein: (a) the LNPs include a cationic lipid, a neutral lipid, a cholesterol, and a PEG lipid, (b) the LNPs have a mean particle size of between 80 nm and 160 nm, and (c) the polyribonucleotide. In embodiments, the polyribonucleotide (e.g., circular polyribonucleotide, linear polyribonucleotide) formulated in an LNP is a vaccine.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). In some embodiments, a dose of a polyribonucleotide (e.g., a circular polyribonucleotide, a linear polyribonucleotide) antigenic composition described herein is between 30-200 mcg, e.g., 30 mcg, 50 mcg, 75 mcg, 100 mcg, 150 mcg, or 200 mcg.

### Kits

In some aspects, the disclosure provides a kit. In some embodiments, the kit includes (a) a circular polyribonucleotide, or a pharmaceutical composition described herein, and optionally (b) informational material. In some embodiments, the circular polyribonucleotide or pharmaceutical composition may be part of a defined dosing regimen. The informational material may be descriptive, instructional, marketing, or other material that relates to the methods described herein and/or the use of the pharmaceutical composition or circular polyribonucleotide for the methods described herein. The pharmaceutical composition or circular polyribonucleotide may comprise material for a single administration (e.g., single dosage form), or may comprise material for multiple administrations (e.g., a “multidose” kit).

The informational material of the kits is not limited in its form. In one embodiment, the informational material may include information about production of the pharmaceutical composition, the pharmaceutical drug substance, or the pharmaceutical drug product, molecular weight of the pharmaceutical composition, the pharmaceutical drug substance, or the pharmaceutical drug product, concentration, date of expiration, batch or production site information, and so forth. In one embodiment, the informational material relates to methods for administering a dosage form of the pharmaceutical composition. In one embodiment, the informational material relates to methods for administering a dosage form of the circular polyribonucleotide.

In addition to a dosage form of the pharmaceutical composition and circular polyribonucleotide described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a pharmaceutical composition or circular polyribonucleotide described herein. In such embodiments, the kit may include instructions for admixing a pharmaceutical composition or nucleic acid molecule (e.g., a circular polyribonucleotide) described herein and the other ingredients, or for using a pharmaceutical composition or nucleic acid molecule (e.g., a circular polyribonucleotide) described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a pharmaceutical composition or nucleic acid molecule (e.g., a circular polyribonucleotide) described herein may be provided in any form, e.g., liquid, dried or lyophilized form. It is preferred that a pharmaceutical composition or nucleic acid molecule (e.g., a circular polyribonucleotide) described herein be substantially pure and/or sterile. When a pharmaceutical composition or nucleic acid molecule (e.g., a circular polyribonucleotide) described herein is provided in a liquid solution, the liquid solution preferably is an aqueous solution, with a sterile aqueous solution being preferred. When a pharmaceutical composition or nucleic acid molecule (e.g., a circular polyribonucleotide) described herein is provided as a dried form, reconstitution generally is by the addition of a suitable solvent. The solvent, e.g., sterile water or buffer, can optionally be provided in the kit.

The kit may include one or more containers for the composition containing a dosage form described herein. In some embodiments, the kit contains separate containers, dividers or compartments for the composition and informational material. For example, the pharmaceutical composition or circular polyribonucleotide may be contained in a bottle, vial, or syringe, and the informational material may be contained in a plastic sleeve or packet. In other embodiments, the separate elements of the kit are contained within a single, undivided container. For example, the dosage form of a pharmaceutical composition or nucleic acid molecule (e.g., a circular polyribonucleotide) described herein is contained in a bottle, vial or syringe that has attached thereto the informational material in the form of a label. In some embodiments, the kit includes a plurality (e.g., a pack) of individual containers, each containing one or more unit dosage forms of a pharmaceutical composition or circular polyribonucleotide described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administered divided doses, as needed.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their invention.

### Example 1: Linear RNA Pulldown Methods for Enrichment of Circular RNA

This example describes the design of the method for removing linear RNA byproducts from circular RNA generated by self-splicing.

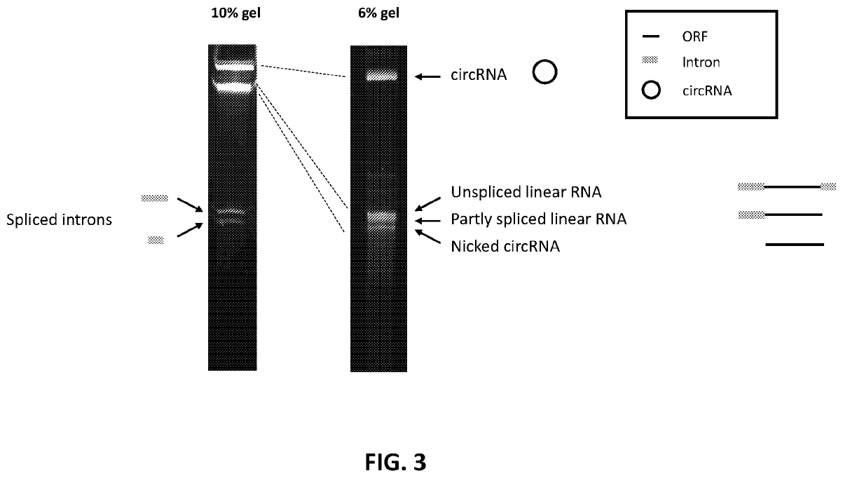

There may be several major linear byproducts in the in vitro transcription (IVT) mixture when circular RNA is generated by self-splicing: unspliced linear RNA, partly spliced linear RNA, fully spliced but unligated linear RNA, and spliced introns (FIG. 3).

In embodiments, a linear polyribonucleotide is designed to contain an aptamer near the 5′ end (FIG. 1). In other embodiments, a linear polyribonucleotide is designed to contain an aptamer at the 3′ end. The linear polyribonucleotide is circularized thereby producing a circular polyribonucleotide that does not include the aptamer. A reagent conjugated to a particle is added to the mixture. The reagent binds to the aptamer on the linear polyribonucleotides while the circular polyribonucleotides are not bound by the reagent, thereby separating the linear polyribonucleotides containing the aptamer from the circular polyribonucleotides that lack the aptamer.

In embodiments, a linear polyribonucleotide is designed to contain a circularization element (e.g., intron fragment) near the 5′ end (FIG. 2). A polyribonucleotide containing an aptamer also contains a region that hybridizes to the circularization element. The linear polyribonucleotide is circularized thereby producing a circular polyribonucleotide that does not include the circularization element that hybridizes to the aptamer. A reagent conjugated to a particle is added to the mixture. The reagent binds to the aptamer that is hybridized to linear polyribonucleotides while the circular polyribonucleotides are not bound by the reagent, thereby separating the linear polyribonucleotides containing the aptamer from the circular polyribonucleotides that lack the aptamer.

### Example 2: Lambda Peptide can Capture Linear RNA Byproducts Containing BoxB Aptamer and Enrich Circular RNA

This example describes enrichment of circular RNA by capturing linear byproducts via BoxB aptamer-lambda peptide interactions. In this example, linear RNA has a BoxB aptamer at the 5′ end of 3′ half-intron that is spliced out during self-splicing.

In this example, the construct is designed to have a 3′ half of a catalytic intron, an exon fragment 2 (E2), a polyribonucleotide cargo including an ORF, an exon fragment 1 (E1), and a 5′ half of a catalytic intron. The construct has also an extended sequence including 15 nucleotides of the BoxB aptamer (5′-GCCCUGAAGAAGGGC-3′ (SEQ ID NO: 148) or 5′-GCCCUGAAAAAGGGC-3′ (SEQ ID NO: 149)) at the 5′ end.

For linear pull down to remove linear byproducts that contain a BoxB aptamer, a lambda peptide that binds BoxB aptamer is designed. The peptide also has biotin that is linked by triethylene glycol (TEG).

Linear RNA is synthesized by in vitro transcription using T7 RNA polymerase from a DNA template in the presence of 7.5 mM of NTP. Template DNA is removed by treating with DNase for 20 minutes. Synthesized linear RNA is purified with an RNA clean up kit (New England Biolabs, T2050). Self-splicing occurs during transcription; no additional reaction is required.

Self-spliced RNA (200 pmol) is mixed with 400 pmol of biotinylated peptide in the presence of 1× binding buffer (150 mM NaCl, 15 mM sodium citrate, 0.5 mM EDTA) (final RNA concentration is 400 nM, and peptide concentration is 800 nM). As a negative control, RNA without BoxB aptamer is used. The RNA-peptide mixtures are incubated for 30 minutes at room temperature (RT), then mixed with 100 μL of Streptavidin-SEPHAROSE® beads (Sigma). The mixture is incubated at RT for 1 hour on a rotor mix and the unbound fraction is collected by spinning down the SEPHAROSE® beads by centrifugation. The beads are washed three times with 1× binding buffer. RNA bound to the beads is eluted by heating the beads for 10 minutes at 75° C. in the presence of 1× binding buffer. RNA still bound to the beads is eluted by heating the beads for 5 minutes at 95° C. in the presence of 95% formamide. The concentration of unbound and eluted RNA is measured by Qubit assay, and 200 ng of RNA is separated by urea polyacrylamide gel electrophoresis (Urea PAGE), stained by using gel stain and visualized using an imaging system. RNA bound to the beads is linear RNA that contains the BoxB aptamer, indicating that lambda peptide specifically captures the linear RNA with BoxB aptamer.

### Example 3. Tetracycline can Capture Linear RNA Byproducts Containing a Tetracycline Aptamer and Enrich Circular RNA

This example describes enrichment of circular RNA by capturing linear byproducts via tetracycline-tetracycline aptamer interactions. In this example, linear RNA has a tetracycline aptamer at the 5′ end of 3′ half-intron that is spliced out during self-splicing.

In this example, the construct is designed to have a 3′ half of a catalytic intron, an exon fragment 2 (E2), a polyribonucleotide cargo including an ORF, an exon fragment 1 (E1), and a 5′ half of a catalytic intron. The construct has an extended sequence including 60 nucleotides of the tetracycline aptamer at the 5′ end.

For linear pull down to remove linear byproducts that contain a tetracycline aptamer (5′-GGCCUAAAACAUACCAGAUUUCGAUCUGGAGAGGUGAAGAAUUCGACCACCUAGGCCGGU-3′ (SEQ ID NO: 150)), agarose beads conjugated with tetracycline are used.

Linear RNA is synthesized by in vitro transcription using T7 RNA polymerase from a DNA template in the presence of 7.5 mM of NTP. Template DNA is removed by treating DNase for 20 minutes. Synthesized linear RNA is purified with an RNA clean up kit (New England Biolabs, T2050). Self-splicing occurs during transcription; no additional reaction is required.

Self-spliced RNA (200 pmol) is mixed with 200 μL of tetracycline conjugated to agarose beads in the presence of 1× binding buffer (150 mM NaCl, 15 mM sodium citrate, 0.5 mM EDTA) (final RNA concentration is 400 nM). As a negative control, RNA without the tetracycline aptamer is used. The mixture is incubated at RT for 2 hours on a rotor mix and unbound fraction is collected by spinning down the agarose beads by centrifugation. The beads are washed three times with 1× binding buffer. RNA bound to the beads is eluted by heating the resin for 10 minutes at 75° C. in the presence of 1× binding buffer. RNA still bound to the beads is eluted by heating the beads for 5 minutes at 95° C. in the presence of 95% formamide. The concentration of unbound and eluted RNA is measured by Qubit assay, and 200 ng of RNA is separated by Urea PAGE, stained using gel stain, and visualized using an imaging system. RNA bound to the beads is linear RNA that contains the tetracycline aptamer, indicating that tetracycline specifically captures the linear RNA with the tetracycline aptamer.

### Example 4. Attaching BoxB Aptamer to the Linear RNA Byproducts to Enrich Circular RNA

This example describes enrichment of circular RNA by attaching a BoxB aptamer to linear RNA byproducts to capture linear RNA via BoxB aptamer-lambda peptide interaction. In this example, BoxB aptamer containing oligomer has a 23-nucleotide extended sequence that is complementary to the 5′ end of the 3′ half-intron sequence.

In this example, the construct is designed to have a 3′ half of a catalytic intron, an exon fragment 2 (E2), a polyribonucleotide cargo including an ORF, an exon fragment 1 (E1), and a 5′ half of a catalytic intron. The oligomer is designed to have a 15-nucleotide BoxB sequence (5′-GCCCUGAAGAAGGGC-3′ (SEQ ID NO: 151) or 5′-GCCCUGAAAAAGGGC-3′ (SEQ ID NO: 152)) and a 23-nucleotide extended sequence that is complementary to the 5′ end of a 3′ half-intron sequence.

For linear pull down to remove linear byproducts attached to the BoxB aptamer, a lambda peptide that binds BoxB aptamer is designed. The peptide has biotin that is linked by TEG.

Linear RNA is synthesized by in vitro transcription using T7 RNA polymerase from a DNA template in the presence of 7.5 mM of NTP. Template DNA is removed by treating DNase for 20 minutes. Synthesized linear RNA is purified with an RNA clean up kit (New England Biolabs, T2050). Self-splicing occurs during transcription; no additional reaction is required.

Self-spliced RNA (200 pmol) is mixed with 400 pmol oligomers harboring the BoxB aptamer and complementary sequence to the intron in the presence of 1× binding buffer (150 mM NaCl, 15 mM sodium citrate, 0.5 mM EDTA) (final RNA concentration is 400 nM, and oligomer concentration is 800 nM). The RNA-oligomer mixtures are incubated for 30 minutes at RT, then 800 pmol of biotinylated peptide is added to the RNA-oligomer mixture. As a negative control, oligomer without the BoxB aptamer is used. The RNA-peptide mixtures are incubated for 30 minutes at RT, then mixed with 100 μL of Streptavidin-SEPHAROSE® (Sigma). The mixture is incubated at RT for 1 hour on a rotor mix, and the unbound fraction is collected by spinning down SEPHAROSE® beads by centrifugation. The beads are washed three times with 1× binding buffer. RNA bound to the beads is eluted by heating the resin for 10 minutes at 75° C. in the presence of 1× binding buffer. RNA still bound to the beads is eluted by heating the beads for 5 minutes at 95° C. in the presence of 95% formamide. The concentration of unbound and eluted RNA is measured by Qubit assay, and 200 ng of RNA are separated by Urea PAGE, stained using gel stain, and visualized using an imaging system. RNA bound to the beads is linear RNA that attached to the BoxB aptamer, indicating that lambda peptide specifically captures the linear RNA attached to the BoxB aptamer.

### Example 5. Attaching Tetracycline Aptamer to the Linear RNA Byproducts to Enrich Circular RNA

This example describes enrichment of circular RNA by attaching a tetracycline aptamer to linear RNA byproducts to capture linear RNA via tetracycline aptamer-tetracycline interaction. In this example, a tetracycline aptamer containing oligomer has a 23-nucleotide extended sequence that is complementary to the 5′ end of a 3′ half-intron sequence.

In this example, the construct is designed to have a 3′ half of a catalytic intron, an exon fragment 2 (E2), a polyribonucleotide cargo including an ORF, an exon fragment 1 (E1), and a 5′ half of a catalytic intron. The oligomer is designed to have a 60-nucleotide tetracycline aptamer sequence (5′-GGCCUAAAACAUACCAGAUUUCGAUCUGGAGAGGUGAAGAAUUCGACCACCUAGGCCGGU-3′ (SEQ ID NO: 153)) and a 23-nucleotide extended sequence that is complementary to the 5′ end of the 3′ half-intron sequence.

For linear pull down to remove linear byproducts attached to the tetracycline aptamer, agarose beads conjugated with tetracycline are used.

Linear RNA is synthesized by in vitro transcription using T7 RNA polymerase from a DNA template in the presence of 7.5 mM of NTP. Template DNA is removed by treating DNase for 20 minutes. Synthesized linear RNA is purified with an RNA clean up kit (New England Biolabs, T2050). Self-splicing occurs during transcription; no additional reaction is required.

Self-spliced RNA (200 pmol) is mixed with 400 pmol of oligomers harboring tetracycline aptamer and a complementary sequence to the intron in the presence of 1× binding buffer (150 mM NaCl, 15 mM Sodium citrate, 0.5 mM EDTA) (final RNA concentration is 400 nM, and oligomer concentration is 800 nM). As a negative control, an RNA oligomer without tetracycline aptamer is used. The RNA-oligomer mixtures are incubated for 30 minutes at RT, then 200 μL of tetracycline conjugated agarose beads are added to the RNA-oligomer mixture and incubated at RT for 2 hours on a rotor mix. Unbound fraction is collected by spinning down the agarose beads by centrifugation. The beads are washed three times with 1× binding buffer. RNA bound to the beads is eluted by heating the resin for 10 minutes at 75° C. in the presence of 1× binding buffer. RNA still bound to the beads is eluted by heating the beads for 5 minutes at 95° C. in the presence of 95% formamide. The concentration of unbound and eluted RNA is measured by Qubit assay, and 200 ng of RNA are separated by Urea PAGE, stained using gel stain, and visualized using an imaging system. RNA bound to the beads is linear RNA that is attached to the tetracycline aptamer, indicating that tetracycline specifically captures the linear RNA attached to the tetracycline aptamer.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of separating a linear polyribonucleotide comprising an aptamer from a plurality of polyribonucleotides comprising a mixture of linear polyribonucleotides and circular polyribonucleotides, the method comprising:
(a) providing a sample comprising the plurality of polyribonucleotides, wherein a subset of the plurality of polyribonucleotides comprise the linear polyribonucleotide comprising the aptamer;
(b) contacting the sample with a reagent that binds to the aptamer; and
(c) separating the linear polyribonucleotide comprising the aptamer bound to the reagent from the plurality of polyribonucleotides.

2. The method of claim 1, wherein the linear polyribonucleotide comprising the aptamer is transcribed from a deoxyribonucleotide encoding the linear polyribonucleotide comprising the aptamer.

3. The method of claim 1, further comprising the step of producing the linear polyribonucleotide comprising the aptamer by attaching the aptamer to the linear polyribonucleotide.

4. A method of separating a linear polyribonucleotide from a plurality of polyribonucleotides comprising a mixture of linear polyribonucleotides and circular polyribonucleotides, the method comprising:
(a) providing a sample comprising the plurality of polyribonucleotides, wherein a subset of the plurality of polyribonucleotides comprise the linear polyribonucleotide;
(b) attaching an aptamer to the linear polyribonucleotide;
(c) contacting the sample with a reagent that binds to the aptamer; and
(d) separating the linear polyribonucleotide comprising the aptamer bound to the reagent from the plurality of polyribonucleotides.

5. The method of claim 3 or 4, wherein attaching the aptamer to the linear polyribonucleotide comprises covalently attaching the aptamer to a 3′ or 5′ terminus of the linear polyribonucleotide.

6. The method of claim 3 or 4, wherein attaching the aptamer to the linear polyribonucleotide comprises hybridizing the aptamer to a region of the linear polyribonucleotide.

7. The method of any one of claims 1-6, wherein the circular polyribonucleotides lack the aptamer.

8. The method of any one of claims 1-7, wherein the step of separating comprises collecting a portion of the sample that is not bound by the reagent.

9. The method of claim 7 or 8, wherein the portion of the sample that is not bound by the reagent comprises the circular polyribonucleotide.

10. The method of any one of claims 1-9, where the reagent is a polypeptide, a small molecule, a lipid, a carbohydrate, an RNA, or a metal.

11. The method of claim 10, where the reagent is a polypeptide.

12. The method of claim 11, wherein the polypeptide is selected from Table 1.

13. The method of claim 12, wherein the aptamer comprises a nucleic acid sequence selected from any one of SEQ ID NOs: 1-66.

14. The method of claim 11, wherein the polypeptide is Protein A, streptavidin, lambda peptide, or MS2 bacteriophage coat protein.

15. The method of claim 10, wherein the reagent is a small molecule.

16. The method of claim 15, wherein the small molecule is selected from Table 2.

17. The method of claim 16, wherein the aptamer comprises a nucleic acid sequence selected from any one of SEQ ID NOs: 67-119.

18. The method of claim 10, wherein the small molecule is biotin or tetracycline.

19. The method of claim 18, wherein the reagent is an RNA.

20. The method of claim 19, wherein the RNA is selected from Table 3.

21. The method of claim 20, wherein the aptamer comprises a nucleic acid sequence selected from SEQ ID NO: 120 or 121.

22. The method of claim 10, wherein the reagent is a metal.

23. The method of claim 22, wherein the metal is selected from Table 4.

24. The method of claim 23, wherein the aptamer comprises a nucleic acid sequence selected from any one of SEQ ID NOs: 122-124.

25. The method of any one of claims 1-24, wherein the step of separating comprises immobilizing the reagent.

26. The method of claim 25, wherein the reagent is conjugated to a particle.

27. The method of claim 26, wherein the particle comprises a magnetic bead.

28. The method of claim 26, wherein the reagent is conjugated to a resin comprising a plurality of the particles.

29. The method of claim 28, wherein the resin comprises cross-linked poly[styrene-divinylbenzene], agarose, or SEPHAROSE®.

30. The method of claim 28 or 29, wherein a column comprises the resin.

31. The method of claim 30, wherein the method comprises contacting the sample with the column and collecting an eluate comprising a portion of the sample that is not bound to the reagent from the plurality of polyribonucleotides in the sample.

32. The method of any one of claims 1-31, wherein the method further comprises, prior to step (a), providing a linear precursor polyribonucleotide and circularizing the linear precursor to produce the circular polyribonucleotide.

33. The method of claim 32, wherein the linear precursor comprises a 5′ self-splicing intron fragment and a 3′ self-splicing intron fragment, and wherein the circular polyribonucleotide is produced by self-splicing of the linear precursor.

34. The method of claim 33, wherein the 5′ self-splicing intron fragment and the 3′ self-splicing intron fragment are each a Group I or Group II self-splicing intron fragment.

35. The method of claim 32, wherein circularizing the circular polyribonucleotide is produced by splint-ligation of the linear precursor.

36. The method of any one of claims 1-35, wherein the circular polyribonucleotide comprises an ORF.

37. The method of claim 36, wherein the ORF encodes a polypeptide.

38. The method of claim 36 or 37, wherein a level of expression from the ORF of the circular polyribonucleotide after purification is increased at least 10% relative to a level of expression from the ORF prior to separating.

39. The method of any one of claims 36-38, wherein the circular polyribonucleotide comprises an internal ribosome entry site (IRES).

40. The method of claim 39, wherein the ORF is operably linked to the IRES.

41. The method of any one of claims 1-40, wherein the step of separating further comprises washing the polyribonucleotide comprising the aptamer that is bound to the reagent one or more times.

42. The method of any one of claims 1-41, wherein the step of separating further comprises eluting the polyribonucleotide comprising the aptamer from the reagent.

43. The method of any one of claims 1-42, wherein the method comprises providing a plurality of reagents, wherein each reagent binds to a distinct aptamer region.

44. The method of any one of claims 1-43, wherein the method comprises providing the reagent at a molar ratio of 10:1 to 1:10 to the polyribonucleotide comprising the aptamer region.

45. The method of any one of claims 1-44, wherein the method separates at least 500 μg of the circular polyribonucleotide.

46. The method of claim 45, wherein the method separates from 500 μg to 1000 mg of the circular polyribonucleotide.

47. A population of polyribonucleotides produced by the method of any one of claims 1-46.

48. The population of polyribonucleotides of claim 47, wherein the population comprises a circular polyribonucleotide lacking the aptamer and the circular polyribonucleotide comprises at least 40% (mol/mol) of the total polyribonucleotides in the composition.

49. The population of polyribonucleotides of claim 47 or 48, wherein the population comprises less than 40% (mol/mol) linear polyribonucleotides of the total polyribonucleotides in the composition.

50. The population of polyribonucleotides of claim 49, wherein the population comprises less than 30%, 20%, 10%, 5%, or 1% (mol/mol) linear polyribonucleotides of the total polyribonucleotides in the composition.

51. The population of polyribonucleotides of any one of claims 47-50, wherein a total weight of polyribonucleotides in the population of polyribonucleotides at least 500 μg.

52. The population of polyribonucleotides of claim 51, wherein the total weight of polyribonucleotides in the population of polyribonucleotides is from 500 μg to 1000 mg.

53. A pharmaceutical composition comprising the population of polyribonucleotides of any one of claims 47-52 and a diluent, carrier, or excipient.


SEQUENCE LISTING (153 sequences)

SEQ ID NO: 1 - RNA, 68 nt, synthetic construct
  source 1..68: mol_type = other RNA
gcaatggtacggtacttcctttggaagatagctggagaactaaccaaaagtgcacgctactttgctaa

SEQ ID NO: 2 - RNA, 66 nt, synthetic construct
  source 1..66: mol_type = other RNA
gggagagcggaagcgtgctgggccagtagtgtatagggctcgaaatgttcatggctcagtggacat

SEQ ID NO: 3 - RNA, 71 nt, synthetic construct
  source 1..71: mol_type = other RNA
gggaacagtccgagcctcactgttatccgatagcagcgcgggatgagggtcaatgcgtcataggatcccgc

SEQ ID NO: 4 - RNA, 73 nt, synthetic construct
  source 1..73: mol_type = other RNA
gggagaattccgaccagaagcttcgggatttgagggtagaatgggactacctttcctctctccttcctcttct

SEQ ID NO: 5 - RNA, 77 nt, synthetic construct
  source 1..77: mol_type = other RNA
gggagctcagaataaacgctcaaccagtcttgtggctttgaaagagaggagtgttcgacatgaggcccggatccggc

SEQ ID NO: 6 - RNA, 79 nt, synthetic construct
  source 1..79: mol_type = other RNA
gaattccgcgtgtgccagcgaaagttgcgtatgggtcacatcgcaggcacatgtcatctgggcggtccgttcgggatcc

SEQ ID NO: 7 - RNA, 90 nt, synthetic construct
  source 1..90: mol_type = other RNA
gggttcactgcagacttgacgaagcttccggagagaagggtcaaagttgtgcgggagtgtgttgtggaatggatccacatctacgaattc

SEQ ID NO: 8 - RNA, 107 nt, synthetic construct
  source 1..107: mol_type = other RNA
gataatacgactcactatagggttcactgcagacttgacgaagcttgcagaaaagggggaagaagagggtgattcaggcgagagaatggatccacatctacgaattc

SEQ ID NO: 9 - RNA, 121 nt, synthetic construct
  source 1..121: mol_type = other RNA
gggagaattccgaccagaagcttactgtcctcccttcagagagcgcgggacccttaacttggggcccacgaacagcttcagttccgtctcggcgtcatatgtgcgtctacatggatcctca

SEQ ID NO: 10 - RNA, 123 nt, synthetic construct
  source 1..123: mol_type = other RNA
gggagagccatacctgacaaagacgctagcgaattggttcctcactcaaaagtaggggaaagcgttattaagaaaccaaaatttgacaggttacgcatcctgcatcctccaggttacgcatcc

SEQ ID NO: 11 - RNA, 34 nt, synthetic construct
  source 1..34: mol_type = other RNA
ggtgaaggcaacgtataggcaagcacacttcacc

SEQ ID NO: 12 - RNA, 40 nt, synthetic construct
  source 1..40: mol_type = other RNA
ggagacatccctcttgttggtggtgccgtgtggatgtctc

SEQ ID NO: 13 - RNA, 79 nt, synthetic construct
  source 1..79: mol_type = other RNA
gggaatggatccacatctacgaattcatcagggctaaagaggcagagttacttagttcactgcagacttgacgaagctt

SEQ ID NO: 14 - RNA, 44 nt, synthetic construct
  source 1..44: mol_type = other RNA
gcctgtaaggtggtcggtgtggcgagtgtgttaggagagattgc

SEQ ID NO: 15 - RNA, 73 nt, synthetic construct
  source 1..73: mol_type = other RNA
gggcgctaagtcctcgctcatgcgcgtcccatggggtatagaggggtcgaagtggacgcgcgactcggatcct

SEQ ID NO: 16 - RNA, 92 nt, synthetic construct
  source 1..92: mol_type = other RNA
gggatgcttcggcatccccgaagccgctatggaccagtggcgcggcttcggcccgacggagtggtaccgcttcggcggtacgtaagcttggg

SEQ ID NO: 17 - RNA, 97 nt, synthetic construct
  source 1..97: mol_type = other RNA
gggatatcctcgagcataagaaacaagatagatcctgaaactgttttaaggttggccgatcttctgctcgagaatgcatgaagcgttccatattttt

SEQ ID NO: 18 - RNA, 99 nt, synthetic construct
  source 1..99: mol_type = other RNA
gtgagaccagccgagtggtgtctggctattcactggagcgtgggtggaacccctgcgcactcgtttggctgtccgggccttcgggccgggattatctct

SEQ ID NO: 19 - RNA, 112 nt, synthetic construct
  source 1..112: mol_type = other RNA
gggagataccagcttattcaattctggcaatgggctatcccaagtgctaggcttcagggagcgaggaccagacgacgtacctaaccctaaggtgagatagtaagtgcaatct

SEQ ID NO: 20 - RNA, 109 nt, synthetic construct
  source 1..109: mol_type = other RNA
gggagagccatacctgacaaagacgctagcgaattggttcctcactcaaaagtaggggaaagcgttattaagaaaccaaaatttgacaggttacgcatcctgcatcctc

SEQ ID NO: 21 - RNA, 33 nt, synthetic construct
  source 1..33: mol_type = other RNA
ttaccacgcgctcttaactgctagcgccatggc

SEQ ID NO: 22 - RNA, 37 nt, synthetic construct
  source 1..37: mol_type = other RNA
ttcaacgctgtgaagggcttatacgagcggattaccc

SEQ ID NO: 23 - RNA, 41 nt, synthetic construct
  source 1..41: mol_type = other RNA
gggagtctgaagtcggacttgttatcaattcacttcagact

SEQ ID NO: 24 - RNA, 91 nt, synthetic construct
  source 1..91: mol_type = other RNA
gggaggatattttctcagaccgtaagtaccgaatgtgcttttggccgatttttggcccctgcagttgcagcatcgtgaactaggatccggg

SEQ ID NO: 25 - RNA, 43 nt, synthetic construct
  source 1..43: mol_type = other RNA
cgtcccgtgcgcaaaagttcttagcgctagcagtcctagttgc

SEQ ID NO: 26 - RNA, 61 nt, synthetic construct
  source 1..61: mol_type = other RNA
tgttcatgtcctactgttcaaacaaaaaaactgtgcacaaaaataaattggggcatggaca

SEQ ID NO: 27 - RNA, 64 nt, synthetic construct
  source 1..64: mol_type = other RNA
ggtagatacgatggataccccctgtggcccgtcaacacaggggaagtggcatgacgcgcagcca

SEQ ID NO: 28 - RNA, 90 nt, synthetic construct
  source 1..90: mol_type = other RNA
gggttcactgcagacttgacgaagcttacaaacaagagcaaaaagggagttgacgtagactgtgcggaatggatccacatctacgaattc

SEQ ID NO: 29 - RNA, 28 nt, synthetic construct
  source 1..28: mol_type = other RNA
ggcagaccatacgggagagaaacttgcc

SEQ ID NO: 30 - RNA, 35 nt, synthetic construct
  source 1..35: mol_type = other RNA
ttgcttggaatgcgttatagtctcttaggtgtgta

SEQ ID NO: 31 - RNA, 37 nt, synthetic construct
  source 1..37: mol_type = other RNA
acgaagcttgatcccgtttgccggtcgatcgcttcga

SEQ ID NO: 32 - RNA, 42 nt, synthetic construct
  source 1..42: mol_type = other RNA
cctccggctcataccttttcgaagacaagcttcgacaggagg

SEQ ID NO: 33 - RNA, 80 nt, synthetic construct
  source 1..80: mol_type = other RNA
gggctcatgcacgtttgctcctgtaattggcgtatgtaacccaggcaccaaacaccccaggccgggccatgatccacata

SEQ ID NO: 34 - RNA, 75 nt, synthetic construct
  source 1..75: mol_type = other RNA
ggcgtgacctgatgagtcacgctaaggaggatttccgaaagcggctacggtccgccagtgttacgaaacgttccc

SEQ ID NO: 35 - RNA, 95 nt, synthetic construct
  source 1..95: mol_type = other RNA
ggcgtgacctgatgagtcacgcagacgctagcgaattggttcctcgaaaggggaaagcgttattaagaaaccaaaatgtgttacgaaacgttccc

SEQ ID NO: 36 - RNA, 95 nt, synthetic construct
  source 1..95: mol_type = other RNA
ggacctcggcgaaagctaacgtctcatggctaaattgccatgttgctacaaatgatatgactagagaggttaggtgcctcgtgatgtccagtcgc

SEQ ID NO: 37 - RNA, 88 nt, synthetic construct
  source 1..88: mol_type = other RNA
gggagacaagaataaacgctcaacacagaacgcggtccccacacaggacaggagccagccccggttcgacaggaggctcacaacaggc

SEQ ID NO: 38 - RNA, 44 nt, synthetic construct; identical to SEQ ID NO: 14
  source 1..44: mol_type = other RNA

SEQ ID NO: 39 - RNA, 58 nt, synthetic construct
  source 1..58: mol_type = other RNA
gggcgaattcaactgccatctaggcagtaaccaggagttagtaggacaagtttcgtcc

SEQ ID NO: 40 - RNA, 79 nt, synthetic construct
  source 1..79: mol_type = other RNA
gggcgaattccgcgtgtgctgagggcggaagaactaatttgggacggattgcggccgttgtctgtggcgtccgttcggg

SEQ ID NO: 41 - RNA, 80 nt, synthetic construct
  source 1..80: mol_type = other RNA
gaagctttcacacaacaaggcccgggactgtattagggaaattagagtacagacagtcgccgtgggtcgaattccgctca

SEQ ID NO: 42 - RNA, 83 nt, synthetic construct
  source 1..83: mol_type = other RNA
gaagcttacaagaaggacagcacgaataaaacctgcgtaaatccgccccatttgtgtaagggtagtgggtcgaattccgctca

SEQ ID NO: 43 - RNA, 85 nt, synthetic construct
  source 1..85: mol_type = other RNA
gcatgcagtgtctattctcgagtagcgatcgttgaaggggtataaggttggcagatcgctagcatgcaactgactcggataagca

SEQ ID NO: 44 - RNA, 114 nt, synthetic construct
  source 1..114: mol_type = other RNA
gggagctcagccttcactgcactccggctggtggacgcggtacgagcaatttgtaccggatggatgttcgggcagcggtgtggcagggatgagcggcaccacggtcggatccac

SEQ ID NO: 45 - RNA, 104 nt, synthetic construct
  source 1..104: mol_type = other RNA
gggcgaattcgggacttcgatccgtagtacccaccaggcatacaccagcacgcggagccaaggaaaaatagtaaactagcactcagtgctcgtatgcggaagct

SEQ ID NO: 46 - RNA, 63 nt, synthetic construct
  source 1..63: mol_type = other RNA
ggaaagacgctagcgaattggttcctcgaaaggggaaagcgttattaagaaaccaaaatttcc

SEQ ID NO: 47 - RNA, 81 nt, synthetic construct
  source 1..81: mol_type = other RNA
ggacgcgtggtaccaggccgatctatggacgctataggcacaccggatactttaacgattggctaagcttccgcggggatc

SEQ ID NO: 48 - RNA, 106 nt, synthetic construct
  source 1..106: mol_type = other RNA
ggaagaaagaggtctgagacattctcttataggggaggctgtggtgagggaatattaagagaattaacggtctagttcacctcgacttctggagttgacgttgctt

SEQ ID NO: 49 - RNA, 42 nt, synthetic construct
  source 1..42: mol_type = other RNA
tagtgtgagagccgtgagtgaaaggccgcgacaaagatcgga

SEQ ID NO: 50 - RNA, 37 nt, synthetic construct
  source 1..37: mol_type = other RNA
gggttttcgggatacgcggagggtgggcaataacccc

SEQ ID NO: 51 - RNA, 51 nt, synthetic construct
  source 1..51: mol_type = other RNA
gggaggacgatgcgggccgttgttgtgtgccgccccagacgactcgcccga

SEQ ID NO: 52 - RNA, 51 nt, synthetic construct
  source 1..51: mol_type = other RNA
gggaggacgatgcggtcctgtcgtctgttcgtccccagacgactcgcccga

SEQ ID NO: 53 - RNA, 51 nt, synthetic construct
  source 1..51: mol_type = other RNA
gggaggacgatgcggtgagtcgttcccttcgtccccagacgactcgcccga

SEQ ID NO: 54 - RNA, 76 nt, synthetic construct
  source 1..76: mol_type = other RNA
ataccagcttattcaattgcctgaaaagctatcgcccaattcgcagtgatatcctttaagatagtaagtgcaatct

SEQ ID NO: 55 - RNA, 84 nt, synthetic construct
  source 1..84: mol_type = other RNA
cccatccctcttcctctctccctcggtactgctacagcaatgcatctacgtctctgtggattcgacgactcgctgagatcgaga

SEQ ID NO: 56 - RNA, 85 nt, synthetic construct
  source 1..85: mol_type = other RNA
gggagagcggaagcgtgctgggcctgtcgttcgctgtgtcttgctacgttacgttacacggttggcataacccagaggtcgatgg

SEQ ID NO: 57 - RNA, 99 nt, synthetic construct
  source 1..99: mol_type = other RNA
ggagctcagccttcactgcgatcaccgtgtcggtggctagtcgatgtatcccgatcaccattgggggcaagtttagacaggcaccacggtcggatccac

SEQ ID NO: 58 - RNA, 15 nt, synthetic construct
  source 1..15: mol_type = other RNA
ccctcctacataggg

SEQ ID NO: 59 - RNA, 19 nt, synthetic construct
  source 1..19: mol_type = other RNA
cagaacgtatactattctg

SEQ ID NO: 60 - RNA, 74 nt, synthetic construct
  source 1..74: mol_type = other RNA
gggagaattccgaccagaagggttagcggtcgtcttaagtagtttttggtcctttcctctctccttcctcttct

SEQ ID NO: 61 - RNA, 75 nt, synthetic construct
  source 1..75: mol_type = other RNA
gggagaattccgaccagaagggttagcggtcgtcttaaggtagtttttggtcctttcctctctccttcctcttct

SEQ ID NO: 62 - RNA, 85 nt, synthetic construct
  source 1..85: mol_type = other RNA
ggaccgagaagttaccctgtaatcttaggatgaatcgcatgctctagcgaccttttcggcttcggcgtacgcacatcgcagcaac

SEQ ID NO: 63 - RNA, 15 nt, synthetic construct
  source 1..15: mol_type = other RNA
gccctgaagaagggc

SEQ ID NO: 64 - RNA, 15 nt, synthetic construct
  source 1..15: mol_type = other RNA
gccctgaaaaagggc

SEQ ID NO: 65 - RNA, 23 nt, synthetic construct
  source 1..23: mol_type = other RNA
aaacatgaggataacccatgtcg

SEQ ID NO: 66 - RNA, 23 nt, synthetic construct
  source 1..23: mol_type = other RNA
aaacatgaggatcacccatgtcg

SEQ ID NO: 67 - RNA, 90 nt, synthetic construct
  source 1..90: mol_type = other RNA
ggataatagccgtaggttgcgaaagcgaccctgatgagccctgcgatgcagaaaggtgctgacgacacatcgaaacggtagcgagagctc

SEQ ID NO: 68 - RNA, 104 nt, synthetic construct
  source 1..104: mol_type = other RNA
gggatcctaagctctatcggctggacgacggggacgccactggactaggtaagccaggaccgtacgtcgggagccgtcagaataaaagcggcctagcgatcgat

SEQ ID NO: 69 - RNA, 99 nt, synthetic construct
  source 1..99: mol_type = other RNA
ggatgtccagtcgcttgcaatgcccttttagaccctgatgaggatcatcggactttgtcctgtggagtaagatcgcgaaacggtgaaagccgtaggtct

SEQ ID NO: 70 - RNA, 99 nt, synthetic construct
  source 1..99: mol_type = other RNA
ggatgtccagtcgcttgcaatgcccttttagaccctgatgagcggtgctagttagttgcagtttcggttgttacgcgaaacggtgaaagccgtaggtct

SEQ ID NO: 71 - RNA, 117 nt, synthetic construct
  source 1..117: mol_type = other RNA
ggataatagccgtaggttgcgaaagcgaccctgatgagccttaggatatgtctggataccatgcatgatgcaccttggcagtcttacagaaggacgtcgaaacggtagcgagagctc

SEQ ID NO: 72 - RNA, 80 nt, synthetic construct
  source 1..80: mol_type = other RNA
gggaatggatccacatctacgaattcgaattgccgtaatttcccgtggagcgatgcttcactgcagacttgacgaagctt

SEQ ID NO: 73 - RNA, 38 nt, synthetic construct
  source 1..38: mol_type = other RNA
ggatcccgactggcgagagccaggtaacgaatggatcc

SEQ ID NO: 74 - RNA, 40 nt, synthetic construct
  source 1..40: mol_type = other RNA
gggttgggaagaaactgtggcacttcggtgccagcaaccc

SEQ ID NO: 75 - RNA, 87 nt, synthetic construct
  source 1..87: mol_type = other RNA
gggagacaagaataaacgctcaactgcgatcaggggtaaatttccgcgcaggctccacgccgcttcgacaggaggctcacaacaggc

SEQ ID NO: 76 - RNA, 91 nt, synthetic construct
  source 1..91: mol_type = other RNA
gggagaggatactacacgtgataggacgattatcgaaaatcaccagattggaccctggttaacgatccattgcatgtagcagaagcttccg

SEQ ID NO: 77 - RNA, 97 nt, synthetic construct
  source 1..97: mol_type = other RNA
ggagctcagccttcactgcatgataaaccgatgctgggcgattctcctgaagtaggggaagagttgtcatgtatgggggcaccacggtcggatcctg

SEQ ID NO: 78 - RNA, 93 nt, synthetic construct
  source 1..93: mol_type = other RNA
gggaattccgcgtgtgcgccgcggaagacgttggaaggatagatacctacaacggggaatatagaggccagcacatagtgaggccctcctccc

SEQ ID NO: 79 - RNA, 104 nt, synthetic construct
  source 1..104: mol_type = other RNA
ggaatggatccacatctacgaaggctttgaaggtgagaccgtgcaaatgaggatggtgtggatgattagggttgtcggttttcactgcagacttgacgaagctt

SEQ ID NO: 80 - RNA, 109 nt, synthetic construct
  source 1..109: mol_type = other RNA
gggagaattccgaccagaagcttagtatagcgaggtttagctacactcgtgctgatcgtttggtacgggacctgcgtgtagcccatatgtgcgtctacatggatcctca

SEQ ID NO: 81 - RNA, 32 nt, synthetic construct
  source 1..32: mol_type = other RNA
ggtcttacgtcgttcgcgactattgggagacc

SEQ ID NO: 82 - RNA, 31 nt, synthetic construct
  source 1..31: mol_type = other RNA
ggaagagatggcgactaaaacgacttgtcgc

SEQ ID NO: 83 - RNA, 32 nt, synthetic construct
  source 1..32: mol_type = other RNA
ggcacgtgtattaccctagtggtcgacgtgcc

SEQ ID NO: 84 - RNA, 35 nt, synthetic construct
  source 1..35: mol_type = other RNA
ccggtgcgcataaccacctcagtgcgagcaaggaa

SEQ ID NO: 85 - RNA, 40 nt, synthetic construct
  source 1..40: mol_type = other RNA
ggcatcggaaagtgggttgatgtaagtaacaggcgatgcc

SEQ ID NO: 86 - RNA, 42 nt, synthetic construct
  source 1..42: mol_type = other RNA
gggcggatgagacgcttggcgtgtgctgtggagagtcatccg

SEQ ID NO: 87 - RNA, 44 nt, synthetic construct
  source 1..44: mol_type = other RNA
gacgagaaggagtgctggttatactagcggttaggtcactcgtc

SEQ ID NO: 88 - RNA, 57 nt, synthetic construct
  source 1..57: mol_type = other RNA
gggagatctacggatctcagggctcttacgggagctacatggaaggagtccatgtgt

SEQ ID NO: 89 - RNA, 153 nt, synthetic construct
  source 1..153: mol_type = other RNA
agtaatacgactcactatagggagaattccgaccagaagttggcgttggcatgacgcggggaatcgggtgcatcgatgactactcctgggcccacgtctgttgttgacgtcacagcttgatttaggatagcgcttgggcagtcgtgcagtgga

SEQ ID NO: 90 - RNA, 38 nt, synthetic construct
  source 1..38: mol_type = other RNA
agtgataccagcatcgtcttgatgcccttggcagcact

SEQ ID NO: 91 - RNA, 49 nt, synthetic construct
  source 1..49: mol_type = other RNA
ggaacccaactaggcgtttgaggggattcggccacggtaacaacccctc

SEQ ID NO: 92 - RNA, 50 nt, synthetic construct
  source 1..50: mol_type = other RNA
gggatcacagtgaaaaaagacgtgtgaatgtcacactgaaaaaagatccc

SEQ ID NO: 93 - RNA, 60 nt, synthetic construct
  source 1..60: mol_type = other RNA
ggcctaaaacataccagatttcgatctggagaggtgaagaattcgaccacctaggccggt

SEQ ID NO: 94 - RNA, 80 nt, synthetic construct
  source 1..80: mol_type = other RNA
gggaatggatccacatctacgaattcaccgcggggttgcggaccgggagctccagcttcactgcagacttgacgaagctt

SEQ ID NO: 95 - RNA, 83 nt, synthetic construct
  source 1..83: mol_type = other RNA
gggagctcagaataaacgctcaaatccgtggacagggcgtaagcgccttcgacatgagacacggatcctgcgacgaattcagc

SEQ ID NO: 96 - RNA, 110 nt, synthetic construct
  source 1..110: mol_type = other RNA
ggaacactatccgatggcaccgaccataggctcgggttgccagaggttccacactttcatcgaaaagcctatgctaggcaatgacatggactccttggtcattaggatcg

SEQ ID NO: 97 - RNA, 111 nt, synthetic construct
  source 1..111: mol_type = other RNA
gggagctcggtaccgaattctcgccctataggggtgttgagggaaatgtgtgcgacaaggtgcggtggccagaacttttcgttctcatcaaaagctttgcagaggatcctt

SEQ ID NO: 98 - RNA, 44 nt, synthetic construct
  source 1..44: mol_type = other RNA
gacgagaaggagcgctggttctactagcaggtaggtcactcgtc

SEQ ID NO: 99 - RNA, 44 nt, synthetic construct
  source 1..44: mol_type = other RNA
ggtggagggggacgtgctgcatccgcagtgcgtcttgggttgtg

SEQ ID NO: 100 - RNA, 79 nt, synthetic construct
  source 1..79: mol_type = other RNA
gggtaggccaggcagccaactagcgagagcttaaatctctgagcccgagagggttcagtgctgcttatgtggacggctt

SEQ ID NO: 101 - RNA, 80 nt, synthetic construct
  source 1..80: mol_type = other RNA
attggatcggtagtatttagggtgagacacttcatgcctttgttgcaggctggggtgaaggcgctacatggcgtctgaaa

SEQ ID NO: 102 - RNA, 88 nt, synthetic construct
  source 1..88: mol_type = other RNA
ggataatagccgtaggttgcgaaagcgaccctgatgagccttaggatatgcatgatgcagaaggacgtcgaaacggtagcgagagctc

SEQ ID NO: 103 - RNA, 90 nt, synthetic construct
  source 1..90: mol_type = other RNA
ggataatagccgtaggttgcgaaagcgaccctgatgagcctttagggccaagtgtggtgaaagacacactcgaaacggtagcgagagctc

SEQ ID NO: 104 - RNA, 90 nt, synthetic construct
  source 1..90: mol_type = other RNA
ggataatagccgtaggttgcgaaagcgaccctgatgagcctgtggaaacagacgtggcacatgactacgtcgaaacggtagcgagagctc

SEQ ID NO: 105 - RNA, 94 nt, synthetic construct
  source 1..94: mol_type = other RNA
ggataatagccgtaggttgcgaaagcgaccctgatgagtctggataccatgcatgatgcaccttggcagtcttacgaaacggtagcgagagctc

SEQ ID NO: 106 - RNA, 49 nt, synthetic construct; identical to SEQ ID NO: 91
  source 1..49: mol_type = other RNA

SEQ ID NO: 107 - RNA, 72 nt, synthetic construct
  source 1..72: mol_type = other RNA
gggcgaccctgatgagccttaggatatgcttcggcagaaggacgtcgaaacggtgaaagccgtaggttgccc

SEQ ID NO: 108 - RNA, 76 nt, synthetic construct
  source 1..76: mol_type = other RNA
gggcgaccctgatgagccttataccagccgaaaggcccttggcagacgtcgaaacggtgaaagccgtaggttgccc

SEQ ID NO: 109 - RNA, 81 nt, synthetic construct
  source 1..81: mol_type = other RNA
gggcgaccctgatgagccttgggaagaaactgtggcacttcggtgccagcacgtcgaaacggtgaaagccgtaggttgccc

SEQ ID NO: 110 - RNA, 85 nt, synthetic construct
  source 1..85: mol_type = other RNA
gggcgaccctgatgagcacacgagggggaaaccccggacaatcagacacggtgttcgaaacggtgaaagccgtaggttgcccttt

SEQ ID NO: 111 - RNA, 45 nt, synthetic construct
  source 1..45: mol_type = other RNA
ggcacgtacgaaggaaggtttggtatgtggtatattcgtacgtgc

SEQ ID NO: 112 - RNA, 58 nt, synthetic construct
  source 1..58: mol_type = other RNA
gtctctgtgtgcgccagagaacactggggcagatatgggccagcacagaatgaggccc

SEQ ID NO: 113 - RNA, 71 nt, synthetic construct
  source 1..71: mol_type = other RNA
gggcgaccctgatgagatgaggatatgcttcggcagaaggctctcgaaacggtgaaagccgtaggttgccc

SEQ ID NO: 114 - RNA, 67 nt, synthetic construct
  source 1..67: mol_type = other RNA
gggaattccgcgtgtgcgccgcggaagagggaatatagaggccagcacatagtgaggccctcctccc

SEQ ID NO: 115 - RNA, 75 nt, synthetic construct
  source 1..75: mol_type = other RNA
gggcgaccctgatgagatgataccagccgaaaggcccttggcagctctcgaaacggtgaaagccgtaggttgccc

SEQ ID NO: 116 - RNA, 78 nt, synthetic construct
  source 1..78: mol_type = other RNA
gggcgaattcccgaggaccaaatagtaccacccgggaaaacagctaatgccgaaacggagattttttctgcagaagct

SEQ ID NO: 117 - RNA, 80 nt, synthetic construct
  source 1..80: mol_type = other RNA
gggcgaccctgatgagatggggaagaaactgtggcacttcggtgccagcctctcgaaacggtgaaagccgtaggttgccc

SEQ ID NO: 118 - RNA, 60 nt, synthetic construct
  source 1..60: mol_type = other RNA
gggctattgctggaggggcgccacatgaaagtggtggttgggtgcggtcggcgatagctc

SEQ ID NO: 119 - RNA, 60 nt, synthetic construct; identical to SEQ ID NO: 93
  source 1..60: mol_type = other RNA

SEQ ID NO: 120 - RNA, 109 nt, synthetic construct
  source 1..109: mol_type = other RNA
gggaattccgcgtgtgctacgtatcttcaggcggtaactaactgtgctgagtctaatctttgtgagggacggtaacatatggttcccgcgtggtccgttcgggatcctc

SEQ ID NO: 121 - RNA, 37 nt, synthetic construct
  source 1..37: mol_type = other RNA
ggtgataccgcccaagagttcatatcgacggcggtgt

SEQ ID NO: 122 - RNA, 89 nt, synthetic construct
  source 1..89: mol_type = other RNA
gggagaggatactacacgtggaaaaaccaacaaattgggaaaaatgttaagggtccacttcatgccattgcatgtagcagaagcttccg

SEQ ID NO: 123 - RNA, 98 nt, synthetic construct
  source 1..98: mol_type = other RNA
ggataatagccgtaggttgcgaaagcgaccctgatgagaaagccaaagccgtagcgcagatgatctcgccatcagtaccgaaacggtagcgagagctc

SEQ ID NO: 124 - RNA, 95 nt, synthetic construct
  source 1..95: mol_type = other RNA
gggagaggatactactgtcatacgttaggctgtaggcgaggtgaaatgagcggtaatagcctcagcgtagcatatgcatgaattcgaagcttcgc

SEQ ID NO: 125 - RNA, 10 nt, synthetic construct
  source 1..10: mol_type = other RNA
ccraccatgg

SEQ ID NO: 126 - protein, 6 aa, synthetic construct
CHYSEL

SEQ ID NO: 127 - protein, 9 aa, synthetic construct
  VARIANT 1: note = absent or G or H
  VARIANT 2: note = absent or D or G
  VARIANT 3: note = D or V or I or S or M
  VARIANT 5: note = any amino acid
XXXEXNPGP

SEQ ID NO: 128 - protein, 8 aa, synthetic construct
  VARIANT 2: note = or I
  VARIANT 4: note = any amino acid
DVEXNPGP

SEQ ID NO: 129 - protein, 9 aa, synthetic construct
GDVESNPGP

SEQ ID NO: 130 - protein, 9 aa, synthetic construct
GDIEENPGP

SEQ ID NO: 131 - protein, 7 aa, synthetic construct
VEPNPGP

SEQ ID NO: 132 - protein, 7 aa, synthetic construct
IETNPGP

SEQ ID NO: 133 - protein, 9 aa, synthetic construct
GDIESNPGP

SEQ ID NO: 134 - protein, 9 aa, synthetic construct
GDVELNPGP

SEQ ID NO: 135 - protein, 9 aa, synthetic construct
GDIETNPGP

SEQ ID NO: 136 - protein, 8 aa, synthetic construct
GDVENPGP

SEQ ID NO: 137 - protein, 9 aa, synthetic construct
GDVEENPGP

SEQ ID NO: 138 - protein, 9 aa, synthetic construct
GDVEQNPGP

SEQ ID NO: 139 - protein, 7 aa, synthetic construct
IESNPGP

SEQ ID NO: 140 - protein, 9 aa, synthetic construct
GDIELNPGP

SEQ ID NO: 141 - protein, 9 aa, synthetic construct
HDIETNPGP

SEQ ID NO: 142 - protein, 9 aa, synthetic construct
HDVETNPGP

SEQ ID NO: 143 - protein, 9 aa, synthetic construct
HDVEMNPGP

SEQ ID NO: 144 - protein, 9 aa, synthetic construct
GDMESNPGP

SEQ ID NO: 145 - protein, 9 aa, synthetic construct
GDVETNPGP

SEQ ID NO: 146 - protein, 9 aa, synthetic construct
GDIEQNPGP

SEQ ID NO: 147 - protein, 8 aa, synthetic construct
DSEFNPGP

SEQ ID NO: 148 - RNA, 15 nt, synthetic construct
  source 1..15: mol_type = other RNA
gccctgaagaagggc

SEQ ID NO: 149 - RNA, 15 nt, synthetic construct
  source 1..15: mol_type = other RNA
gccctgaaaaagggc

SEQ ID NO: 150 - RNA, 60 nt, synthetic construct; identical to SEQ ID NO: 93
  source 1..60: mol_type = other RNA

SEQ ID NO: 151 - RNA, 15 nt, synthetic construct
  source 1..15: mol_type = other RNA
gccctgaagaagggc

SEQ ID NO: 152 - RNA, 15 nt, synthetic construct
  source 1..15: mol_type = other RNA
gccctgaaaaagggc

SEQ ID NO: 153 - RNA, 60 nt, synthetic construct; identical to SEQ ID NO: 93
  source 1..60: mol_type = other RNA